(19) United States
(12) Patent Application Publication
(10) Pub. No.: US 2019/0217315 A1
(43) Pub. Date: Jul. 18, 2019

(54) ADAPTIVE ELECTROSPRAY DEVICE

(71) Applicant: Avectas Limited, Co. Kildare (IE)

(72) Inventors: Michael Maguire, Dublin (IE); Shane Finnegan, Dublin (IE)

(21) Appl. No.: 16/329,407

(22) PCT Filed: Aug. 31, 2017
(86) PCT No.: PCT/IB17/01236

§ 371 Date: Feb. 28, 2019

Related U.S. Application Data
(60) Provisional application No. 62/381,667, filed on Aug. 31, 2016.

(51) Int. Cl. B05B 5/00; B05B 5/025; B05B 12/08; B05B 12/18; C12N 11/08; A61K 9/50

(52) U.S. Cl. CPC B05B 5/006; B05B 5/0255; A61K 9/5089; B05B 12/18; C12N 11/08; B05B 12/082

(57) ABSTRACT

The current subject matter includes an adaptive electrospray device that creates consistent output when operating in atmosphere (e.g., not a vacuum). For example, the current subject matter includes an adaptive system that can monitor two current reference points (at the emitter and counter-electrode, respectively), determine a change in emitter current that will account for the parasitic losses, and adjust the emitter current accordingly. In addition, the current subject matter includes a high-throughput adaptive electrospray device having an array of emitters that rapidly switches the electrical potential of different emitters in an array on and off at a predetermined sequence that mitigates or eliminates interference from neighboring emitters. Related apparatus, systems, techniques and articles are also described.

### CROSS-REFERENCE TO RELATED APPLICATIONS

This application claims priority under 35 U.S.C. § 119 to U.S. Provisional Patent Application No. 62/381,667 filed Aug. 31, 2016, the entire contents of which is hereby expressly incorporated by reference herein.

### TECHNICAL HELD

The subject matter described herein relates to adaptive electrospray technology that provides reliable output.

### BACKGROUND

An electrospray system is a system that utilizes electrical potential (voltage) to disperse a liquid across a gap between a conductive source emitter and a conductive counter electrode. Electrospray systems are generally implemented with a single emitter. The character of the electrospray output by such a system is generally characterized by the electrospray mode. Within, for example, cone jet mode, the electrospray is characterized in terms of a number of parameters, including electrospray particle droplet or colloid size, particle time of flight between creation and deposition on a counter electrode, evaporation rate of the particle, monodispersity of particles, dispersion pattern, charge retained on the surface of particles, electrospray current flowing between the emitter and the counter electrode carried by particles, the oscillations of the Taylor cone issuing droplets with reference to voltage, current and charge frequency components, and chemical ion transport within the solution being electrosprayed. Electrospraying of chemical solutions is a complex phenomenon with a number of interdependent parameters. As a result, the outputs of electrospray systems are inconsistent and can vary significantly, for example, due to day-to-day humidity variation.

### SUMMARY

The current subject matter includes an adaptive electrospray device that creates consistent output when operating in atmosphere (e.g., not a vacuum). Because electrospray output depends on current flowing from an emitter to a counter-electrode (also referred to as a collector), ambient humidity causes parasitic current losses. The ionization current is not routinely used as a characterization parameter. The current subject matter includes devices that use ionization current as a characterization parameter of operation. For example, the current subject matter includes an adaptive system that can monitor two current reference points (at the emitter and counter-electrode, respectively), determine a change in emitter current that will account for the parasitic losses, and adjust the emitter current accordingly.

In addition, the current subject matter includes adaptive electrospray devices having an array of emitters. Where a single emitter/counter-electrode pair are used, the electric potential difference between the emitter and counter-electrode create an electric field between the emitter and counter-electrode that causes Taylor-cone formation of liquid being dispersed. But where an array of emitters having proximal emitters that operate at the same time, their respective high-voltage potential affects the electric field, which interferes or even prevents Taylor-cone formation. The emitter interference impedes desired electrospray operation. The current subject matter includes a high-throughput adaptive electrospray device that rapidly switches the electrical potential of different emitters in an array on and off at a predetermined sequence that mitigates or eliminates interference from neighboring emitters.

In an aspect, an electrospraying apparatus includes a first current measuring unit, a second current measuring unit, and a controller. The first current measuring unit is electrically coupled to an emitter and measures an emitter current. The second current measuring unit is electrically coupled to a counter-electrode and measures a counter-electrode current. The controller is configured to receive an emitter current measurement and a counter-electrode current measurement; calculate, based on the received emitter current measurement and the received counter-electrode current measurement, a current adjustment value to compensate for parasitic current loss between the emitter and the counter-electrode; and adjust the emitter current based on the calculated current adjustment value.

In another aspect, an electrospraying apparatus includes an array of emitters and a controller. The array of emitters includes a first emitter and a second emitter. The controller is configured to energize the first emitter for a first period of time and to energize the second emitter for a second period of time. The first period of time and the second period of time are non-overlapping.

In yet another aspect, an emitter current measurement can be received from a first current measuring unit electrically coupled to an emitter and measuring an emitter current. A counter-electrode current measurement is received from a second current measuring unit electrically coupled to a counter-electrode and measuring a counter-electrode current. A current adjustment value is calculated based on the received emitter current measurement and the received counter-electrode current measurement. The current adjustment value is to compensate for parasitic current loss between the emitter and the counter-electrode. The emitter current can be adjusted based on the calculated current adjustment value.

One or more of the following features can be included in any feasible combination. For example, a current source can be electrically coupled to the emitter, the current source providing current at a voltage greater or less than 500 Volts relative to the counter-electrode. An array of emitters can include the emitter and a second emitter. The controller can be configured to energize the first emitter for a first period of time and to energize the second emitter for a second period of time. The first period of time and the second period of time can be non-overlapping.

A microfluidic solution source can be included and can be configured to provide solution continuously to the emitter. The first current measuring unit can include a high voltage nano-ammeter. The device can further include the emitter. The emitter can include a cannula for dispersing fluid. The device can further include the counter-electrode. The counter-electrode can be arranged to receive dispersed charged solution emitted by the emitter. The counter-electrode can include gold, Indium-tin-oxide (ITO), copper, nickel-plated copper, or stainless steel. The emitter can disperse or spray liquid into an environment having between 0.1 atmosphere and 10 atmosphere.

The device can further include a liquid source including a gravity reservoir. The device can include a liquid source including an electro-osmatic (EO) pump that has an electrical potential greater than the emitter. In some implementations, a constantly controlled pressure source can be included.

An extractor can be arranged between the emitter and the counter-electrode. The extractor can have an electric potential difference from the counter-electrode that is less than the electric potential difference between the emitter and the counter-electrode, the extractor including an adjustable annular aperture.

Calculating a current adjustment value can include subtracting the measured counter-electrode current from the measured emitter current. The second current measuring unit can be a current mirror. An emitter switch can couple the emitter to a power source and can receive a control signal. Adjusting the emitter current based on the calculated current adjustment value can include modifying a duty cycle of the control signal. The control signal can be pulse width modulated.

Each emitter in the array of emitters can have a corresponding counter-electrode. A microfluidic solution source can be configured to provide solution continuously to the array of emitters. A first electronic switch can control the first emitter. A second electronic switch can control the second emitter. The controller can energize the first emitter by providing a first control signal to the first electronic switch. The first control signal can be pulse width modulated and have a duty cycle.

The controller can be configured to: receive an emitter current measurement and a counter-electrode current measurement; calculate, based on the received emitter current measurement and the received counter-electrode current measurement, a current adjustment value to compensate for parasitic current loss between the emitter and the counter-electrode; and adjust the emitter current based on the calculated current adjustment value by modifying the duty cycle of the first control signal.

The duty cycle can be between 1 and 99 percent. The duty cycle can be about 10, 50, 70, or 90 percent, wherein about is within 10 percent. The duty cycle can be greater than 50 percent. The control signal can include a frequency between 1 Hertz and 10,000 Hertz. The frequency can be about 1, 100, or 1000 Hertz, wherein about is within 10 percent. A mixing element can be fluidically connected to the emitter, the mixing element for mixing polymer and cells prior to provision to the emitter for electrospraying.

An image acquisition device can be included and arranged to view a region between the emitter and the counter electrode, the image acquisition device configured to acquire an image of the region. The controller can be configured to, using the image of the region, detect a characteristic of a particle within the region. A rejection element can be included and can be coupled to the controller. The rejection element can reject a particle (e.g., emitter output) that does not satisfy a criterion by changing particle path from emitter to collection area. The rejection element can include an electrostatically charged element (e.g., and electrostatic scrubber), a pneumatic jet, a mechanical door, a shut off valve, and the like. The controller can be further configured to determine that the detected characteristic does not satisfy a criterion (e.g., exceeds a threshold value such as having physical dimensions outside a predetermined acceptable range) and, in response to the determination, actuate the electrostatic scrubber.

Solution can be sprayed, by the emitter, to form particles having a diameter between 10 nanometer and 3000 micrometers. The diameter can be between 1 micrometer and 2500 micrometers; between 1 micrometer and 100 micrometers; between 1 micrometer and 10 micrometers; between 10 micrometers and 50 micrometers; or between 20 micrometers and 40 micrometers.

Fabricating polymer-encapsulated living cells can include electrospraying a population of living cells and a polymer solution using the electrospraying apparatus. Living cells can be sprayed through a first emitter and the polymer solution can be sprayed through a second emitter.

A compound, therapeutic, or diagnostic can be mixed with a polymer. The mixing can occur in a mixing element fluidically connected to the first emitter and prior to provision to the first emitter for electrospraying.

In an aspect, an apparatus includes an electrospraying emitter; a first current measuring unit electrically coupled to the emitter and measuring an emitter current; a counter-electrode; a second current measuring unit electrically coupled to the counter-electrode and measuring a counter-electrode current; and a controller configured to: receive an emitter current measurement and a counter-electrode current measurement; calculate, based on the received emitter current measurement and the received counter-electrode current measurement, a current adjustment value to compensate for parasitic current loss between the emitter and the counter-electrode; and adjust the emitter current based on the calculated current adjustment value.

In some implementations, the apparatus further comprises a current source electrically coupled to the emitter, the current source providing current at a voltage greater or less than 500 Volts relative to the counter-electrode.

In some implementations, the apparatus further comprises an array of emitters including a first emitter and a second emitter, wherein the emitter is the first emitter; and the controller is configured to energize the first emitter for a first period of time and to energize the second emitter for a second period of time, the first period of time and the second period of time are non-overlapping.

In some implementations, the apparatus further comprises a microfluidic solution source configured to provide solution continuously to the emitter.

In some implementations, the first current measuring unit is a high voltage nano-ammeter.

In some implementations, the emitter includes a cannula for dispersing fluid.

In some implementations, the counter-electrode is arranged to receive dispersed charged solution emitted by the emitter.

In some implementations, the counter-electrode includes gold, Indium-tin-oxide (ITO), copper, nickel-plated copper, or stainless steel.

In some implementations, the emitter disperses liquid into an environment having between 0.1 atmosphere and 10 atmosphere.

In some implementations, the apparatus further comprises a liquid source including a gravity reservoir.

In some implementations, the apparatus further comprises a liquid source including an electro-osmatic (EO) pump that has an electrical potential greater than the emitter.

In some implementations, the apparatus further comprises an extractor arranged between the emitter and the counter-electrode, the extractor having an electric potential difference from the counter-electrode that is less than the electric potential difference between the emitter and the counter-electrode, the extractor including an adjustable annular aperture.

In some implementations, calculating a current adjustment value comprises: subtracting the measured counter-electrode current from the measured emitter current.

In some implementations, second current measuring unit is a current mirror.

In some implementations, the apparatus further comprises an emitter switch coupling the emitter to a power source and receiving a control signal; and adjusting the emitter current based on the calculated current adjustment value includes modifying a duty cycle of the control signal, the control signal pulse width modulated.

In some implementations, the duty cycle is between 1 and 99 percent.

In some implementations, the duty cycle is about 10, 50, 70, or 90 percent, wherein about is within 10 percent.

In some implementations, the control signal includes a frequency between 1 Hertz and 10,000 Hertz.

In some implementations, the frequency is about 1, 100, or 1000 Hertz, wherein about is within 10 percent.

In some implementations, the apparatus further comprises a mixing element fluidically connected to the emitter, the mixing element for mixing polymer and cells prior to provision to the emitter for electrospraying.

In some implementations, the apparatus further comprises an image acquisition device arranged to view a region between the emitter and the counter electrode, the image acquisition device configured to acquire an image of the region; and the controller is configured to, using the image of the region, detect a characteristic of a particle within the region.

In some implementations, the apparatus further comprises a rejection element operatively coupled to the controller, wherein the controller is further configured to determine that the detected characteristic does not satisfy a criterion and, in response to the determination, actuate the rejection element, wherein the rejection element is an electrostatic deflection element, an air jet, a mechanical door, or a shut off valve.

In another aspect, an apparatus comprising: an array of electrospraying emitters including a first emitter and a second emitter; and a controller configured to energize the first emitter for a first period of time and to energize the second emitter for a second period of time, wherein the first period of time and the second period of time are non-overlapping.

In some implementations, each emitter in the array of emitters has a corresponding counter-electrode.

In some implementations, the apparatus further comprises a microfluidic solution source configured to provide solution continuously to the array of emitters.

In some implementations, the apparatus further comprises a first electronic switch controlling the first emitter; and a second electronic switch controlling the second emitter.

In some implementations, the controller energizes the first emitter by providing a first control signal to the first electronic switch, the first control signal pulse width modulated and having a duty cycle.

In some implementations, the controller is further configured to: receive an emitter current measurement and a counter-electrode current measurement; calculate, based on the received emitter current measurement and the received counter-electrode current measurement, a current adjustment value to compensate for parasitic current loss between the emitter and the counter-electrode; and adjust the emitter current based on the calculated current adjustment value by modifying the duty cycle, a voltage, or a frequency of the first control signal.

In some implementations, the duty cycle is greater than 50 percent.

In some implementations, the duty cycle is between 1 and 99 percent.

In some implementations, the duty cycle is about 70, or 90 percent, wherein about is within 10 percent.

In some implementations, the control signal includes a frequency between 1 Hertz and 10,000 Hertz.

In some implementations, the frequency is about 1, 100, or 1000 Hertz, wherein about is within 10 percent.

In some implementations, the apparatus further comprises a mixing element fluidically connected to the emitter, the mixing element for mixing polymer and cells prior to provision to the emitter for electrospraying.

In some implementations, the apparatus further comprises an image acquisition device arranged to view a region between the emitter and the counter electrode, the image acquisition device configured to acquire an image of the region; and the controller is configured to, using the image of the region, detect a characteristic of a particle within the region.

In some implementations, the apparatus further comprises a rejection element operatively coupled to the controller, wherein the controller is further configured to determine that the detected characteristic does not satisfy a criterion and, in response to the determination, actuate the rejection element, wherein the rejection element is an electrostatic deflection element, an air jet, a mechanical door, or a shut off valve.

In yet another aspect, a method comprises: receiving, from a first current measuring unit electrically coupled to an emitter and measuring an emitter current, an emitter current measurement; receiving, from a second current measuring unit electrically coupled to a counter-electrode and measuring a counter-electrode current, a counter-electrode current measurement; calculating, based on the received emitter current measurement and the received counter-electrode current measurement, a current adjustment value to compensate for parasitic current loss between the emitter and the counter-electrode; and adjusting the emitter current based on the calculated current adjustment value.

In some implementations, the first current measuring unit is a high voltage nano-ammeter.

In some implementations, the emitter includes a cannula for dispersing fluid.

In some implementations, the counter-electrode is arranged to receive dispersed charged solution emitted by the emitter.

In some implementations, the method further comprises spraying, by the emitter, solution into an environment having between 0.1 atmosphere and 10 atmosphere.

In some implementations, calculating a current adjustment value comprises: subtracting the measured counter-electrode current from the measured emitter current.

In some implementations, adjusting the emitter current based on the calculated current adjustment value includes modifying a duty cycle of a control signal, the control signal pulse width modulated and controlling an emitter switch coupling the emitter to a power source.

In some implementations, the duty cycle is greater than 50 percent.

In some implementations, the duty cycle is about 70 or 90 percent, wherein about is within 10 percent.

In some implementations, the control signal includes a frequency between 1 Hertz and 10,000 Hertz.

In some implementations, the frequency is about 1, 100, or 1000 Hertz, wherein about is within 10 percent.

In some implementations, the apparatus further comprises a mixing element fluidically connected to the emitter, the mixing element for mixing polymer and cells prior to provision to the emitter for electrospraying.

In some implementations, the apparatus further comprises an image acquisition device arranged to view a region between the emitter and the counter electrode, the image acquisition device configured to acquire an image of the region; and the controller is configured to, using the image of the region, detect a characteristic of a particle within the region.

In some implementations, the apparatus further comprises a rejection element operatively coupled to the controller, wherein the controller is further configured to determine that the detected characteristic does not satisfy a criterion and, in response to the determination, actuate the rejection element, wherein the rejection element is an electrostatic deflection element, an air jet, a mechanical door, or a shut off valve.

In some implementations, the method further comprises spraying, by the emitter, solution to form particles having a diameter between 10 nanometer and 3000 micrometers.

In some implementations, the diameter is between 1 micrometer and 2500 micrometers; between 1 micrometer and 100 micrometers; between 1 micrometer and 10 micrometers; between 10 micrometers and 50 micrometers; or between 20 micrometers and 40 micrometers.

In some implementations, a method of fabricating polymer-encapsulated living cells comprises electrospraying a population of living cells and a polymer solution using the apparatus.

In some implementations, the living cells are sprayed through a first emitter, and the polymer solution is sprayed through a second emitter.

In some implementations, the method further comprises: mixing a compound, therapeutic, or diagnostic with a polymer, the mixing occurring in a mixing element fluidically connected to the first emitter and prior to provision to the first emitter for electrospraying.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a system block diagram illustrating an adaptive electrospray device;

FIG. 2 is an illustration of an example electrospray device;

FIGS. 3-5 are photographs of the example implementation;

FIGS. 6-10 illustrate an example housing and reservoir configuration that provides a steady, tubeless and pulseless input of solution;

FIG. 11 illustrates an example extractor;

FIG. 12 illustrates the arrangement of the example extractor of FIG. 11 between emitter and counter-electrode;

FIG. 13 is an electrical circuit model of an electrospray device;

FIG. 14-16 illustrate a current regulated power supply and the example system connected to a current regulated power supply;

FIG. 17 is a process flow diagram illustrating a method for adapting an electrospray system (for example, the electrospray system illustrated in FIGS. 1 and 2) to compensate for variable humidity;

FIG. 18 illustrates an emitter array with emitters spaced in a circular arrangement;

FIG. 19-21 illustrate the voltage and field strength distribution for three emitter operating scenarios;

FIGS. 22A-B and 23A-B illustrate four example emitter array arrangements;

FIG. 24 is a photograph of a single use sterile bag for GMP cell processing and manufacture;

FIG. 25 is a system block diagram of an example implementation of a current control module;

FIG. 26 is a system block diagram of another example implementation of a current control module;

FIG. 27 is a emitter excitation timing diagram illustrating example control signals to selectively activate emitters in an array of N emitters;

FIG. 28 is an emitter excitation timing diagram illustrating pulse width modulation of a single control signal pulse;

FIG. 29 shows the relationship between current and voltage for Phosphate-buffered saline (PBS) 1X as measured using an example implementation of a electrospray device;

FIG. 30 illustrates the output of an emitter at varying voltages;

FIG. 31-32 illustrate images of an example particle sizing and tracking algorithm;

FIGS. 33 and 34 illustrate the example microfluidic mixing chip;

FIG. 35 illustrates percentage of living human T lymphocytes (Jurkat) cells after mixing in the example Dolomite Microfluidic mixer chip;

FIG. 36-37 illustrates example control signals where pulse width (PW) is the positive pulse active time and T is the period of the signal;

FIG. 38 shows a series of image captures of an electrospray process in which the emitter is constantly energized;

FIG. 39-52 illustrate emitter output for an example electrospray device for different control signals and solutions;

FIG. 53 illustrates an alginate electrospray control at 5.4 KV, with monochrome images taken for 66 mS snapshots over 2 seconds;

FIG. 54-74 are images of an emitter output for an example electrospray device for different control signals and encapsulation solutions;

FIG. 75-80 illustrate light microscope images of particles electrosprayed with different encapsulating solutions and control signals;

FIG. 81-85 illustrate images of an example electrospray of different solutions within a climate control chamber at different temperatures and humidity;

FIG. 86 illustrates an emitter coated with Fluorinated ethylene propylene (FEP);

FIG. 87 illustrates several images of an example nano-ammeter; and

FIG. 88 is an example implementation of an electrospray device illustrating aspects of an automatic deflection capabilities in which particles are rejected based on size, morphology and/or content.

Like reference symbols in the various drawings indicate like elements.

### DETAILED DESCRIPTION

The current subject matter includes an adaptive electrospray device that creates consistent output when operating in atmosphere (e.g., not a vacuum). Because electrospray output depends on current flowing from an emitter to a counter-electrode (also referred to as a collector), ambient humidity causes parasitic current losses. The ionization current is not routinely used as a characterization parameter. The current subject matter includes devices that use ionization current as a characterization parameter of operation. For example, the current subject matter includes an adaptive system that can monitor two current reference points (at the emitter and counter-electrode, respectively), determine a change in emitter current that will account for the parasitic losses, and adjust the emitter current accordingly. This approach enables the system to operate independently of ambient conditions, such as humidity, by adjusting other components to compensate based on a current control signal. Further, by monitoring current at both the emitter and the counter-electrode, the accuracy of the control system is improved.

In addition, the current subject matter includes adaptive electrospray devices having an array of emitters. Where a single emitter/counter-electrode pair are used, the electric potential difference between the emitter and counter-electrode create an electric field between the emitter and counter-electrode that causes Taylor-cone formation of liquid being dispersed. But where an array of emitters having proximal emitters that operate at the same time, their respective high-voltage potential affects the electric field, which interferes or even prevents Taylor-cone formation. The emitter interference impedes desired electrospray operation. The current subject matter includes a high-throughput adaptive electrospray device that rapidly switches the electrical potential of different emitters in an array on and off at a predetermined sequence that mitigates or eliminates interference from neighboring emitters. For example, in one implementation, each emitter in an array is operated for 1 millisecond and at rest for 9 milliseconds such that Taylor-cone formation is maintained but emitter interference is reduced.

An example implementation of the current subject matter includes a device with a consistent output that is capable of producing throughput material that is homogeneous, can maintain a particular character over extended periods of time (hours, days, weeks, months), minimizes electrospraying commencement artefacts, produces the same character on each power-up of the system and renders the process independent of humidity within a range of 20%-60% relative humidity. Relative humidity is the ratio of the partial pressure of water vapor to the equilibrium vapor pressure of water at a given temperature. Relative humidity depends on the temperature and the pressure of the system of interest.

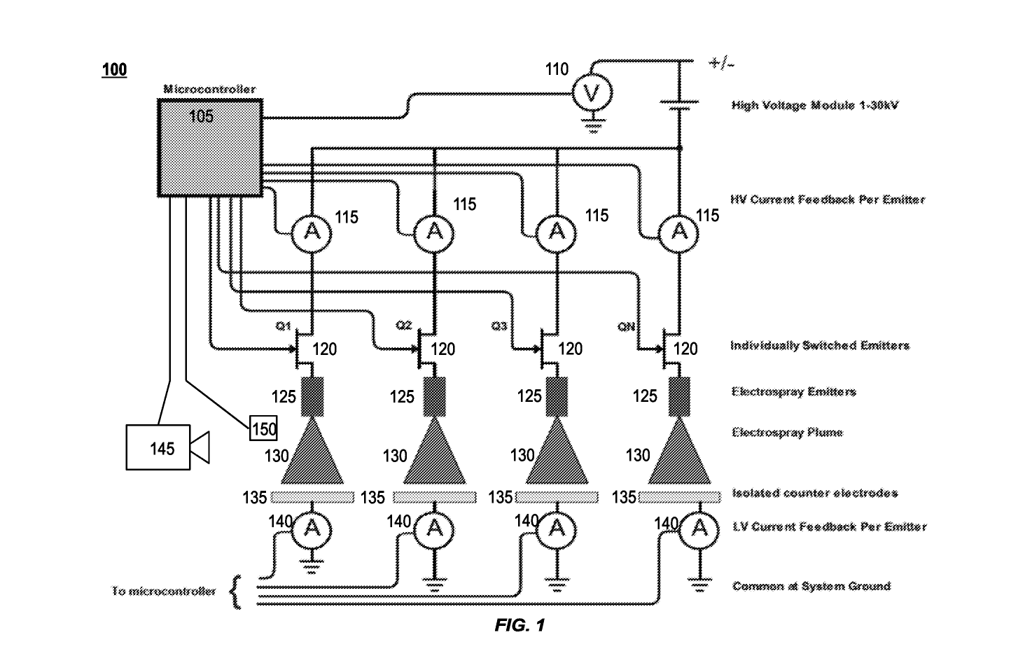

FIG. 1 is a system block diagram illustrating an adaptive electrospray device 100. The adaptive electrospray device includes a controller 105, a high-voltage module 110, one or more high-voltage current feedback sensors 115, one or more emitter control switches 120, one or more electrospray emitters 125, one or more isolated counter electrodes 135 and one or more low voltage current feedback sensor 140.

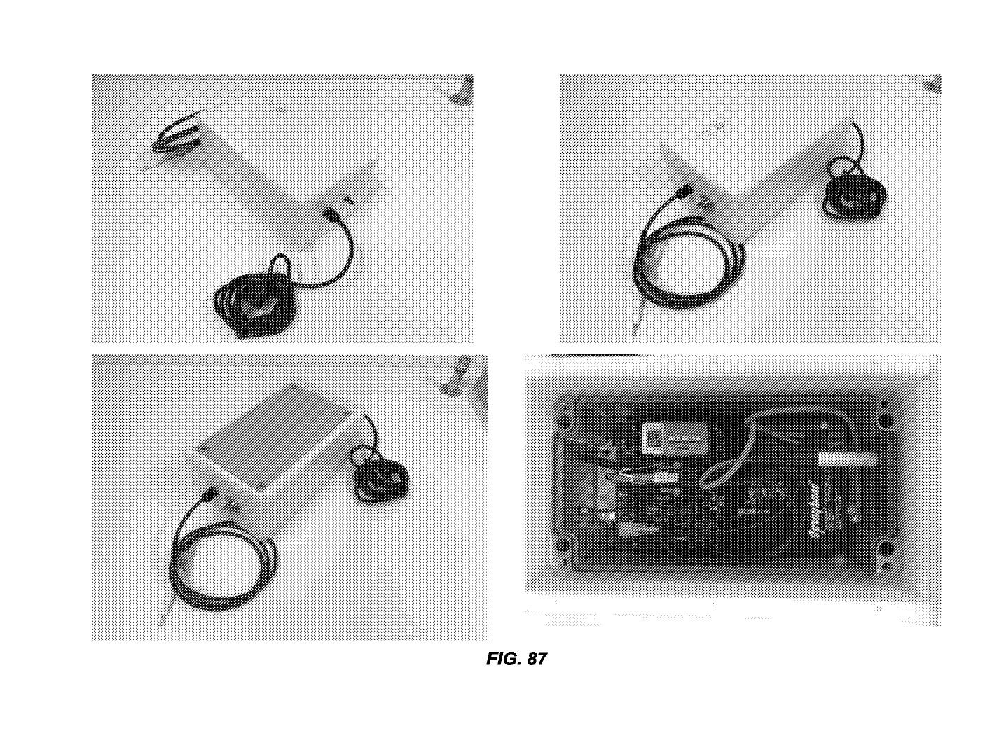

The high-voltage current feedback sensor can include a current measuring unit electrically coupled to an associated emitter 125 and measuring an emitter current. The high voltage current sensor can be galvanically isolated from the measurement circuitry and/or controller. This can be achieved using optical isolation. An example high-voltage current feedback sensor can include a high voltage nano-ammeter, (Sauer, B. E., Kara, D. M., Hudson, J. J., Tarbutt, M. R. and Hinds, E. A., 2008. A robust floating nano-ammeter. Review of Scientific Instruments, 79(12), p. 126102). FIG. 87 illustrates several images of an example nano-ammeter.

The low-voltage current feedback sensor can include a current measuring unit electrically coupled to an associated counter-electrode 135 and measuring a counter-electrode current. While FIG. 1 is illustrated with four emitters, it is understood that the number of emitters can be greater or less than 4, for example, there may be N emitters.

In some implementations, the adaptive electrospray device 100 can include an image acquisition device 145 communicatively coupled to the controller 105 and arranged to image the region between one or more of the emitters 125 and respective counter electrodes 135. The image acquisition device 145 can be utilized to capture images of particles. Based on those images, the particles can be analyzed to determine characteristics of the particle, such as size, morphology, content, and the like. The determined characteristic can be used as feedback to adjust the operating parameters of the adaptive electrospray device, for example, changing the voltage, duty cycle, frequency, solution input pressure, and the like. The determined characteristic can be used in combination with a rejection element to reject particles that do not satisfy a criterion, such as when a particle diameter is outside a predetermined range (e.g., the particle is too large or too small).

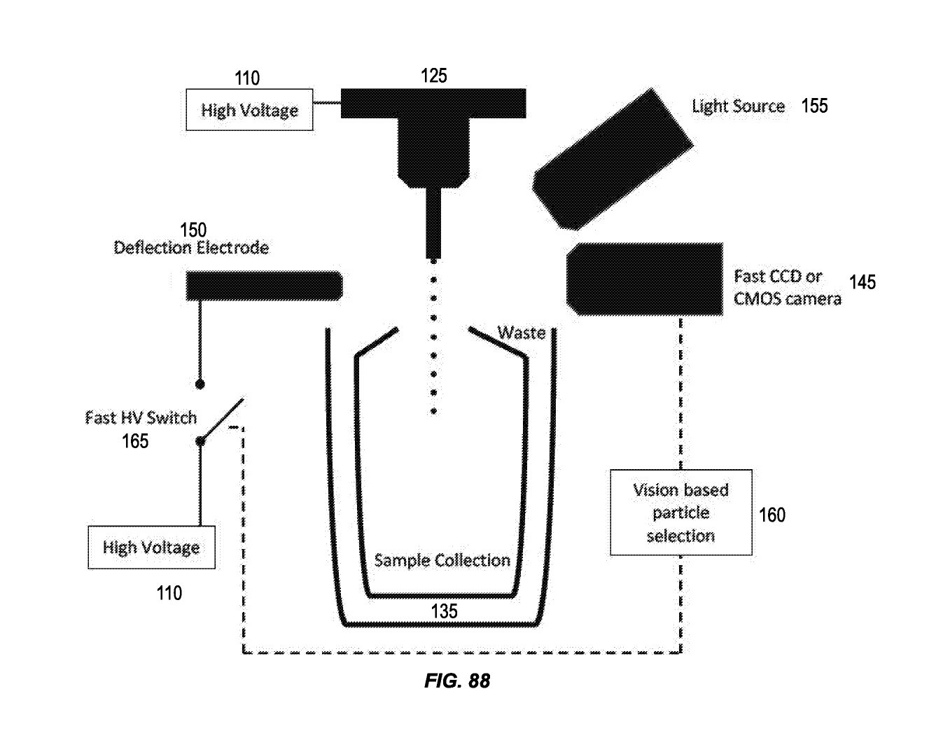

FIG. 88 is an example implementation of an electrospray device illustrating aspects of an automatic deflection capabilities in which particles can be rejected based on size, morphology and/or content. A light source 155 illuminates the output of emitters 125 and the image acquisition device 145 can capture images of the electrospray plume or particles (e.g., droplets) created by the electrospray. A vision based particle selection module 160 (which can be implemented with controller 105) can analyze the images to determine characteristics of the plume or particles in real time. In some implementations, the adaptive electrospray device 100 can include a chargeable deflection element 150 (e.g., a charged scrubber, electrode, plate, and the like) that, when energized (e.g., to a polarity opposite or the same as that of the emitters 125), can alter the flight path of a particle as the particle travels from the emitters 125 to the counter electrodes 135. In some implementations, the vision based particle selection module 160 can assess particles formed by emitters 125 and, if they do not meet certain criteria (e.g., size, morphology, composition, and the like), the vision based particle selection module 160 can activate the chargeable deflection element 150 using a fast high voltage switch 165 to alter the flight path of the particle so that the particle does not land on the collection area (e.g., counter electrode 135) or associated collection point. As illustrated in FIG. 88, the sample collection area can include a waste collection area for rejected particles. In some implementations, the vision based particle selection module 160 is controller 105.

Although the example rejection element is described as a deflection element, other implementations are possible. For example, the adaptive electrospray device can include a rejection element that can include an electrostatically charged element (e.g., and electrostatic scrubber), a pneumatic jet, a mechanical door, a shut off valve, and the like.

In some implementations, the adaptive electrospray device 100 can include and/or reside in a closed temperature and humidity chamber. In some implementations, the controller 105 can be operatively and communicatively coupled to the temperature and humidity chamber. In some implementations, the controller 105 can adjust or control the temperature and humidity within the chamber to alter characteristics, such as morphology, of the electrosprayed materials. Because the adaptive electrospray device can compensate for parasitic current loss to humidity, controlling humidity can be a controllable approach to adjusting some characteristics of electrosprayed materials.

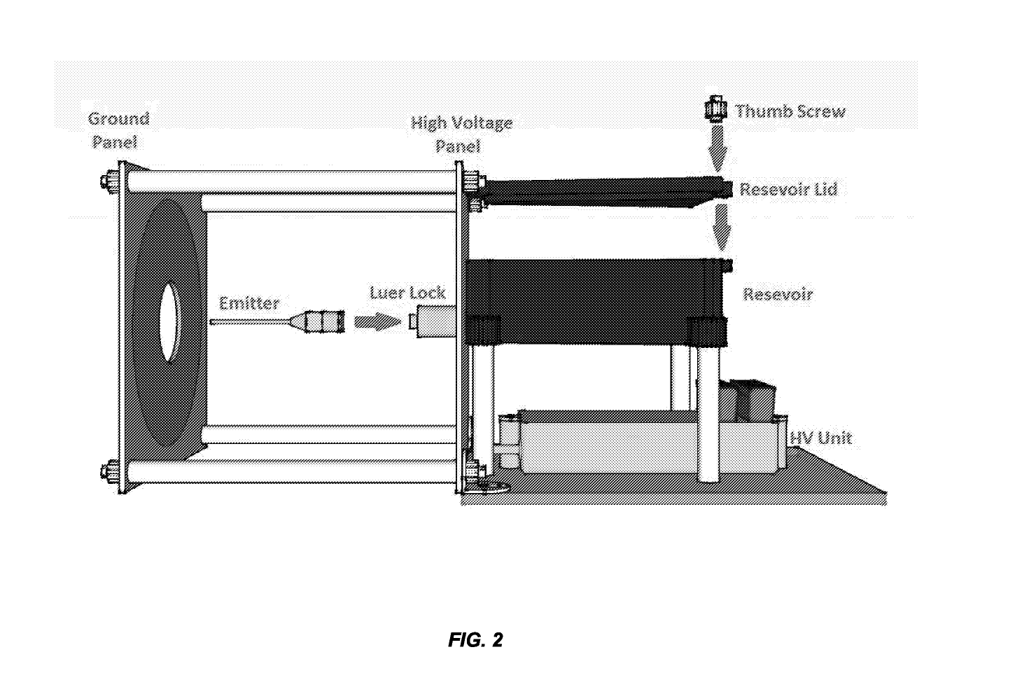

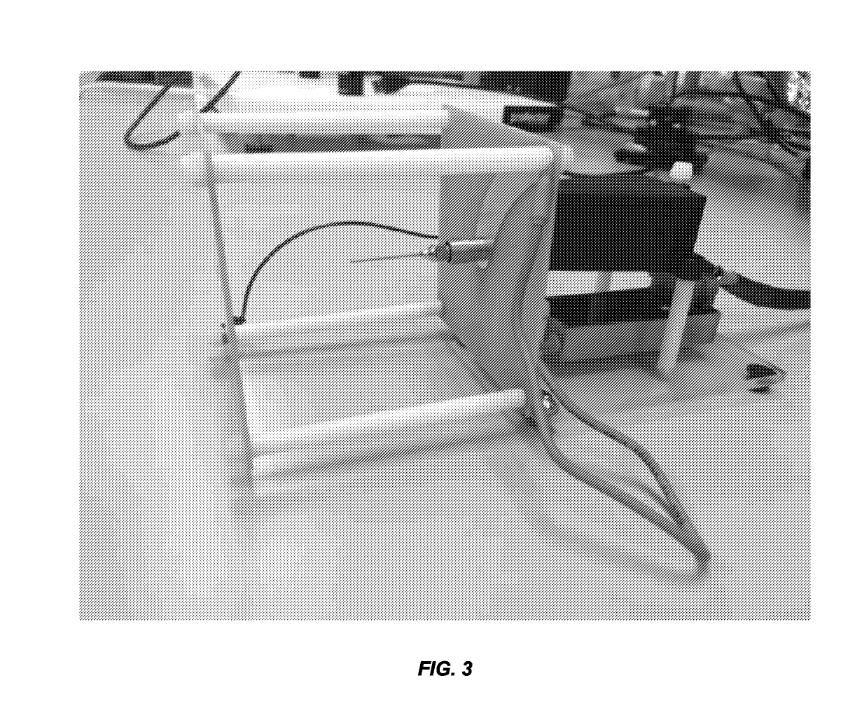

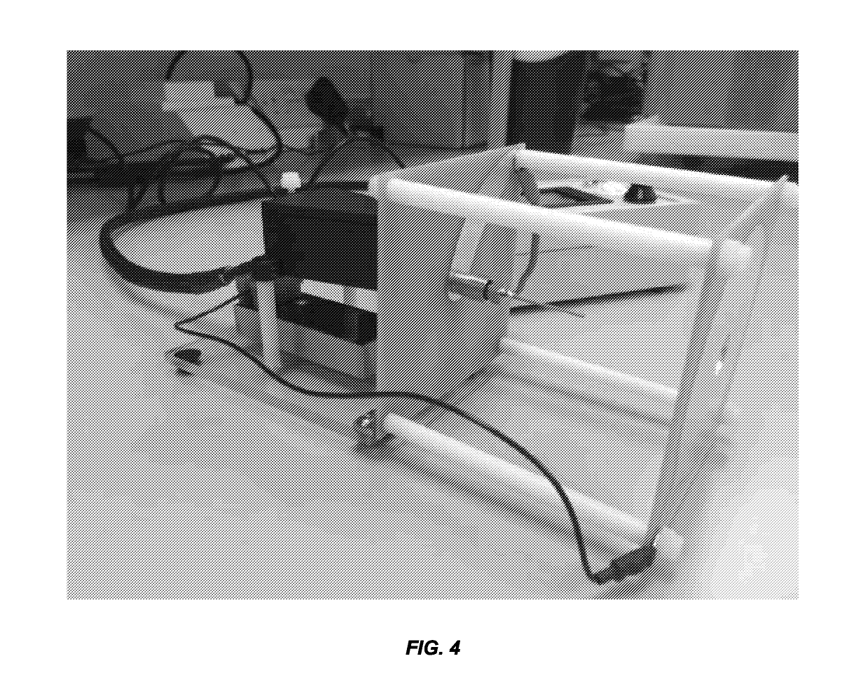

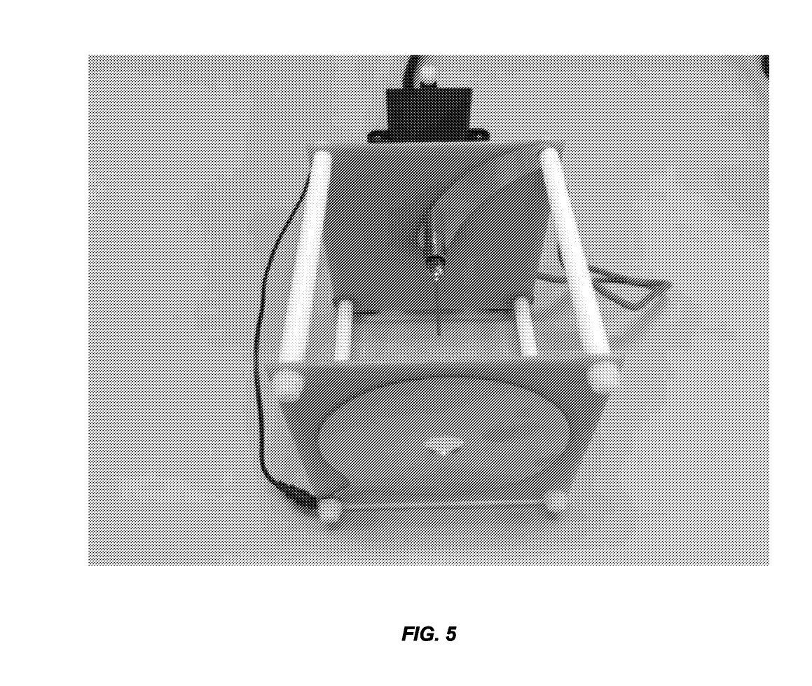

FIG. 2 is an illustration of an example electrospray device and FIGS. 3-5 are photographs of the example implementation. The example electrospray device includes a high-voltage panel, a ground panel with a counter-electrode, an emitter which is connected to a reservoir, a high-voltage power unit, and a controller.

The high-voltage power supply can include a high-voltage DC-DC biasing supply capable of providing a wide range of power from 0V to 25 kV supply at up to 30 watts of output power. The high-voltage power supply can be provided in a format for printed circuit board applications. For example, the power supply can include the 10A-24ADS supply manufactured by ULTRAVOLT®, Ronkonkoma, N.Y.

The example device generates an electrospray by delivering a chemical composition in solution form to the end of a cannula which is raised to a high electrical potential, positive or negative above a counter electrode. The charged cannula (the emitter) is physically separated from the collector plate by some distance. The medium in which the system operates presents a resistance between the emitter and the collector and the voltage drop across this resistance then generates a current which is typically in the 5 nA-5000 nA range for a mono-disperse electrospray. Usually, the emitter is held at a high electrical potential above the collector surface which is usually, but not always, grounded. It is also possible to have an extractor (e.g., a flat, annular surface held at an intermediate voltage between that of the emitter and that of the collector) in the path of the spray in_order to manipulate its focus on the collector.

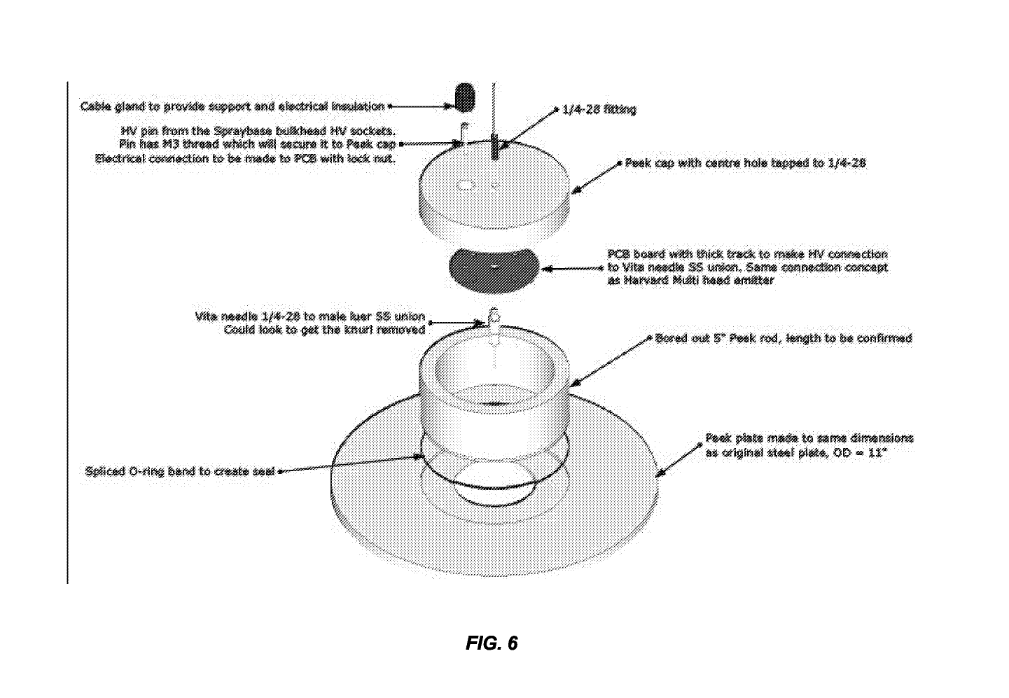

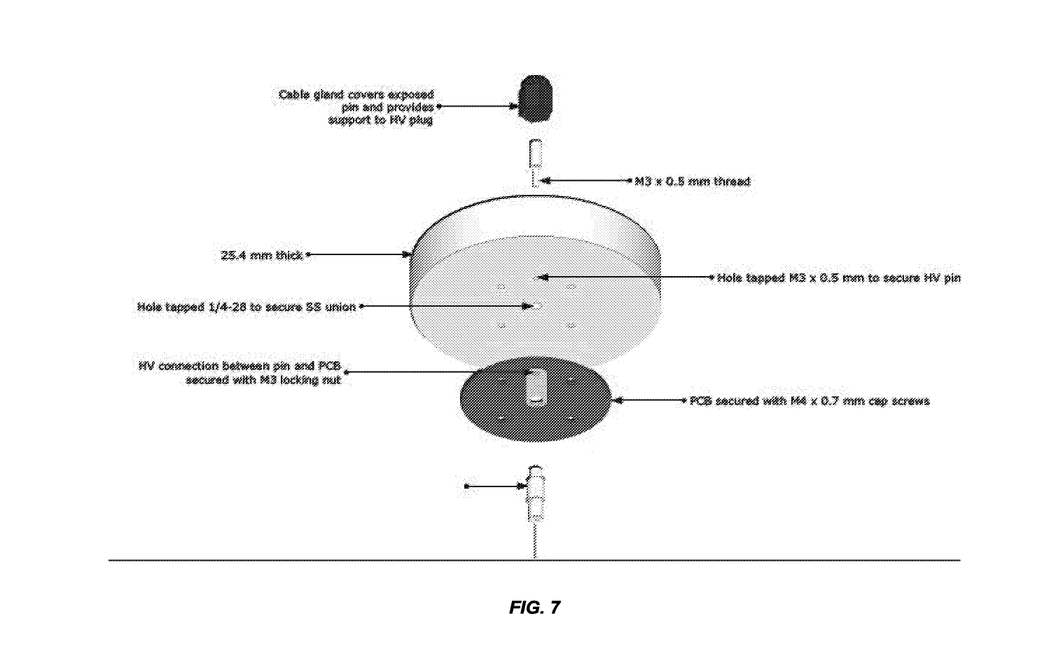

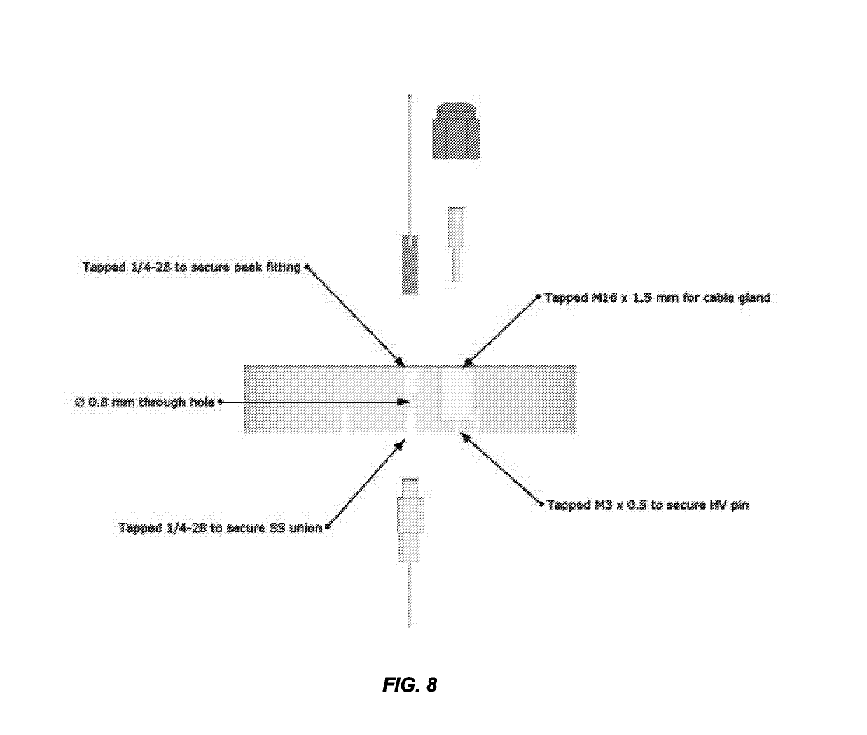

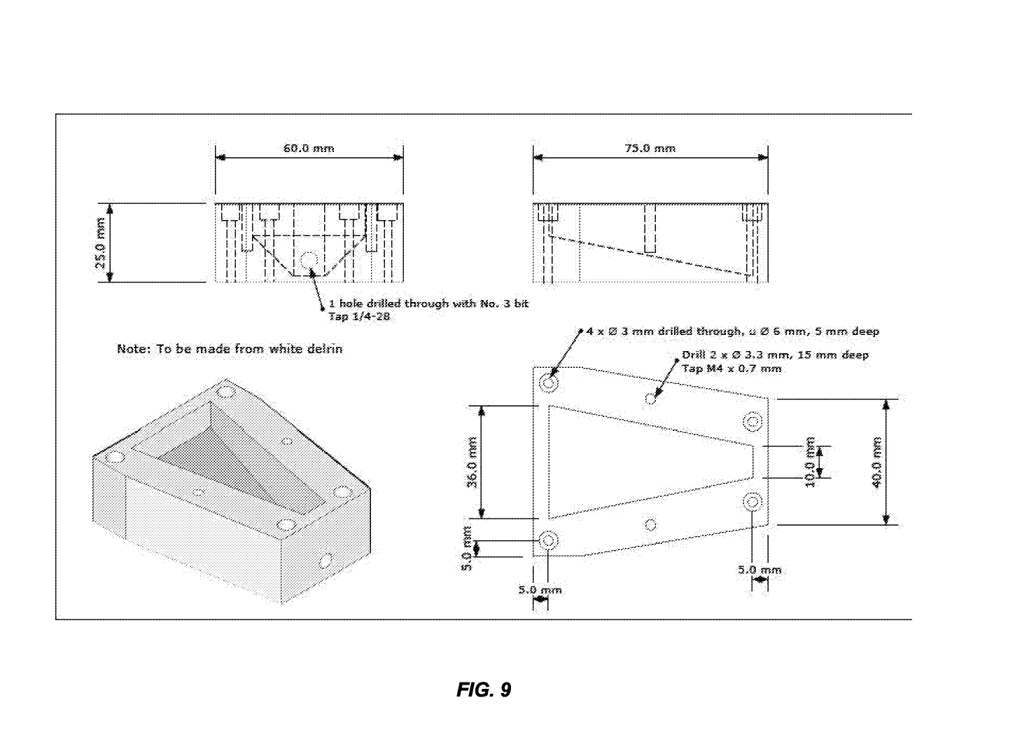

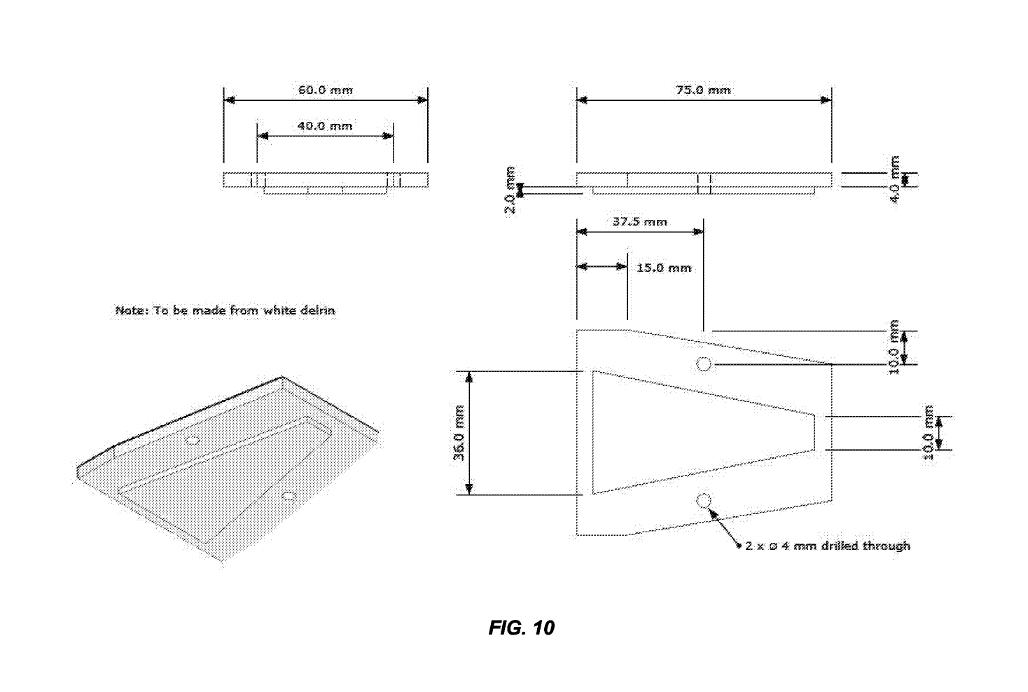

For delivering solution to the emitter, the pumping means can be located close to the emitter, if not substantially part of it. For example, an example implementation of the current subject matter includes a combined electrospray emitter pump. An electro-osmatic (EO) pump may be connected to an emitter electrically isolating the EO pump such that it electrically floats at a potential above the electrospray potential. Alternatively or in addition, a gravity or constant pressure head applied to a fluidic reservoir can feed a microfluidic distributer chip such that a number of equal outputs can be derived from a steady, pulseless input. Each of the outputs can feed an individual emitter. In some implementations, the current subject matter uses constant flow methods rather than a syringe pump to deliver a steady, pulseless flow of water to the charged emitter to generate an electrospray with consistent output. FIGS. 6-10 illustrate an example housing and reservoir configuration that provides a steady, pulseless input of solution. This housing may contain porous material such as sponge material or glass beads to regulate fluid flow depending on the fluid solution character.

In some implementations, a microfluidic mixing chip can be included to combine one or more solutions and/or to ensure homogenous mixing of a cell-polymer solution prior to entering the high-voltage emitter.

In the example implementation, the emitter can attach to a first printed circuit board by, for example, a luer lock. The counter electrode is attached to a second printed circuit board, where the circuit board is between the counter electrode and the system ground. On the second board, the counter electrode is etched and flashed in gold. The counter electrode can include gold, Indium-tin-oxide (ITO), or stainless steel. For a EWNS (Engineered Water Nano Structures) application, the shape of the counter electrode can be annulus. The gold surface finish ensures that the counter electrode does not oxidize or reduce in operation. Non-oxidized counter electrodes promote stable electrospray output. The size of the counter electrode and size of the annular hole including gold plating of inner circumference ensures a stable geometry.

The boards are opposing, and the distance between the emitter and the counter electrode can be controlled by spacers that are between the boards, precision machined for accuracy. The distance between the emitter and the counter electrode can be chosen for a particular solution. In some implementations, the housing can provide for telescopic control of the distance between the emitter and the counter electrode to allow for electrode separation as a control parameter input.

The opposing boards have electrically, individually addressable emitter positions axially aligned with the counter electrode hole. In each case the circuit board have circuitry enabling the measurement of potential difference and current through that emitter.

Voltage potential is frequently used to control electrospray output. It is also known to use electrospray current as a control means (Gamero-Casta and Hruby, “Electric Measurements of Charged Sprays Emitted by Cone-Jets” J. Fluid Mech (2002), vol. 459, pp. 245-276; Marsh et al, “The Control of Electrostatic Atomization Using a Closed-Loop System” Journal of Electrostatics, 20 (1988) 313-318). The problem with using electrospray current is that in atmospheric electrospraying, current is consumed parasitically through ionisation current losses to the air. By measuring the currents at the first and second circuit boards, it is possible to subtract the current at the second board, I3, from the current at the first board, I1, to calculate the losses due to atmospheric ionization, I2. Such a system obeys Kirchhoff's Law.

In some implementations, the current through each board is monitored using current meters. An electrospray generates a specific total current in the system, I1, which is comprised of the ionization current, I2, and the electrospraying current, I3.

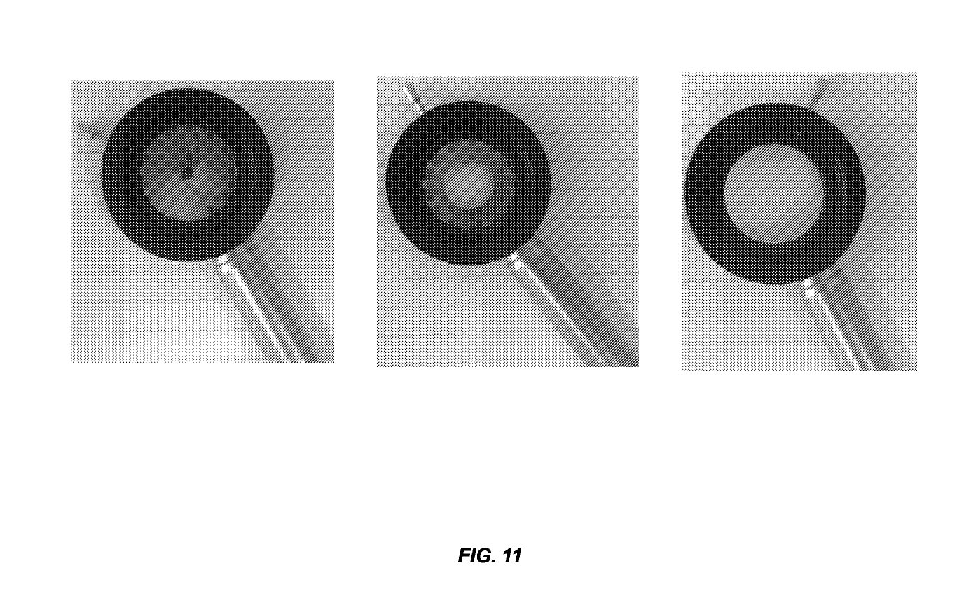

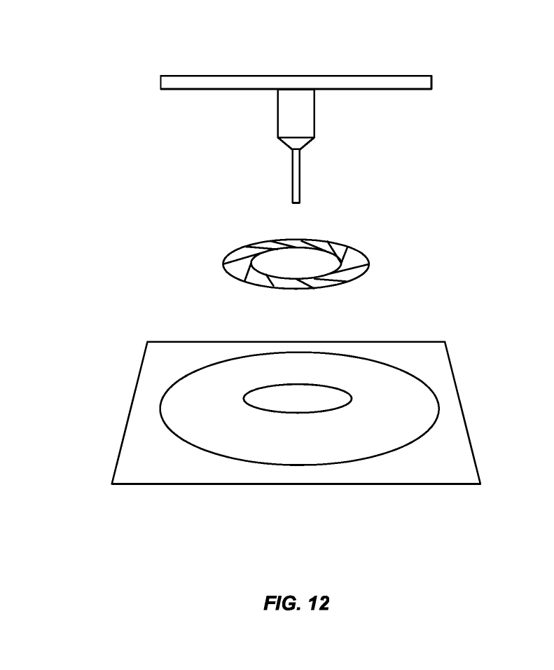

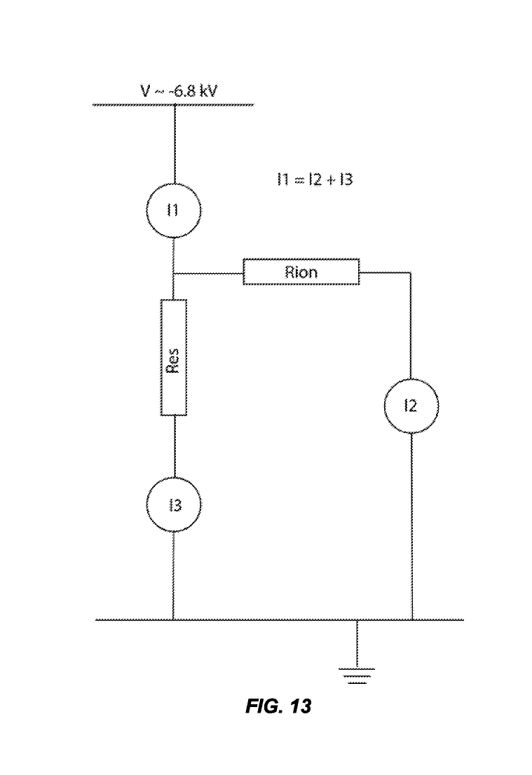

The shape of the electrical field can be manipulated using a third, intermediate counter electrode at a potential difference less that the emitter but higher than the counter electrode. This intermediate electrode can be annulus shape and may be called an extractor. It is physically located between the emitter and the counter electrode. The size of the annular ring (e.g., the size of the aperture) in the extractor can be controlled using an optical iris. An example extractor is illustrated in FIG. 11, and its arrangement between emitter and counter-electrode is illustrated in FIG. 12. Size of aperture can be used as a control parameter input. The extractor voltage is between the emitter and counter electrode. A typical or practical example can include the counter electrode at 0% of potential, emitter at 100% of a maximum potential and extractor at 85% of a maximum potential. Changes in the aperture size cause changes in the electrostatic field between emitter and counter electrode. This in turn modifies the pathway of charge particles travelling in the field and thus the spray pattern.

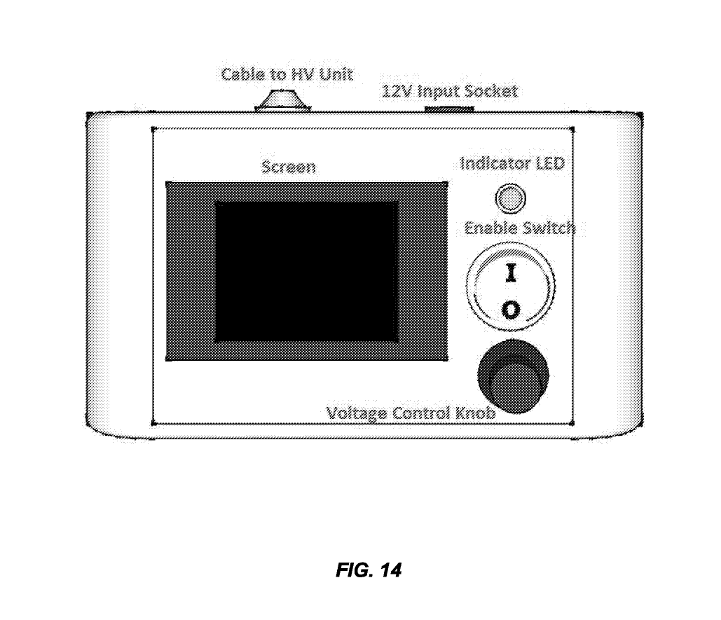

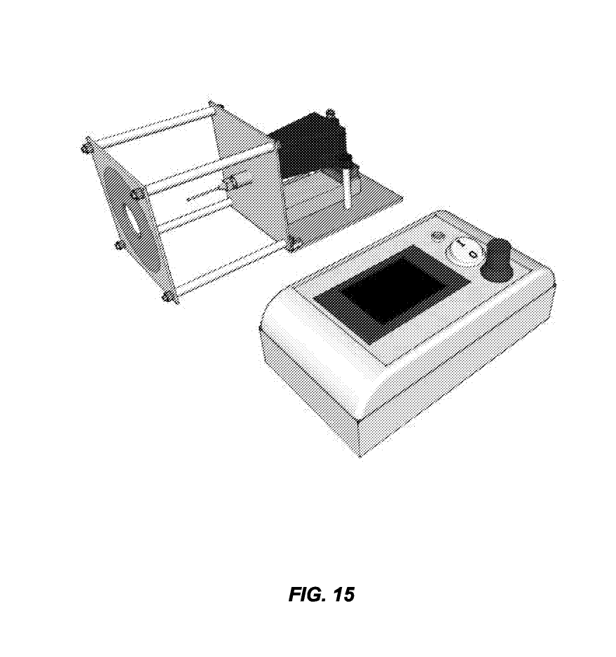

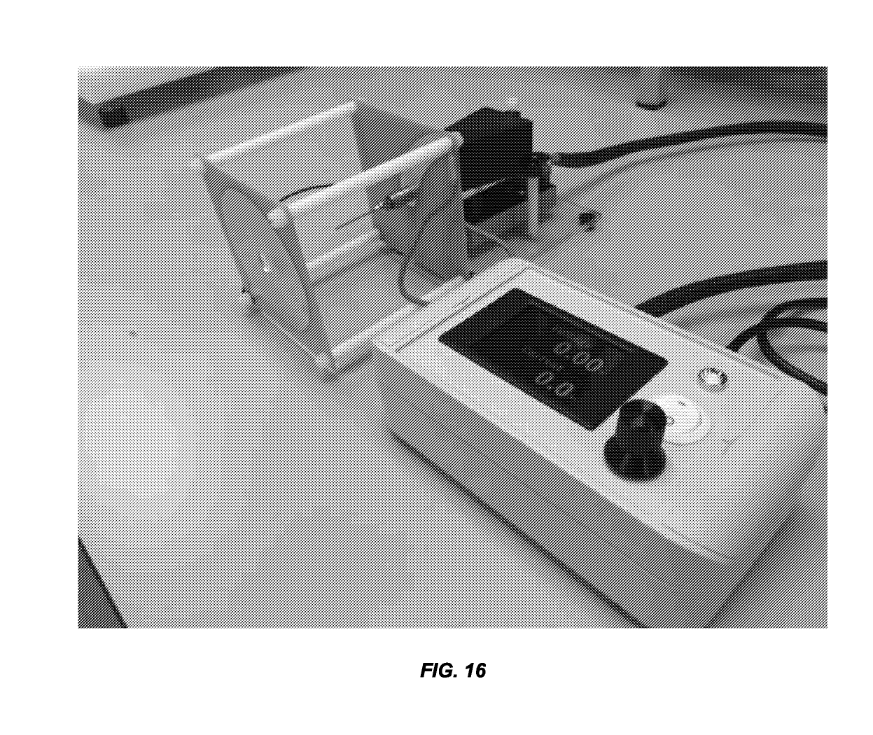

In some implementations, the current subject matter implements current manipulation to account for the medium's temporal variation in resistance as well as other parameters such as flow rate. Current manipulation is appropriate for generating an electrospray with a particular output and can vary the potential difference necessary to develop and retain that output, the value of which will fluctuate depending on the humidity. An advantage of manipulating current is that changes in the current circulating through the system (that is the current generated by the particles travelling between the charged cannula and the current lost to ionization) due to the medium's resistance variation is reduced or eliminated. The resistance presented to the system is a function of humidity, and is depicted in FIG. I3, by measuring I1 (the current at the emitter) and I3 (the current at the counter-electrode), the ionization current, I2 can be determined and mitigated for. I1 can be adjusted such that a desired I3 value is achieved. The desired I3 value may be provided or determined based on the particular solution or use of the electrospray device, for example, may vary based on the product being produced by the electrospray device. Modifying I1 to achieve a desired I3 creates the necessary current to generate an electrospray with a consistent output. This current adjustment procedure may be performed as a calibration procedure at regular intervals (e.g., once a day or at device startup) or continually, e.g., as a feedback loop. This regime mitigates the effect of environmental variation to create a consistent output electrospray device. FIG. 14-16 illustrate a current regulated power supply and the example system connected to a current regulated power supply.

Modification of current can be direct, such as using a current source, or achieved through other means including modifying a signal duty cycle, frequency, and voltage.

In some implementations, the energized state of the emitter (e.g., on or off) can be controlled using a digital pulsed signal which has a certain period and duty cycle (D). The controller can include at least two analog input channels and a microprocessor. At least 1 analog channel can record the emitter current through a high voltage nanoammeter. At least 1 other analog channel can record the collector current through a current mirror. Using a microprocessor, the analog input voltages can be processed and mathematical operations can be conducted on the signals to determine their difference, which can represent parasitic loss of current to the atmosphere. This value can be used to adjust the high voltage emitter current up or down to account for the parasitic losses.

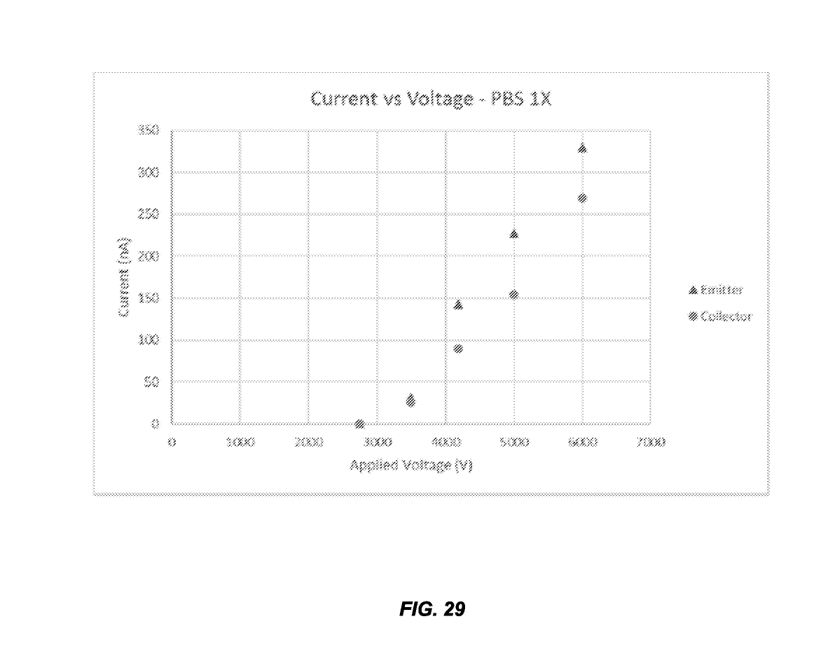

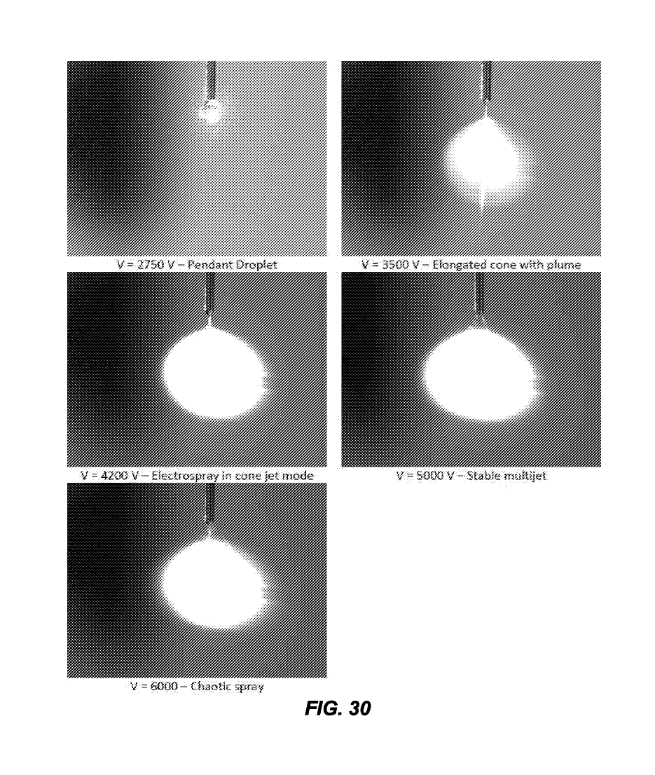

FIG. 29 shows the relationship between current and voltage for Phosphate-buffered saline (PBS) 1X as measured using an example implementation of an electrospray device. As can be seen, increasing the voltage increases the emitter and collector currents linearly. An electrospray process can be considered a resistive circuit when activated and obeys Ohm's law. As can also be seen from FIG. 29, the emitter current is consistently higher than the collector current when an electrospray has been fully formed. This difference represents the parasitic losses to the atmosphere in the system, for example as illustrated in FIG. I3 where I1=I2+I3. It has been shown that increasing the duty cycle of the digital control signal can have an equivalent effect on the process as increasing the emitter voltage when in a constantly energized state. As can be seem from FIG. 29 and FIG. 30, which illustrates the output of an emitter at varying voltages, varying the voltage varies the current draw in the system and in turn affects the resulting process. Hence, varying the duty cycle D of the process can also vary the current in the system and so can be used as effective control parameter.

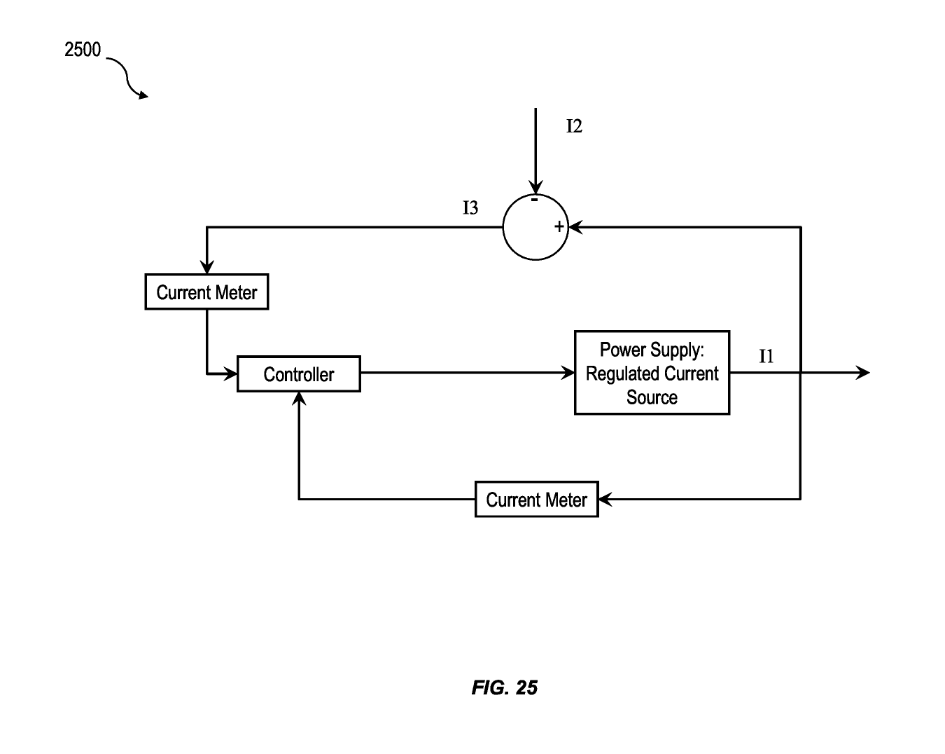

FIG. 25 is a system block diagram of an example implementation of a current control module 2500 (e.g., as implemented by a controller). The process begins by receiving a desired electrospray current value (e.g., a target or desired I3). The current control module can set an initial voltage at the power supply, which generates a current, I1. The measured value of I1 is sent to a first current meter, which sends a corresponding signal to the controller. As the current travels from the emitter to the counter-electrode, there is parasitic current loss, I2. The actual value of the current that reaches the counter-electrode is given by I3. A current meter is used to monitor I3 and send a corresponding signal to the current control module. The current control module receives the signals which correspond to the values of I3 and I1, and generates and sends an adjustment control signal to the power supply, which alters the voltage potential between the emitter and the counter-electrode, thereby altering the current from the emitter, I1. In some implementations, the duty cycle of the control signal and/or output of the power supply can be varied in order to vary current.

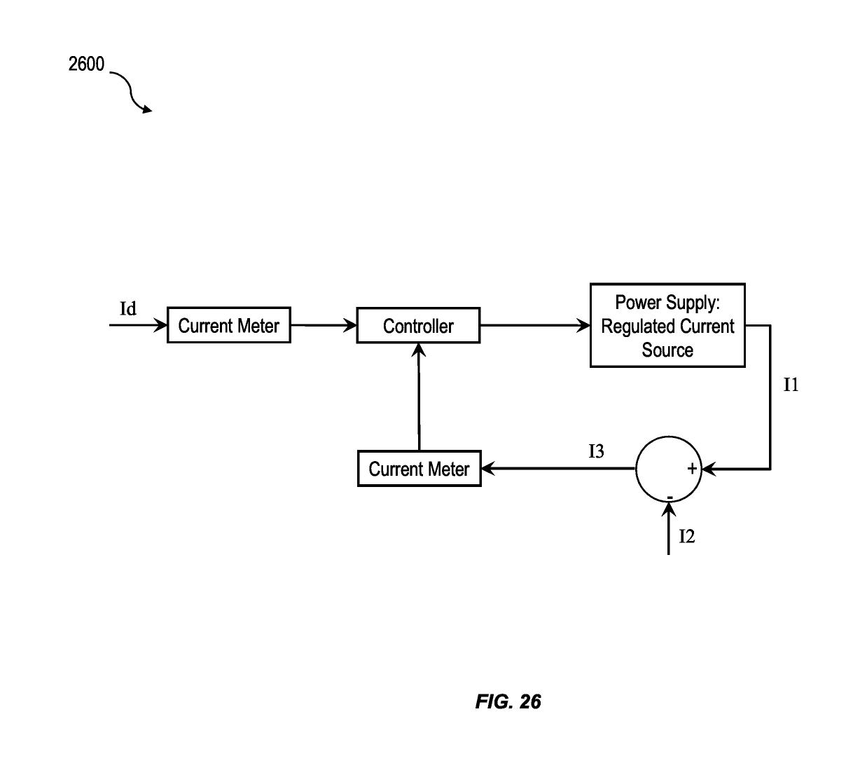

FIG. 26 is a system block diagram illustrating another example implementation of a current control module 2600 (e.g., as implemented by a controller). The current control module receives a desired counter electrode current value (Id). The desired counter electrode current value may be based, for example, on an intended application. The current control module sets an initial nominal voltage at the power supply, which generates a current, I1. As the current travels from the emitter to the counter-electrode, there is parasitic current loss, I2. The actual value of the current that reaches the counter-electrode is given by I3. A current meter is used to monitor I3 and send a corresponding signal to the controller. The controller receives the signals, which correspond to the values of I3 and the desired current value, Id, and generates and sends an adjustment control signal to the power supply which alters the voltage potential between the emitter and the counter-electrode, thereby altering the current from the emitter, I1.

In some implementations, the current control module can control the emitter current (I3) by pulse width modulation of a control signal of a switch that connects the power source and an emitter. For example, referring again to FIG. 1, an electrospray device can include individually controllable switches 120 enabling electrical excitation of emitters 125. The control signal to a given switch can be pulse width modulated to have a particular duty cycle. To adjust the emitter current (I3), the current control module can vary the duty cycle of the PWM control signal. For example, to increase the current the duty cycle of the control signal can also increase.

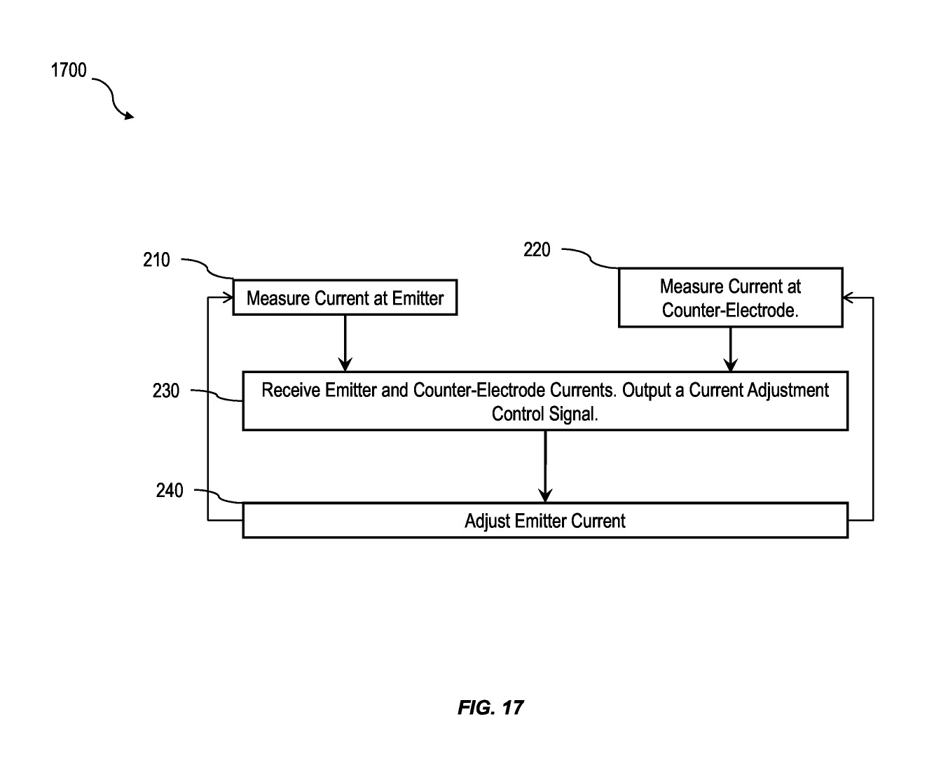

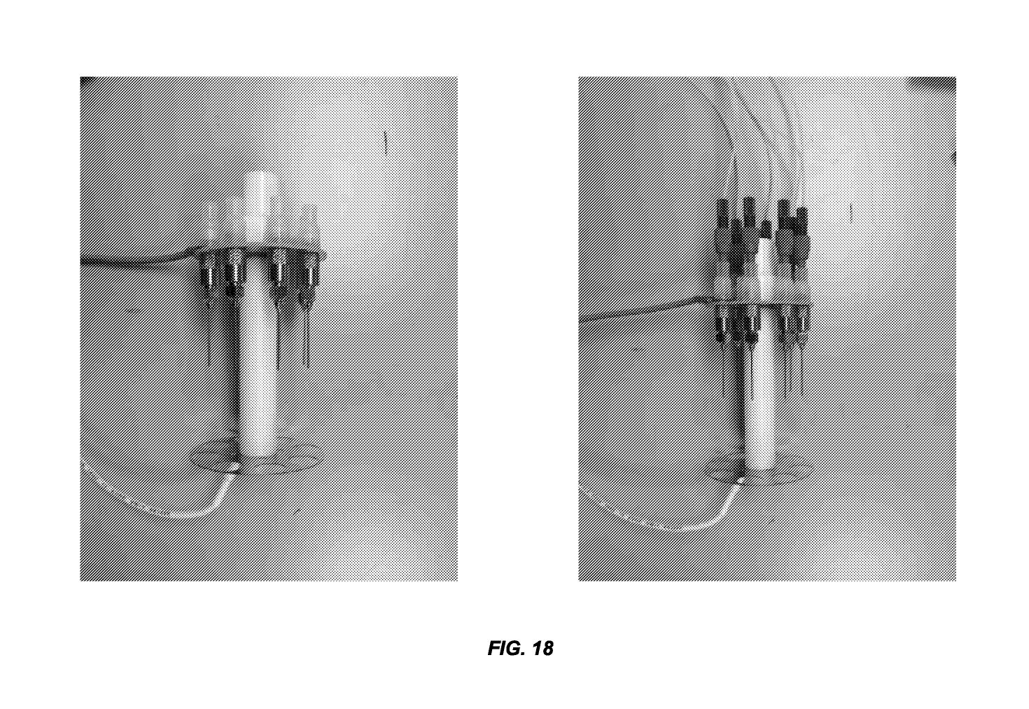

FIG. 17 is a process flow diagram illustrating a method 1700 for adapting an electrospray system (for example, the electrospray system illustrated in FIGS. 1 and 2) to compensate for variable humidity. At 210, the current at the emitter can be measured and at 220, the current at the counter electrode can be measured. At 230, a controller can receive the measured emitter current and counter-electrode currents and determine an adjustment or modification to the current source that will compensate for parasitic losses during the electrospraying process. At 240, the controller can send a signal to the current source to adjust the current levels in the system. This approach enables the system to operate independently of ambient conditions, such as humidity, by adjusting other components to compensate based on a current control signal. Further, by monitoring current at both the emitter and the counter-electrode, the accuracy of the control system is improved.

Some electrospraying systems include of a number of discrete subsystems connected via cabling and tubing allowing for individual interchangeability but in some cases display unideal behavior due to compatibility issues. Some implementations of the current subject matter integrate these subsystems into one assembly to mitigate for compatibility issues between fluidic, mechanical and electrical subcomponents.

Using the example implementation of the device requires no prior knowledge of microfluidics, high voltage circuitry design or electrospraying as operation will be through toggle on/off switches. Monitor and control of all system parameters can be automated and no user input can be required

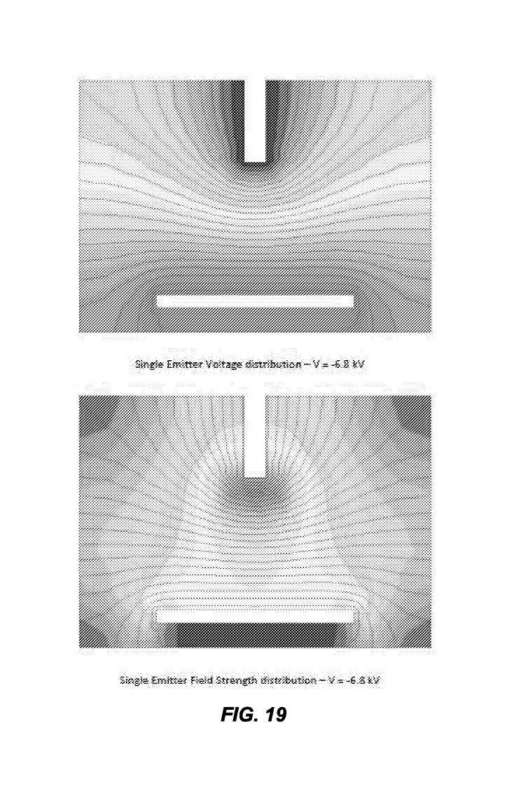

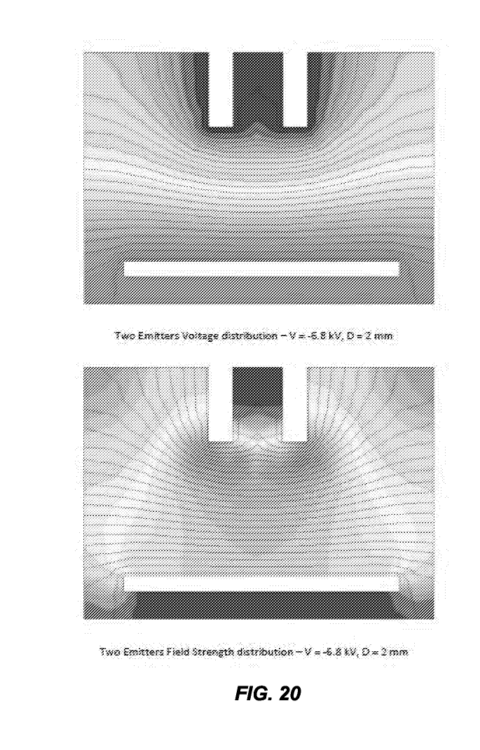

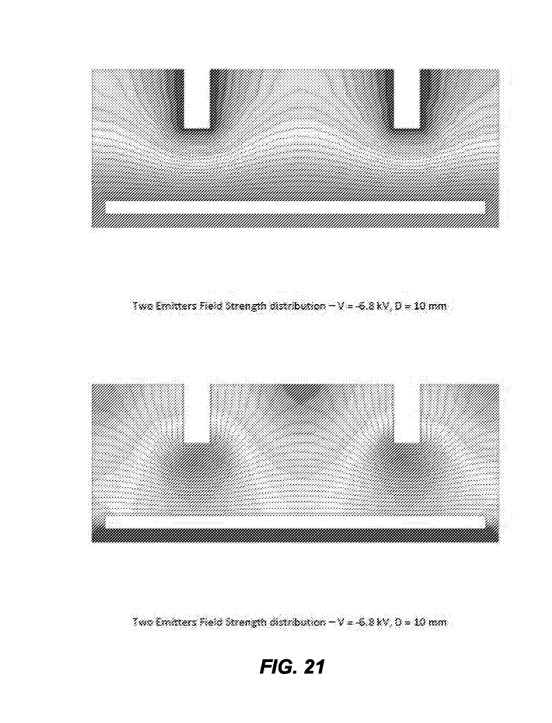

A repeating pattern of emitters and counter electrodes enables scale up of the system and increase in its throughput. For example, FIG. 18 illustrates an emitter array with eight emitters spaced in a circular arrangement (note that only 7 of the 8 emitters have tubing connected to provide solution in the shown image). Each emitter has a corresponding counter-electrode. However, emitters operating too closely in proximity interfere with one another's ability to create a proper Taylor cone needed for the electrospray process. FIG. 19-21 illustrate the voltage and field strength distribution for three scenarios. At FIG. 19, the voltage and field strength are illustrated for a single emitter and corresponding counter-electrode. The electrical potential difference between the emitter and counter-electrode is −6.8 kV. These conditions are ideal for establishment of the Taylor cone and performing electrospray in a EWNS application. FIG. 20 illustrates the voltage and field strength for two emitters operating simultaneously at a distance of 2 mm. As illustrated, the dual emitters create a field that inhibits Taylor cone formation. However, in the scenario of FIG. 20, if the emitters are alternatively strobed at 1 ms intervals, both emitters will establish cones and electrospray. FIG. 21 illustrates the voltage and field strength distributions for two emitters spaced 10 mm apart. Because of the distance between the emitters, there is limited interference and they can both create a Taylor cone while operating at the same time.

Thus, where emitters are proximal to one another, rapidly switching the electrical potential on and off using fast electrical switches (e.g., insulated-gate bipolar transistor (IGBT)) or high voltage metal oxide silicon field effect transistors (MOSFET) and a microprocessor sequence can mitigate or eliminate interference from neighboring emitters. And strobing allows denser placement of emitters on arrays. In operation, the microfluidic flow of solution to each emitter can be constant (e.g., uninterrupted) but the energizing of the emitter and/or counter-electrode to create a high potential difference is strobed. Thus, solution is continually provided.

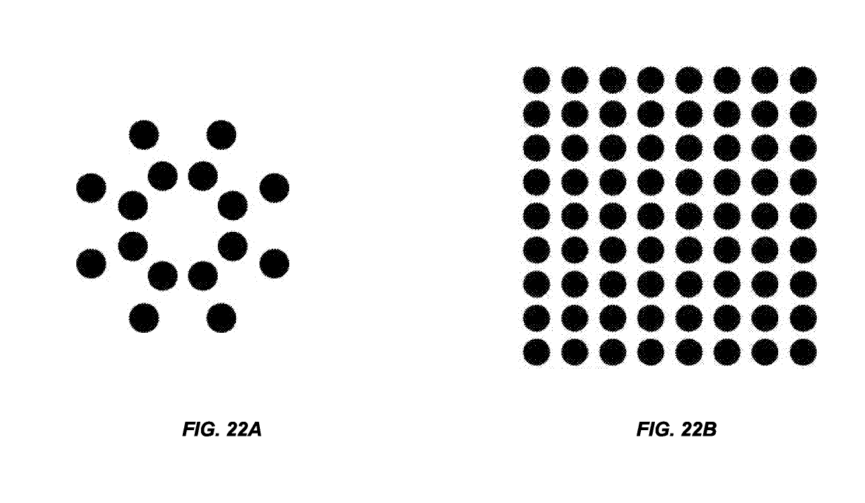

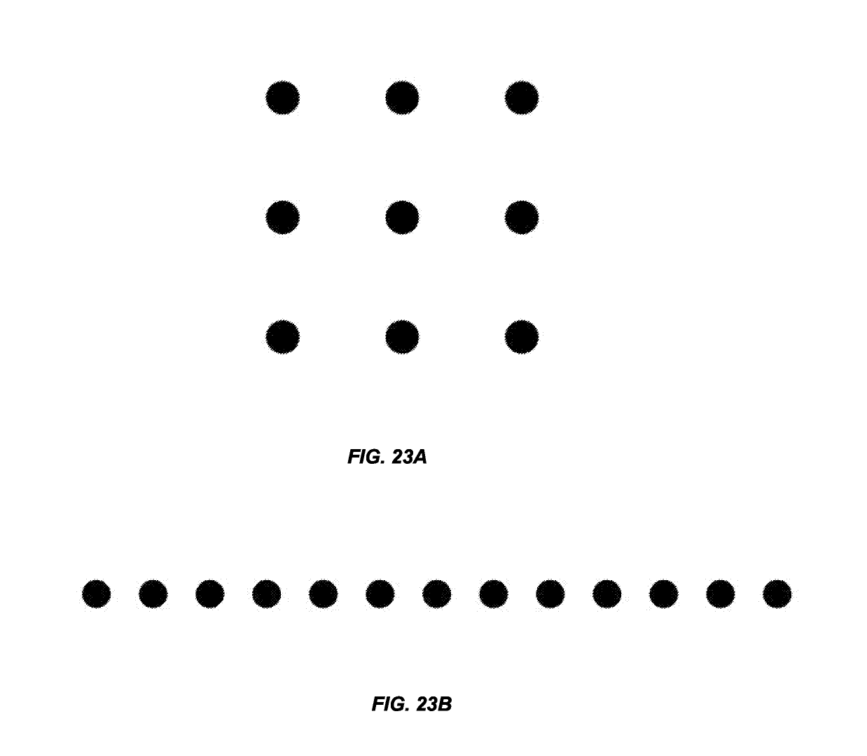

FIGS. 22A-B and 23A-B illustrate four example emitter array arrangements. Each arrangement can have associated with it a predetermined sequence of applied high electrical potential to each emitter so as to minimize emitter interference and allow for high-throughput electrospraying. Only one emitter in an array, or a set of emitters that will not create interfering fields, are fired simultaneously. For example, at a given instant none, one, some or all emitters may be energized. Energization intervals of 1 mS on 10 mS off can sustain an electrospray.

In one implementation, it has been found that emitters in an array interfere when they are spaced 2 mm but do not interfere when they are 10 mm apart. Thus, any two emitters in the array within 10 mm of one another should not be simultaneously connected to the high-voltage power supply. However, two emitters in the array that are spaced greater than 10 mm may be simultaneously connected to the high-voltage power supply. In addition, in the implementation, a rest period of up to 9 mS between consecutive firing of emitters at 2 mm spacing is attainable while maintaining electrospray from both. In some implementations, some rest period or guard interval can be desirable although in other implementations, no rest or guard period may be necessary. In some implementation, with sufficient applied voltage, an electrospray process can be sustained for a pulse width of 1 mS (Fswitch=1000 Hz=>T=0.1 ms; D=50%=>Energised time=0.5 mS).

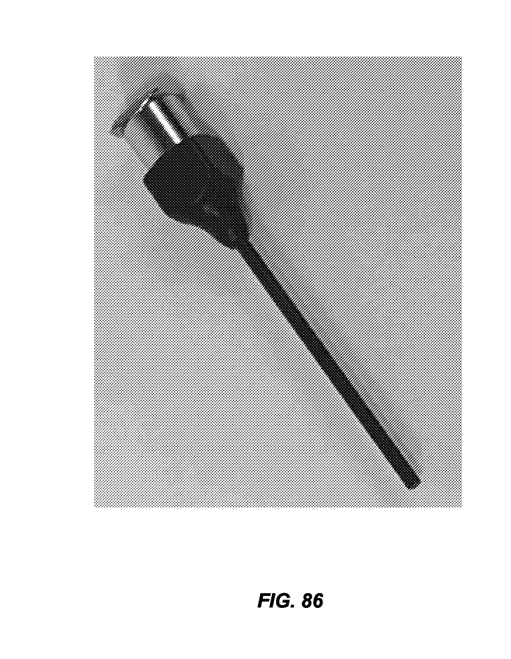

Another approach to reduce the interference effect of neighboring emitters in emitter arrays is to coat the emitters with Fluorinated ethylene propylene (FEP) or another suitable dielectric. For example, FIG. 86 illustrates an emitter coated with FEP.

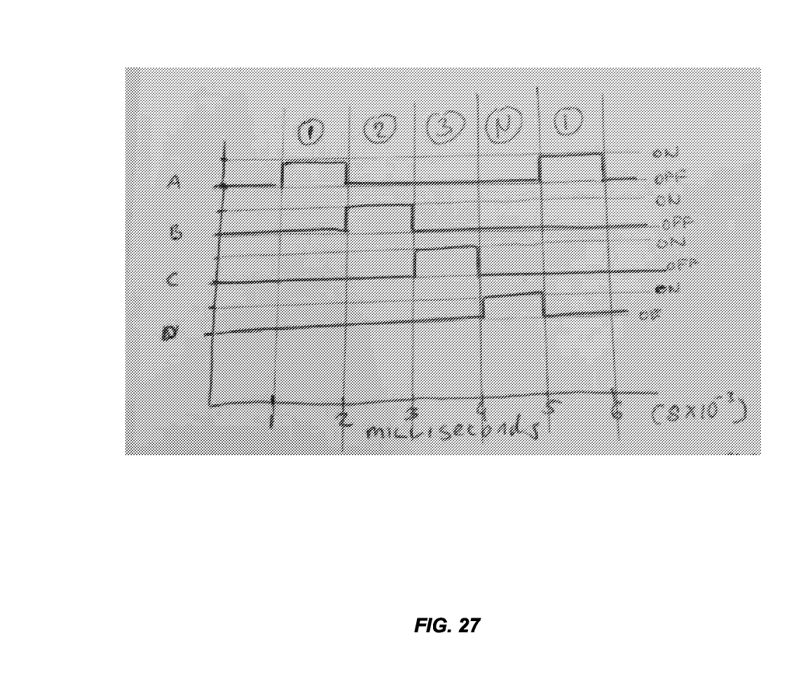

FIG. 27 is an emitter excitation timing diagram illustrating example control signals to selectively activate emitters in an array of N emitters. In the illustration, there are 4 neighboring emitters (e.g., N=4). The emitters are neighboring such that if they were simultaneously activated (e.g., connected to the high-voltage power supply) they would interfere with one another. Assuming each emitter is to be energized for 1 ms at a time, each emitter control signal (denoted by A, B, C, and D) includes a square wave that is logically high for 1 ms then logically low for 3 ms. When a given control signal is logically high, the corresponding emitter can be connected to the power supply. Thus, the control signals (A, B, C, and D) will cycle through activating emitters one at a time such that each emitter is activated while no two emitters are activated at the same time. While FIG. 27 illustrates the control signals A, B, C, and D as non-overlapping square waves that are arranged in sequence such that one emitter control signal is almost always high, guard intervals can be introduced so as to reduce any unintended emitter interference in the electrospray process.

In some implementations, an appropriate control frequency to accommodate a given sequence of energizing emitters in an array can be selected. Signals with long de-energized times (e.g., short duty cycles) can be overcome by increasing the applied voltage. Alternatively or in addition, a smaller switching frequency can be selected. Longer signal periods allow for lower applied voltages.

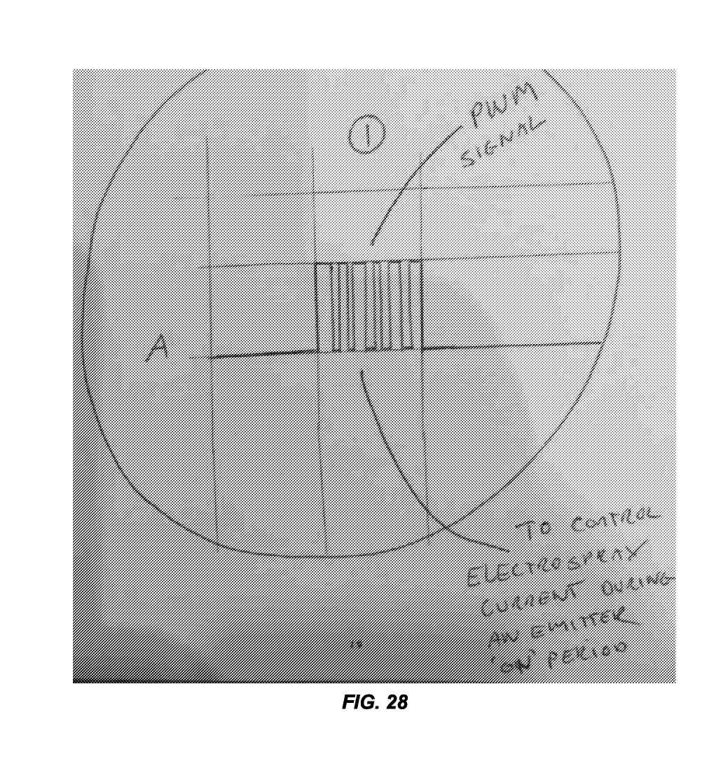

FIG. 28 is an emitter excitation timing diagram illustrating pulse width modulation of a single control signal pulse as illustrated in FIG. 27. For example, for a given time period in which an emitter activation control signal is logically high, the control signal can be pulse width modulated (PWM) to reduce overall current flow through the emitter. The control signal may, for example, have a 75% duty cycle. Such a duty cycle reduces the current flow as compared to a non PWM signal. PWM is an approach for modifying emitter current.

Additional modulation schemes are possible for manipulating emitter current. For example, the control signals can be sinusoidal to affect emitter current similar to PWM. In addition, the control signal amplitude can operate the emitter switches 120 within their linear operating region such that the emitter switches 120 act as variable resistors, which affects emitter current flow.

In one application the current subject matter can generate engineered water nano structures (EWNS) that comprise of reactive oxygen species (ROS) for inactivating at least one of viruses, bacteria, bacterial spores and fungi. In another application the current subject matter may be used to encapsulate living cells.

In another application, an electrospraying system with a consistent output may be used to encapsulate living cells or chemical compounds such as therapeutic or diagnostic agents.

Compounds described herein are purified. The polypeptides and other compositions of the invention are purified. For example, a polypeptide is preferably obtained by expression of a recombinant nucleic acid encoding the polypeptide or by chemically synthesizing the protein. A polypeptide or protein is substantially pure when it is separated from those contaminants which accompany it in its natural state (proteins and other naturally-occurring organic molecules). Typically, the polypeptide is substantially pure when it constitutes at least 60%, by weight, of the protein in the preparation. Preferably, the protein in the preparation is at least 75%, more preferably at least 90%, and most preferably at least 99%, by weight, AAH. Purity is measured by any appropriate method, e.g., column chromatography, polyacrylamide gel electrophoresis, or HPLC analysis. Accordingly, substantially pure polypeptides include recombinant polypeptides derived from a eucaryote but produced in E. coli or another procaryote, or in a eucaryote other than that from which the polypeptide was originally derived. Chemical compounds are purified from a natural source or synthesized.

As used herein, an “isolated” or “purified” compound is substantially free of other compounds or compositions with which it occurs in nature. Purified compounds, e.g., nucleotides and polypeptides are also free of cellular material or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified nucleotide or polypeptides is one that is at least 90%, 91%, 92%, 93%, 94%, 95%, 96%, 97%, 98%, 99%, or 100% (w/w) of the desired oligosaccharide by weight. Purity is measured by any appropriate standard method, for example, by column chromatography, thin layer chromatography, or high-performance liquid chromatography (HPLC) analysis. “Purified” also defines a degree of sterility that is safe for administration to a human subject, e.g., lacking infectious or toxic agents.

Cells, e.g., immune cells such as macrophages, B cells, T cells, used in the methods are purified or isolated. With regard to cells, the term “isolated” means that the cell is substantially free of other cell types or cellular material with which it naturally occurs. For example, a sample of cells of a particular tissue type or phenotype is “substantially pure” when it is at least 60% of the cell population. Preferably, the preparation is at least 75%, more preferably at least 90%, and most preferably at least 99% or 100%, of the cell population. Purity is measured by any appropriate standard method, for example, by fluorescence-activated cell sorting (FACS).

Small molecules are organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecule inhibitors can be synthetically engineered to consist of a fragment, or small portion, or a longer amino acid chain to fill a binding pocket of an enzyme. Typically small molecules are less than one kilodalton. In some examples, a small molecule has a molecular mass of less than 500 daltons.

The following lists provide example polymers, cells of interest, and applications for encapsulating cells.

Example polymers (purified from natural sources):
Alginate
Chitosan
Gelatin
Collagen
Cellulose
Chitin

Example Synthetic Polymers (Made Using Chemcical Process by Human Agency as Opposed to Derived or Purified from a Natural Source):
Aliphatic polyesters PLA, PGA and Poly(D,L-lactide-co-glycolide) (PLGA)
Poly vinyl pyrrolidone (PVP)
Poly vinyl alcohol (PVA)
Poly caprolactone (PCL)
Polyurethane (PU)
Polyamides (nylon)
Polyphosphazines
Polyepoxides
PEGDA
PVA
Polyacrylates
Poly(ethylene-co-vinyl alcohol)
Polyglycolic acid and chitin
Polysebacate
Poly(DTE carbonate)

Example Cells of Interest:
Pancreatic Islet cells, e.g., pancreatic beta cells
Human mesenchymal stem cells (MSCs)
T-cells (T lymphocytes)
NK-cells (natural killer cells)
CAR-T (Chimeric Antigen Receptor T cells)
CAR-NK (Chimeric Antigen Rceptor natural killer cells)
Pluripotent stem cells beta cells
Endothelial cells
HSC (hematopoetic stem cells)
DCs (Dendritic Cells)
iPSC (induced Pluripotent Stem Cells).
Oligodendrocytes
CHO (Chinese Hamster Ovary) cells
Human fibroblasts
Human Erthrocytes
Hybridoma cells
Leukocytes
Macrophages
human aortic smooth muscle cells and human dermal cells
aortic smooth muscle cells
human epidermal fibroblasts
human mammary carcinoma
human cervical carcinoma
Human Umbilical Vein Endothelial Cells
Human dermal fibroblasts
Human mesenchymal stem cells
human coronary artery endothelial cells
Dissociated DRG (dorsal root ganglia), Schwann cells, olfactory ensheathing cells, fibroblasts
human oral keratinocytes, human epidermal keratinocytes, human gingival fibroblasts
Modified/engineered versions of any of these cells e.g. MSC loaded with a cargo or gene edited
Glial cells or other cells of the central nervous system or peripheral nervous system

Example Applications:
Diabetes
Drug delivery
Cell and Gene therapy
Immunooncology
Production of myelin for multiple sclerosis patients (or similar neuro conditions)
Angiogenesis
general tissue engineering
vascular tissue engineering
neural tissue engineering
skin tissue engineering

In another application, an electrospraying system with a consistent output may be used to encapsulate drugs e.g., small molecule drugs, or polypeptide drugs, e.g., antibodies. The following lists provide example polymers, drugs of interest, and applications for encapsulating drugs.

Example Polymers:
Alginate
Polycaprolactone
Poly(DL-lacide-co-glycolide) (PLGA)
Stearic acid & Ethyl cellulose
Polyvinylpyrollidone and tristearin
Chitosan
PCL and PEG
Dextran
Poly(E-caprolactone-co-ethyl ethylene phosphate); PCLEEP
PLLA/PEO
PEG-PLLA
PVA/PVAc
PCL/PGC-C18

Example Drugs/Therapeutic/Diagnostic Compounds and/or Compositions
Budesonide
Celecoxib
Carbanazepine
Tamoxifen
Naproxen
Ampicillin
Dexamethasone
BSA
BSA and lysozyme
nerve growth factor and BSA
mefoxin, cefoxin sodium
Azidothmidine (AZT), acyclovir (ACV), maraviroc (MVC)
Bis-chloroethylnitrosourea
Ciproflaxcin
Paclitaxel
SN-38

Example Applications:
Asthma
Anti-inflammatory
Anti-convulsant
Breast cancer treatment
antibiotic
enhanced bone formation
drug delivery system
antiretroviral

In another application, an electrospraying system with a consistent output may be used to form microspheres of alginate material in the size region 1 microns to 2500 microns. When the example electrospray device was operated in a cone jet mode, particles were 1 μm in size on average whereas when operated in dripping mode, particles tend to be larger.

In the case of EWNS the solution is deionised or distilled or tap water. In the case of an encapsulate or other solution mixing and sampling the composition close to the emitter using microfluidic approach is optimal.

In some implementations, cells accumulate in tubing providing solution to the emitters. This problem can be addressed by eliminating tubing by having the reservoir in direct fluidic contact with the emitter.

Alginate can have toxic effect on cells when they are mixed. In some implementations, cells and alginate are mixed close to the emitter needle thereby increasing the viability of cells.

Depending on composition, some algination of cells can lead to macro size beads or droplets. The size of the bead can be optically detected via software and used as a process input variable. For example, the electrospraying system can include an image acquisition device (e.g., camera) and images of droplets formed at the output of the emitter can be acquired using the image acquisition device. Machine vision algorithms can process the images to determine droplet size.

Machine vision algorithms can be used to detect and determine size morphology and contents of encapsulate droplets. In example implementations, these algorithms can be highly efficient and synthesized in a manner that suits deployment on a field programmable gate array (FPGA) on the back of the image acquisition device (e.g., vision sensor). Utilizing an FPGA can be advantageous because transferring information over a standard interface (USB, I2C and the like) may be too slow for fast droplet detection.

In some implementations, image processing to detect droplets occurs over a number of steps. In some implementations, algorithms such as Sobel operator, Gaussian blur and convex hull calculation can be used to detect and characterize droplets.

In some implementations, a prediction algorithm can reduce the vision region of interest (ROI) to a region where it is known the droplet will be falling under the influence of gravitational or electric field. This can make the algorithm faster and more reliable at droplet detection. This approach does not double-count droplets and enables increased rate of droplet production and detection to a range of 100 to 1000 droplets per second while still having robust detection, individualization and characterization of droplets.

Droplet sizing can be determined with reference to a feature within the vision field of known dimensions. Characterization can refer to size morphology and contents of the droplet where to drop may contain cells or other materials. In some implementations, the vision algorithm can count, record, control a droplet discriminator circuit (e.g., as depicted in FIG. 88).

In some implementations, the machine vision algorithm can inform the parameters of the droplet producing subsystem implementing vision based feedback and control of size morphology and droplet rate.

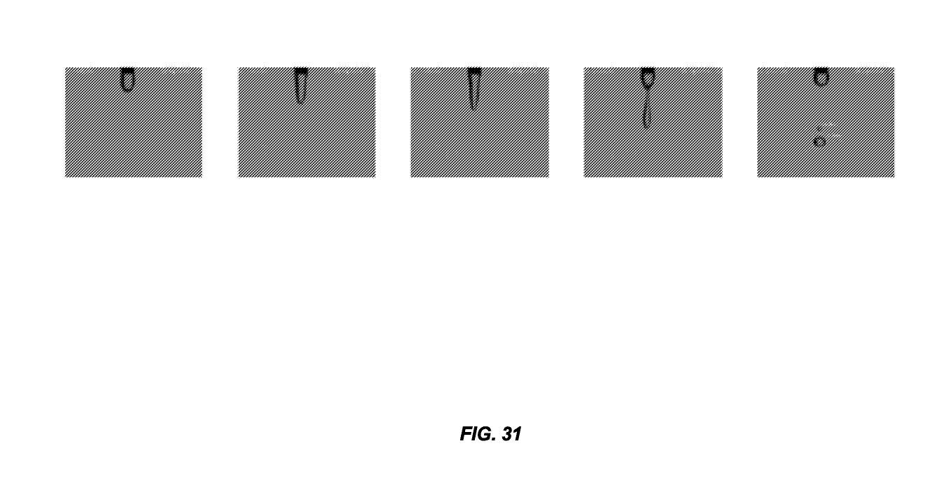

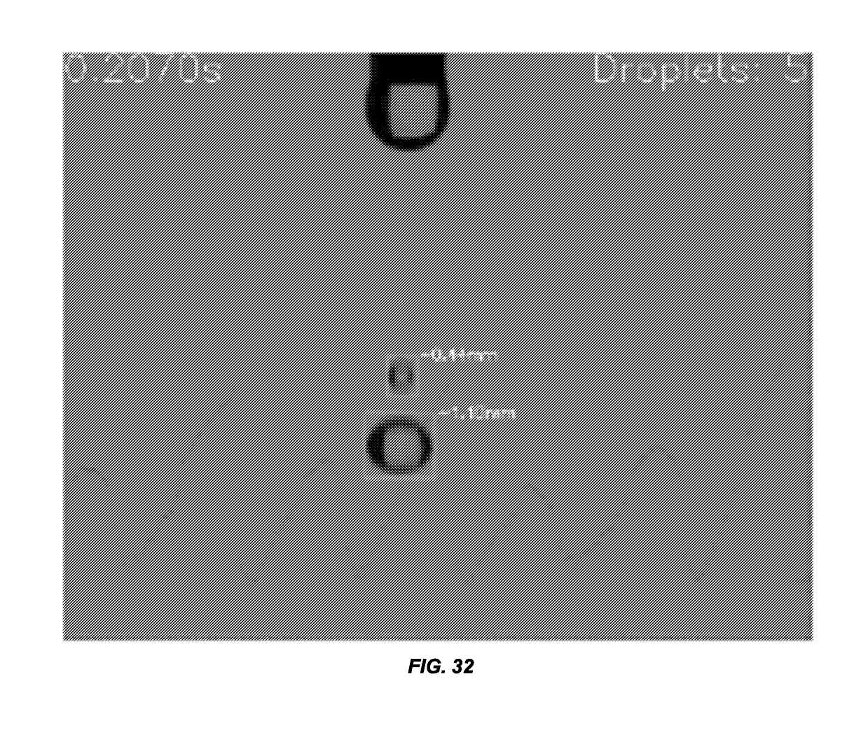

Images of an example particle sizing and tracking algorithm are illustrated in FIGS. 31 and 32. As illustrated, particles are detected when they break away from the Taylor cone and can be sized by machine vision algorithms. The machine vision algorithms can be implemented by the controller.

In another example implementation, the machine vision algorithms can be used as a means for particle sample control and selection. The electrospraying system can include a rejection element such as a as an electrode, charged plate, cross flow jets of air, mechanical trap doors or shut off valves located proximal to the emitter and/or counter-electrode. As particles (e.g., droplets) are created, their size can be detected and assessed. Particles outside of a predefined size range can be rejected by the system. In this example, the controller can detect and calculate the size of a particle and when it's outside the specified range (which may depend on a particular application, end use, and the like, of the output of the adaptive electrospray device), the electrostatically charged scrubber charged to the opposite polarity to the sprayed particle can be energized to alter the flight path of the particle before it reaches the counter-electrode (e.g., collector) and potentially spoiling the sample.

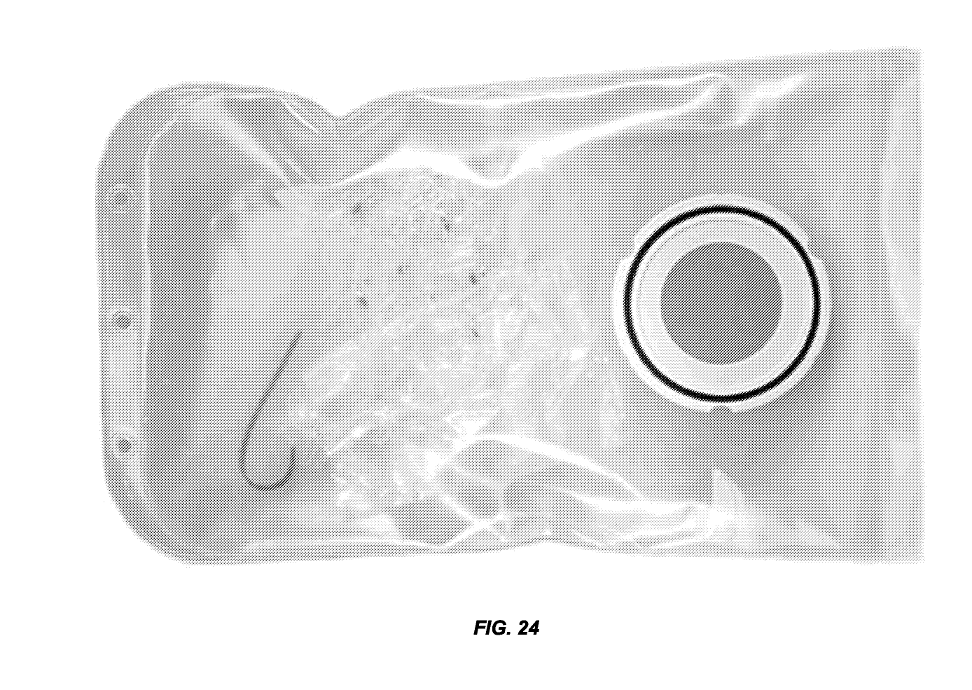

In some electrospraying implementations, cells are collected in a stainless steel dish containing cross-linking solution, normally calcium chloride. This arrangement is volumetrically self-limiting. Thus, in some implementations, encapsulated cells can be collected in a disposable bag. The collar of the bag can serve as the counter electrode or electrodes. For example, in some implementations, the counter-electrode can form an opening in a sterile bag. FIG. 24 is a photograph of a single use sterile bag for GMP cell processing and manufacture. The white circular fluidic filling connector can be replaced by a stainless or gold counter-electrode. In turn this can be addressed by one or an array of emitters. Thus, the current subject matter can be used for good manufacturing practices (GMP) manufacture. The bag is filled with the products of the electrospray process.

Other means of generating solution sprays include pneumatic generators such as nasal spray heads and other forms of nozzles. However, they generally do not produce monodisperse particles (particles with a homogeneous diameter distribution) and generally do not produce engineered water nano structures that comprise of reactive oxygen species.

The control, process, and design parameters that bring about a particular output for an electrospray device include spray solution chemical composition, solution flow rate, geometry and material properties of the emitter and collector, the high voltage potential difference between the emitter and counter electrode, the polarity of the voltage potential, the electrical field between the emitter and counter electrode, electrical fields from neighboring emitters or emitter arrays, oxidation or reduction of the emitter or counter electrode over time.

Electrospraying with the current subject matter can be used with different schemes for encapsulating cells or drugs. For example, co-axial electrospraying involves introducing cells and encapsulate at the point of spraying in co-axial streams. Another approach involves mixing cells and encapsulate in the same emitter or emitters at or close to the emitter. Another approach involves making spherical particles of a consistent size for downstream introduction of cells.

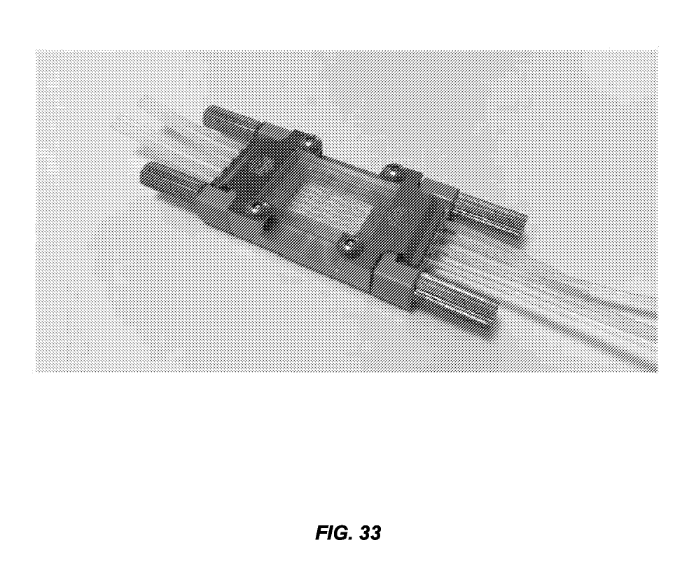

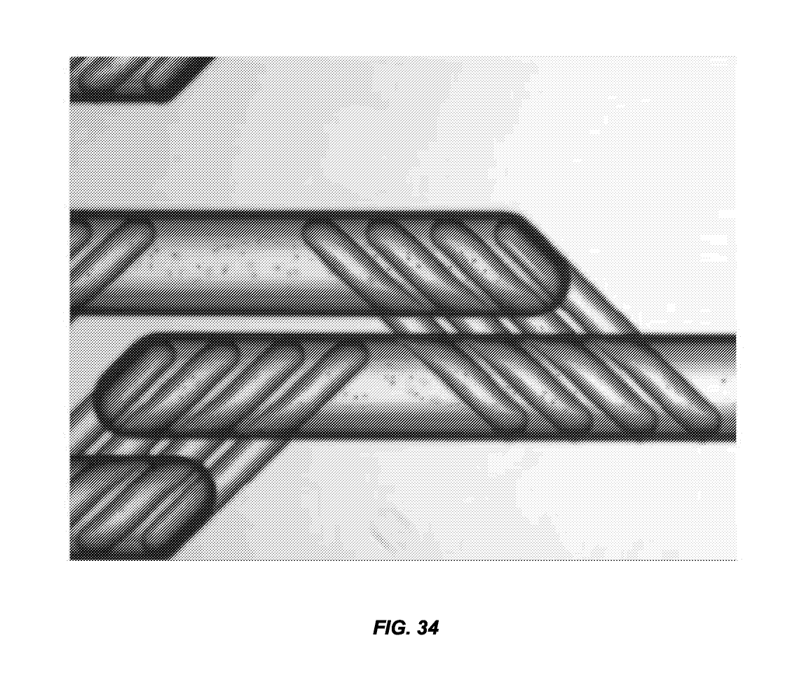

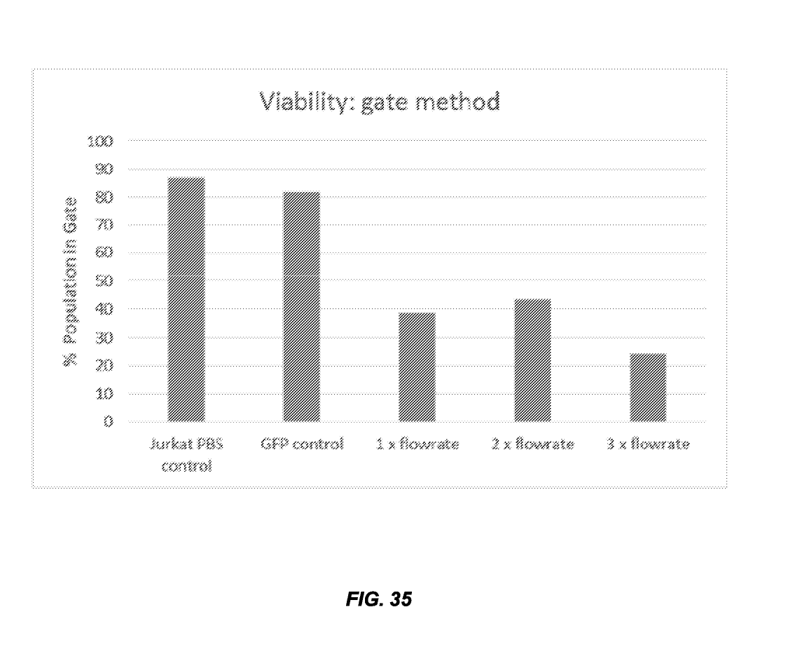

In some implementations, mixing of cells and encapsulate can be achieved via a microfluidic mixing chip such as Dolomite Microfluidics P/N 3200401 (Dolomite Bio of Royston, Hertfordshire, UK). FIGS. 33 and 34 illustrate the example microfluidic mixing chip. In FIG. 34, Jurkat cells in PBS are mixing within the example microfluidic mixer chip. FIG. 35 illustrates percentage of living Jurkat cells after mixing in the example Dolomite Microfluidic mixer chip.

Although a few variations have been described in detail above, other modifications or additions are possible. For example, while the electrospray device can adjust emitter current based on measured emitter and counter-electrode current, other parameters may be used in combination or in substitution for measuring current. For example, the electrospray device can include sensors to measure parameters directly such as humidity (e.g., as described in more detail below), temperature (e.g., as described in more detail below), voltage, solution composition, visual characteristics of solution (e.g., the current subject matter can be applied to a system as described in European Patent Application EP 3009828 filed Oct. 14, 2014, hereby incorporated by reference in its entirety; (FIGS. 3 and 19 illustrate how a droplet of unknown fluidic character can be measured visually at two points and its character determined), presence of bubbles in solution (e.g., via an optical sensor and detection of bubbles causes a stop and purge operation), and the like. In addition, current adjustment can be computed using numerical modeling, the Taylor-Melcher Leaky Dielectric Model (D. A. Saville, Electrohydrodynamics: The Taylor-Melcher Leaky Dielectric Model; Annual Review of Fluid Mechanics, Vol. 29: 27-64 (Volume publication date January 1997), DOI: 10.1146/annurev.fluid.29.1.27), and the like.

The current subject matter can be applied to improve biofabrication of compounds, materials, polymers and the like. For example, the current subject matter can improve biofabrication of alginate, alginate type material beads, and triazole containing analogues of alginate. The current subject matter can be used to tune or fine tune particle size, spherical dimension, monodispersity and the like of the electrospray output. Tuning of spherical dimensions of materials for delivery to tissue has been found to relate to the biocompatibility of a broad range of materials ranging through ceramics, metals, polymers, and the like (for example, as described in Vegas et. al, “Combinatorial hydrogel library enables identification of materials that mitigate the foreign body response in primates”; Nature Biotechnology, volume 34, number 4, March 2016, doi:10.1038/nbt.3462; and as described in Veiseh et al. “Size- and shape-dependent foreign body immune response to materials implanted in rodents and non-human primates”, Nature Materials, volume 4, June 2015, DOI: 10.1038/NMAT4290.

The subject matter described herein provides many technical advantages. For example, while some electrospray systems and devices are available such as SPRAYBASE® manufactured by Avectas, Ltd. Dublin, Ireland, they lack sufficiently consistent output and/or throughput for a large range of applications. In contrast, the current subject matter provides increased consistency and throughput in electrospray output. For example, the current subject matter can produce consistent alginate cells at scale and in a clinical process. The current subject matter provides tight integration of electrospray system, lack of tubes, use of microfluidics, and higher throughput enables high-throughput and consistent electrospray devices. Materials consistent with Good Manufacturing Process in the pharmaceutical industry.

The current subject matter can include a closed loop system, controlling for current distinct from open loop systems. The current subject matter may be immune from changes in humidity whilst electrospraying in atmospheric conditions distinct from systems which are affected by humidity changes. The current subject matter can produce more consistent particles, droplets, microspheres, colloids than the existing electrospray systems over longer periods. The current subject matter can produce consistent electrospray over protracted periods distinct from existing systems, which will drift away from starting operating parameters in a relatively short duration.

Some implementations of the current subject matter includes a device that highly integrated without interconnecting tubes unlike existing systems, which can be modular and interconnected by tubes. Some implementations of the current subject matter measures current in two places and uses this as an error and/or control signal.

Some implementations of the current subject matter enables pulse width modulation (PWM) control of individual emitters as a form of current control. Some implementations of the current subject matter include emitter arrays providing greater throughput capability unlike some existing electrospray systems, which utilize a single emitter.

Some implementations of the current subject matter can use a sterile bag as collection means. The current subject matter can be compatible with GMP cell manufacturing, encapsulation and engineering. The current subject matter can utilize FEP coated emitters to enable emitter arrays.

Some implementations of the current subject matter can produce microspheres in a range of sizes 1 um to 3 mm, consistently at scale <(2 mL/minute) and in a way consistent with GMP given choice of electrode and counter electrode materials (e.g., 306 stainless steel) and approved plastics. In some implementations, the current subject matter can produce microspheres in a range of 10 nm to 2500 micron, depending in part on the polymers of interest and the encapsulation application. For example, drug encapsulation may result in nanoparticles. Use of the current subject matter can control and/or eliminate electrospray commencement artifacts.

The current subject matter may be used, for example, to fabricate alginate hydrogel spheres and cell encapsulation as described in Vegas et. al “Long-term Glycemic Control using Polymer-Encapsulated Human Stem Cell-Derived Beta Cells in Immune-competent Mice, Nature Medicine, vol. 22, Number 3, March 2016, pp. 306-311; and online methods at doi:10.1038/nm.4030, the entire contents of which is hereby incorporated by reference in its entirety. In Vegas, fabrication of alginate hydrogel spheres and cell encapsulation was achieved. Prior to sphere fabrication, buffers were sterilized by autoclaving, and alginate solutions were sterilized by filtration through a 0.2-μm filter. Aseptic processing was implemented for fabrication by performing capsule formation in a type II class A2 biosafety cabinet to maintain sterility of manufactured microcapsules/spheres for subsequent implantation. An electrostatic droplet generator was set up in the biosafety cabinet as follows: an ES series 0-100-kV, 20-watt high-voltage power generator (Gamma ES series, Gamma High-Voltage Research, FL, USA) is connected to the top and bottom of a blunt-tipped needle (SAI Infusion Technologies, IL, USA). This needle is attached to a 5-ml Luer-lock syringe (BD, NJ, USA), which is clipped to a syringe pump (Pump 11 Pico Plus, Harvard Apparatus, MA, USA) that is oriented vertically. The syringe pump pumps alginate out into a glass dish containing a 20 mM barium 5% mannitol solution (Sigma-Aldrich, MO, USA). The settings of the PicoPlus syringe pump are 12.06 mm diameter and 0.2 ml/min flow rate. After the capsules are formed, they are then collected and then washed with HEPES buffer (NaCl 15.428 g, KCl 0.70 g, MgCl2.6H2O 0.488 g, 50 ml of HEPES (1 M) buffer solution (Gibco, Life Technologies, California, USA) in 2 liters of deionized water) four times. The alginate capsules are left overnight at 4° C. The capsules are then washed two times in 0.8% saline and kept at 4° C. until use.

To solubilize alginates, SLG20 (NovaMatrix, Sandvika, Norway, cat. #4202006) was dissolved at 1.4% weight to volume in 0.8% saline. TMTD alginate was initially dissolved at 5% weight to volume in 0.8% saline and then blended with 3% weight to volume SLG100 (also dissolved in 0.8% saline) at a volume ratio of 80% TMTD alginate to 20% SLG100.

0.5-mm spheres were generated with a 25G blunt needle, a voltage of 5 kV and a 200 μl/min flow rate. For formation of 1.5-mm spheres, an 18-gauge blunt-tipped needle (SAI Infusion Technologies) was used with a voltage of 5-7 kV. Immediately before encapsulation, the cultured SC-β clusters were centrifuged at 1,400 r.p.m. for 1 min and washed with calcium-free Krebs-Henseleit (KH) Buffer (4.7 mM KCl, 25 mM HEPES, 1.2 mM KH2PO4, 1.2 mM MgSO4×7H2O, 135 mM NaCl, pH=7.4, =290 mOsm). After washing, SC-β cells were centrifuged again and all of the supernatant was aspirated. The SC-β pellet was then resuspended in the SLG20 or TMTD alginate solutions (described above) at cluster densities of 1,000, 250, and 100 clusters per 0.5 ml alginate solution. Spheres were crosslinked using a BaCl2 gelling solution, and their sizes were controlled as described above. Immediately after cross-linking, the encapsulated SC-β clusters were washed four times with 50 ml of CMRLM medium and cultured overnight in a spinner flask at 37° C. before transplantation. Owing to an inevitable loss of SC-β clusters during the encapsulation process, the total number of encapsulated clusters were recounted after encapsulation.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an recited feature or element is also permissible.

Characterizing an Example Electrospray Device using a High-Voltage, High Frequency Control Signal

In an example implementation of an electrospray device, a high voltage (HV), high frequency pulsar module, which can alternate a HV emitter between an energized and de-energized state, was used to induce a steady electrospray process of common solutions. It is shown that the duty cycle of the HV signal can effectively control the electrospraying process at a fixed applied voltage. In the example, above switching frequencies of 1 Hz, a duty cycle of 50% is not sufficient to induce an electrospray of common solutions at the same applied voltage that is sufficient to induce an electrospray when supplied by a constantly energized power supply source. Increasing the duty cycle of a pulsed HV signal has the same effect on the electrospraying process of common solutions as increasing the applied voltage when supplied by an “always on” power supply module.

Equipment used for these experiments included Behlke high voltage pulsar module—FSWP 91-01 with direct liquid cooling (BEHLKE POWER ELECTRONICS, Billerica, Mass.), Spraybase CAT000047—20 kV, 1 bar power supply (AVECTAS, Dublin Ireland), Edgertronic high speed camera—2000 FPS (SANSTREAK CORP, San Jose Calif.), and a Point Grey Chameleon monochrome camera—15 FPS.

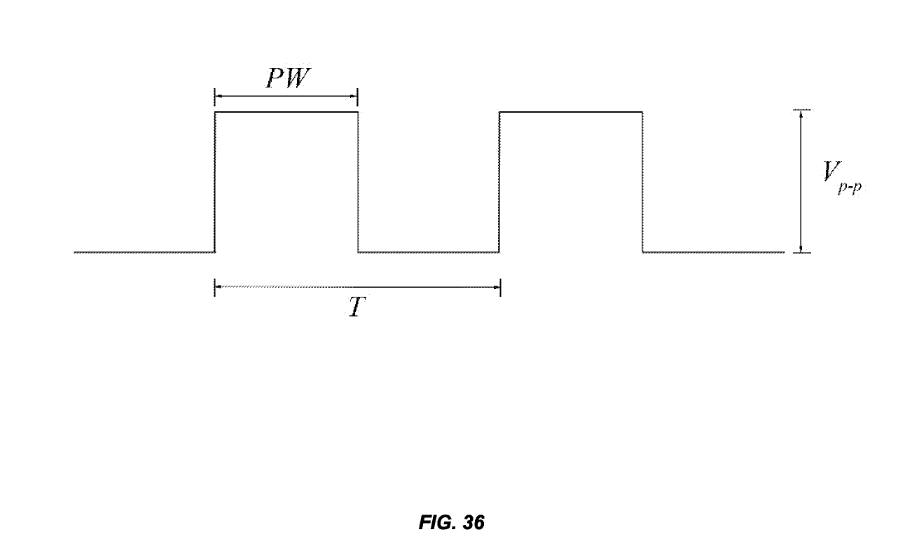

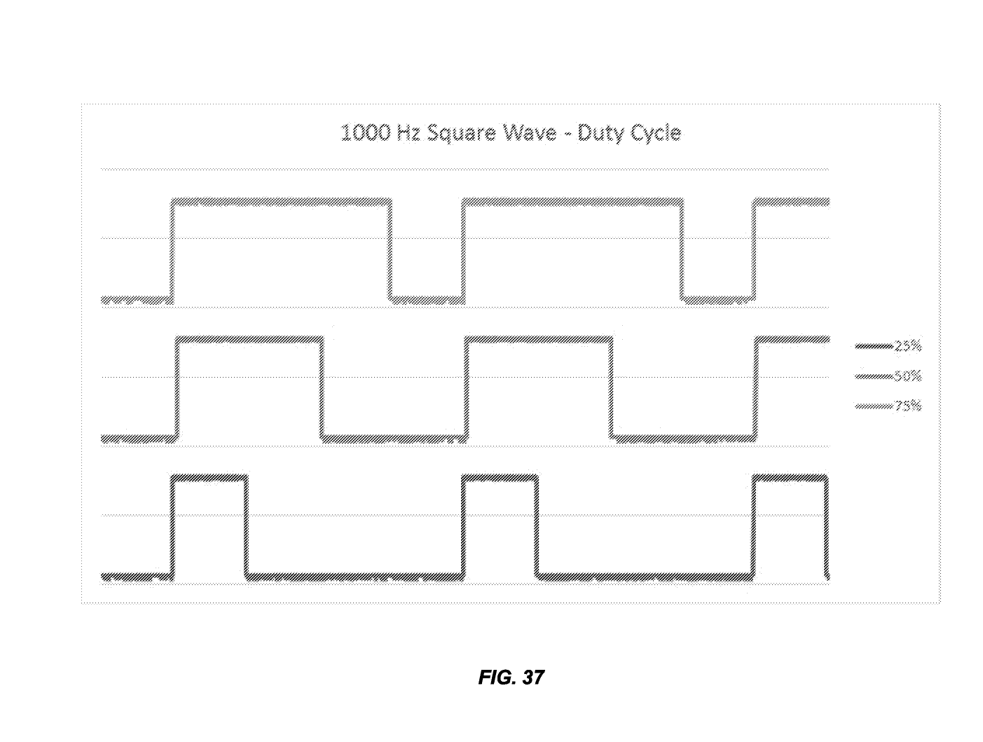

In these experiments a pulsed square wave signal was used to control the switching characteristics of the FSWP 91-01 HV pulsar module. The frequency and duty cycle of the square wave was altered to examine its effect on the electrospraying process of common solutions. The duty cycle (D) of a square wave signal can be defined as D=PW/T×100%. Where PW is the positive pulse active time and T is the period of a signal as defined in FIG. 36. Increasing the duty cycle has the effect of increasing the positive pulse active time as can be seen in FIG. 37, which compares various duty cycles for a 1000 Hz square wave.

The experimental parameters used for this work are as follows: Solution 1—70% EtOH; Solution 2—PBS 1×; KCl (Potassium chloride) 2.7 μM; KH2PO4 (Potassium phosphate monobasic) 1.5 μM; NaCl (sodium chloride) 138 μM; Na2HPO4 (Sodium phosphate dibasic) 8.1 μM; 20% v/v ethanol; P=250 mbar; Feed tube=Tube A (127 um ID, 1 m long); S=35 mm (emitter to collector separation); Emitter—30 G, 25 mm long; Fswitch—0 Hz, 1 Hz, 100 Hz, 1000 Hz; and Duty Cycle 10%-90%.

The experimental logic was as follows. Step 1—A control experiment was completed to determine the applied voltage required to induce a stable electrospray for solutions 1 and 2 when the emitter was constantly energized (F=0 Hz). Step 2—At these applied voltages, the switching function of the FSWP 91-01 pulsar was enabled with a square wave at frequencies of 1 Hz, 100 Hz and 1000 Hz. D was varied and images of the corresponding electrospray were recorded using a slow speed (15 frames per second) Point Grey Chameleon monochrome camera and a high speed (2000 frames per second) Edgertronic camera to determine the effect of duty cycle on the process. Step 3—For each frequency, solution and applied voltage, a minimum duty cycle at which no electrospray could be induced was determined. The applied voltage was then increased until a stable electrospray was induced and the effect of varying D at this new applied voltage was recorded.

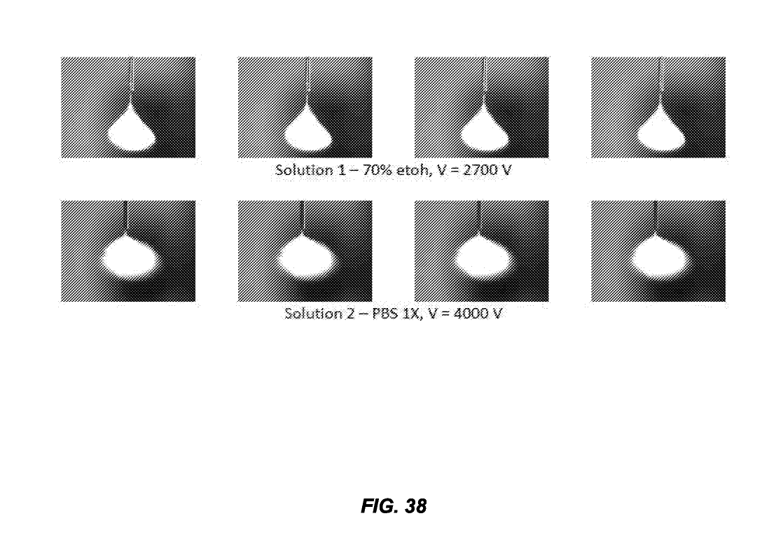

FIG. 38 shows a series of image captures of a stable electrospray process for solution 1 and solution 2 at their respective applied voltages when Fswitch=0 Hz over a duration of 266 mS. Note: These images were taken at a rate of 15 FPS and are consecutive images in the process.

What can be seen from the images is that over 266 mS of the spray, a steady Taylor cone and plume is induced for both solutions. Although not shown here, high speed recording of each spray at 2000 FPS shows that the process is pulsatile in nature. At 15 FPS, the oscillatory nature of the electrospray process cannot be resolved however at 2000 FPS, it is clear that in cone jet mode, the Taylor cone and plume show a pulsatile characteristic.

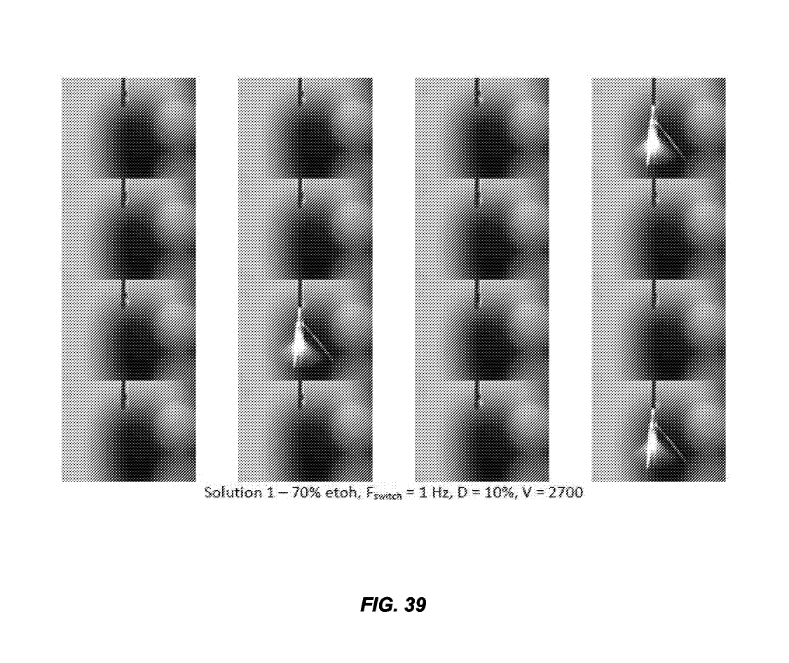

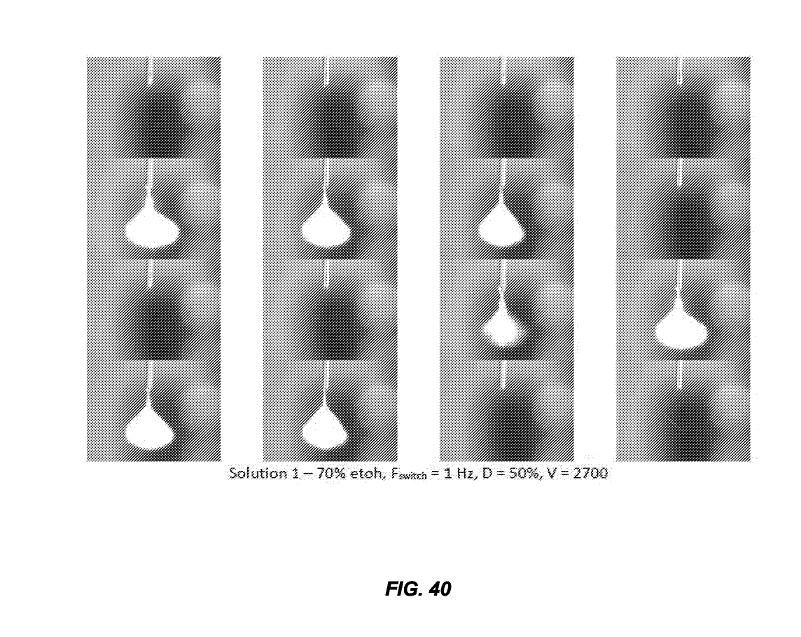

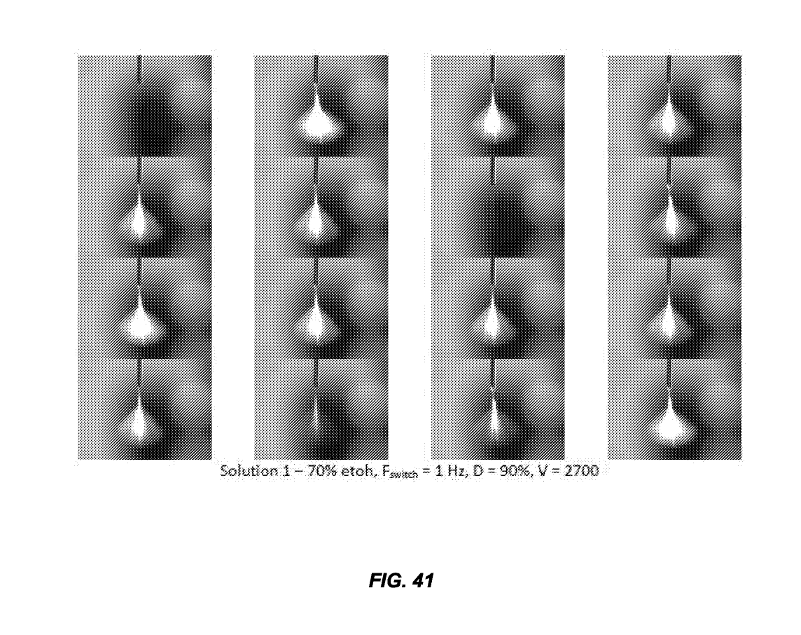

FIG. 39-41 show a series of image captures of the process for solution 1 at duty cycles of 10%, 50% and 90% respectively when the switching frequency is set to 1 Hz and the control voltage is 2700 V over a duration of 2 seconds. At D=10%, solution 1 shows a fast dripping characteristic with a period between drips of approximately 660 mS. The spray “on” time at D=10% is 100 mS. At D=50%, solution 1 shows a distinct pulsed spray characteristic where the spray “on” time is equivalent to the spray “off” time. The spray “on” time at D=50% is 500 mS. At D=90%, solution 1 shows a distinct fast pulsed spray characteristic where the spray “on” time is longer than the spray “off” time. The Spray “on” time at D=90% is 900 mS.

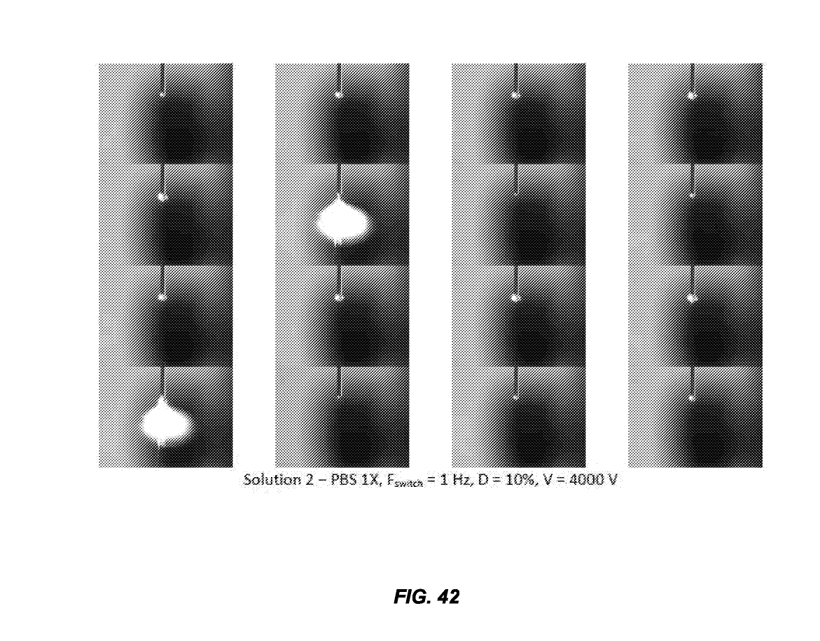

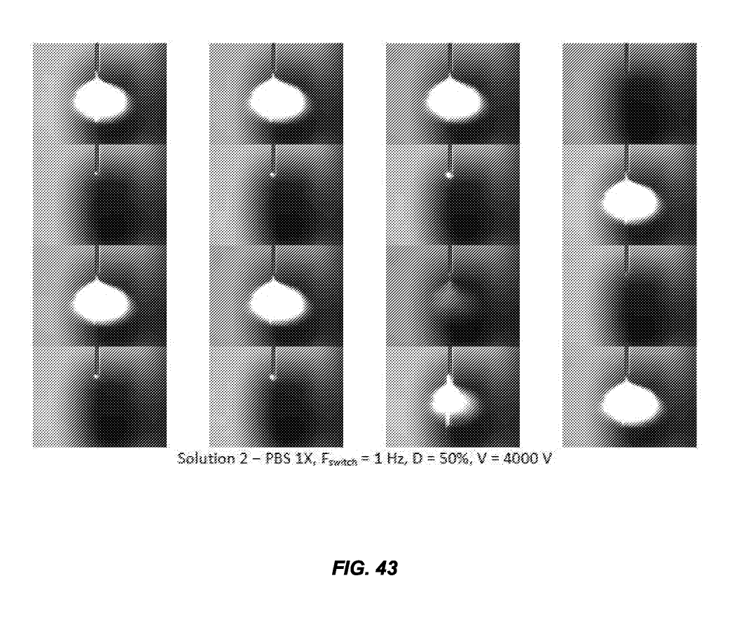

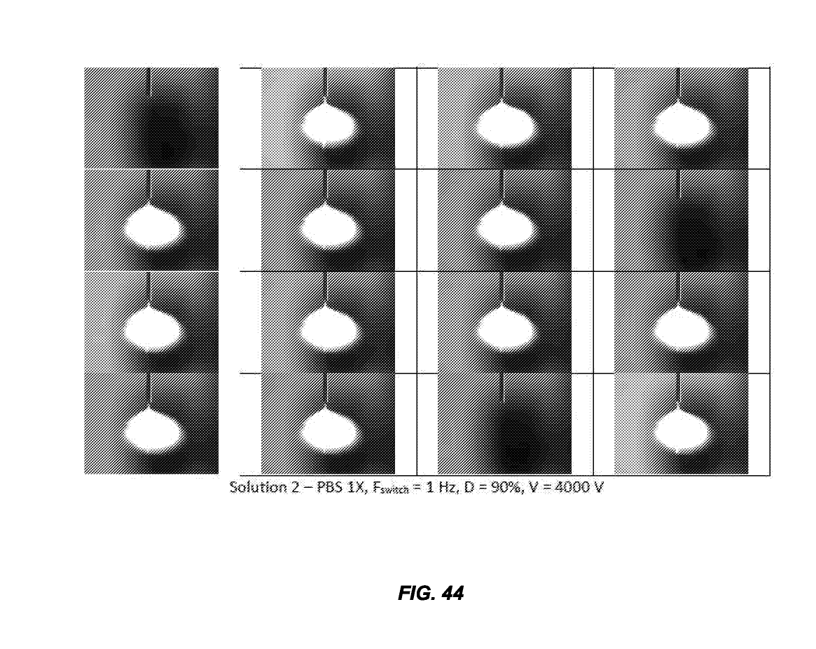

A similar behavior was observed for solution 2 and the results can be seen in FIGS. 42-44 for D=10%, 50% and 90% respectively. A higher applied voltage of 4000 V was required to induce a steady electrospray of solution 2 under control conditions (Fswitch=0 Hz) and the induced spray shows different characteristics compared to solution 1 however the effect of the duty cycle on the two solutions is equivalent.

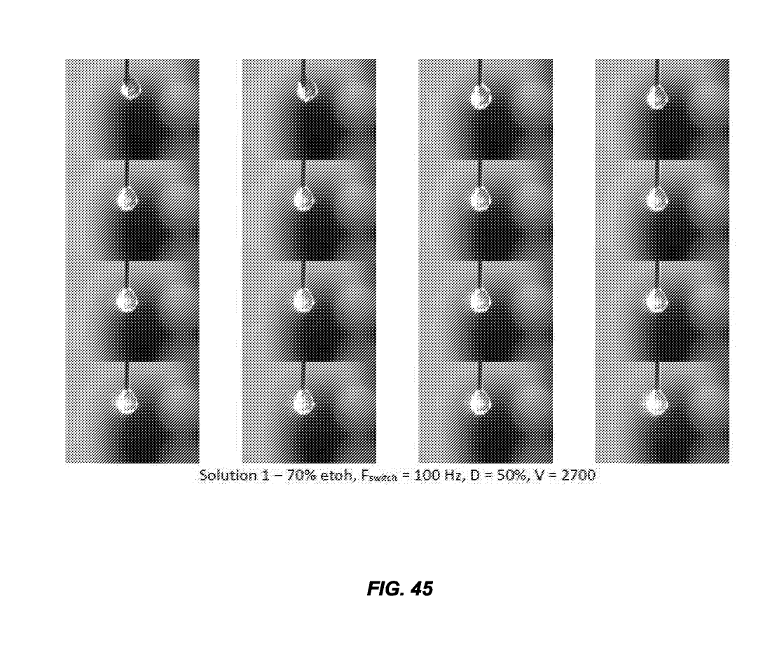

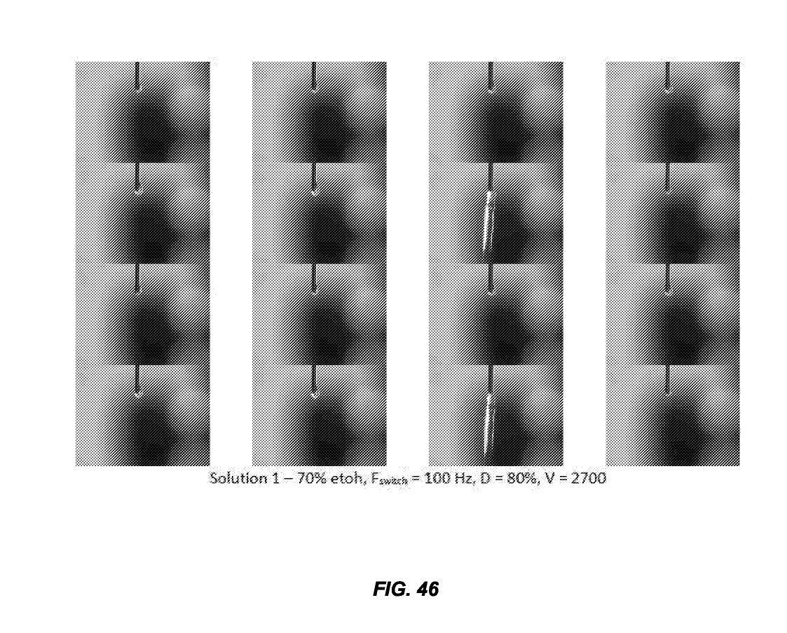

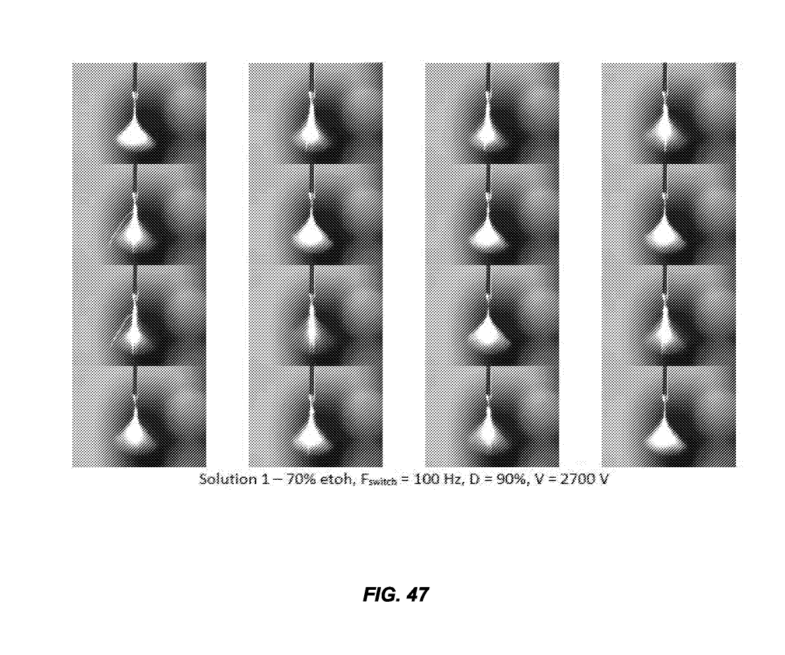

The frequency of the FSWP 91-01 HV pulsar was increased to 100 Hz and a similar experiment was repeated. FIGS. 45-47 show a series of image captures of the process for solution 1 at duty cycles of 50%, 80% and 90% respectively when the switching frequency is set to 100 Hz and the control voltage is 2700 V over a duration of 2 seconds. From FIG. 45 at D=50%, V=2700 V solution 1 experiences a pendant droplet. This was not observed at Fswitch=1 Hz. It appears that for Fswitch=100 Hz, the spray “on” time, which in this case was 5 mS is not sufficiently long to energize the system into a spray state. From FIG. 46, at D=80%, V=2700 V solution 1 experiences a fast dripping mode. The spray “on” time at D=80% is 8 mS which is similar to the Spray “on” time for D=10%, Fswitch=1 Hz. It is suspected that this is why their characteristic spray pattern is quite similar. From FIG. 47, at D=90%, V=2700 V solution 1 starts to experience an electrospray mode. The spray “on” time for this signal is 9 mS. The image captures in FIG. 47 show that the solution experiences a ramified cone jet mode and is not in the fully stable cone jet mode. Increasing the duty cycle of the HV pulser (that is increasing the Spray “on” time) for a fixed applied voltage appears to have the same effect as increasing the applied voltage when a solution is supplied by a constantly energized power supply in that solution transitions from a pendant droplet into an electrospraying mode as the voltage is increased. Hence, increasing the spray “on” time for a pulsing electrospray system appears to be equivalent to increasing the applied voltage.

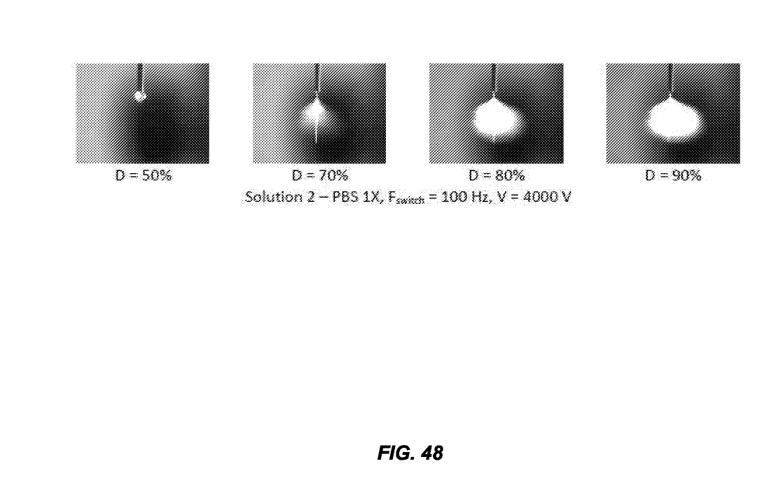

A similar behavior was observed for solution 2 when the Duty cycle was increased from 50% to 90%. FIG. 48 shows a single image capture of the process at various duty cycles. As can be seen the solution transitions from a pendant droplet into a full plume and that size and intensity of the plum increases with the duty cycle (i.e. with increasing spray “on” time). This is similar to what's observed when the emitter applied voltage is increased using a constantly energized power supply.

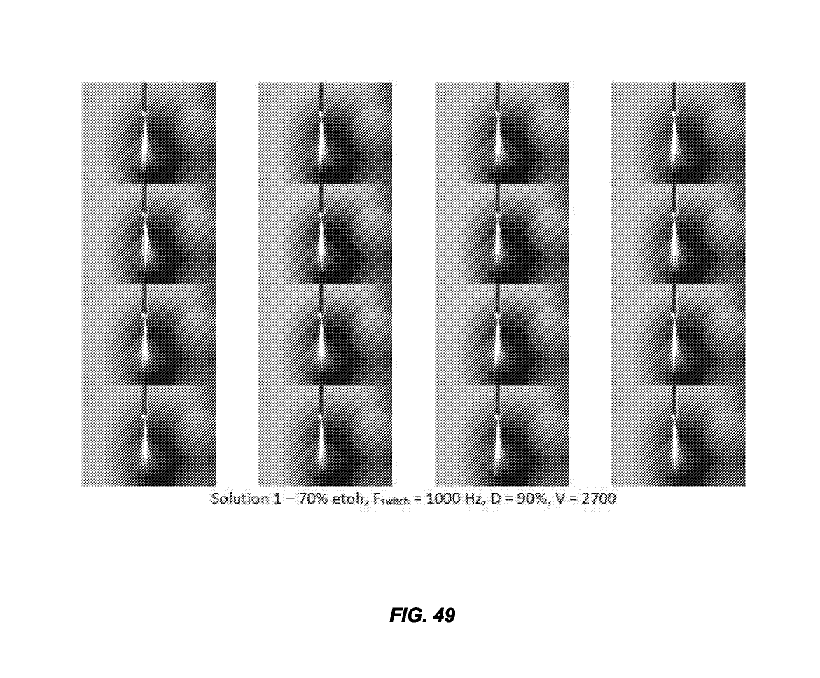

The frequency of the FSWP 91-01 HV pulser was increased to 1000 Hz and a similar experiment was repeated. Based on the results described above, it was decided to initially test a duty cycle of 90% to determine if a stable electrospray could be induced. FIG. 49 shows a series of image captures of the process for solution 1 at a duty cycle of 90% when the switching frequency is set to 1000 Hz and the control voltage is 2700 V over a duration of 2 seconds. At D=90%, V=2700V solution 1 experiences an overfed Taylor cone and chaotic spray. There is not sufficient energy in the system to induce a stable electrospray. It was decided to increase the applied voltage until a stable electrospray was induced.

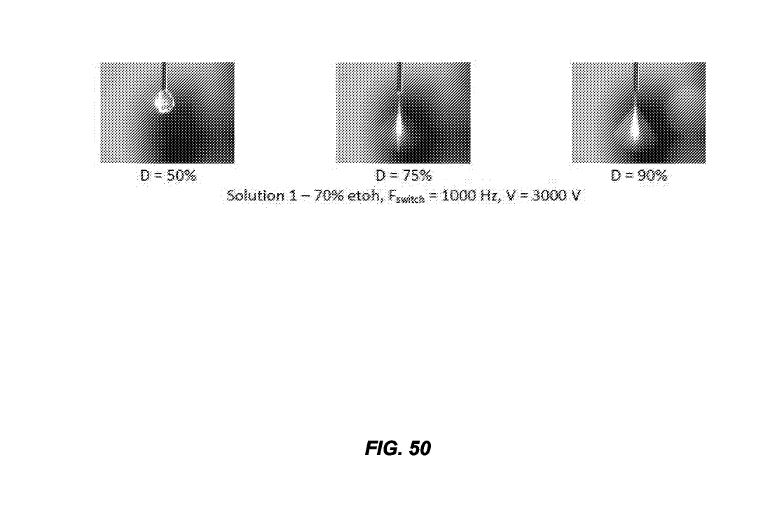

It was found that V=3000 V was sufficient to induce a stable electrospray for D=90%, Fswitch=1000 Hz. At this elevated voltage, the duty cycle was varied and its effect on the electrospraying process was observed. FIG. 50 shows a single image capture of the process for solution 1 at various duty cycles for V=3000 V. As can be seen the solution transitions from a pendant droplet into a full plume and the size and intensity of the plume increases with the duty cycle (i.e. with increasing spray “on” time). This is similar to that observed when the emitter is supplied by a constantly energized power supply and the applied voltage is increased.

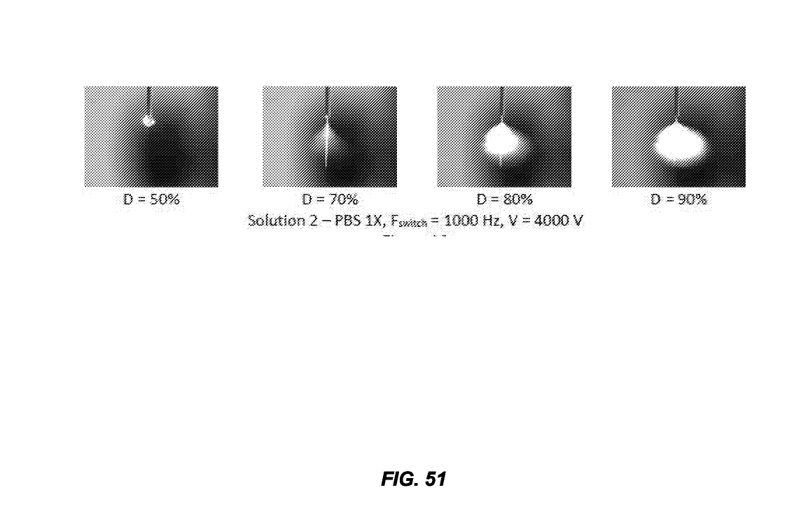

A similar effect was observed for solution 2 at Fswitch=1000 Hz and the results can be seen in FIG. 51.

The results show that increasing the duty cycle of HV signal can control the electrospraying process. It appears that for switching frequencies above 1 Hz, a duty cycle greater than 50% is required to induce a stable electrospray when the applied voltage is set to a value which induces a stable electrospray when supplied by a constantly energized power supply. Moreover, varying the duty cycle changes the mode of electrospraying experienced by the solution.

Further characterization exercises were conducted to examine if increasing the duty cycle will transition the electrospraying process into modes above the cone jet mode.

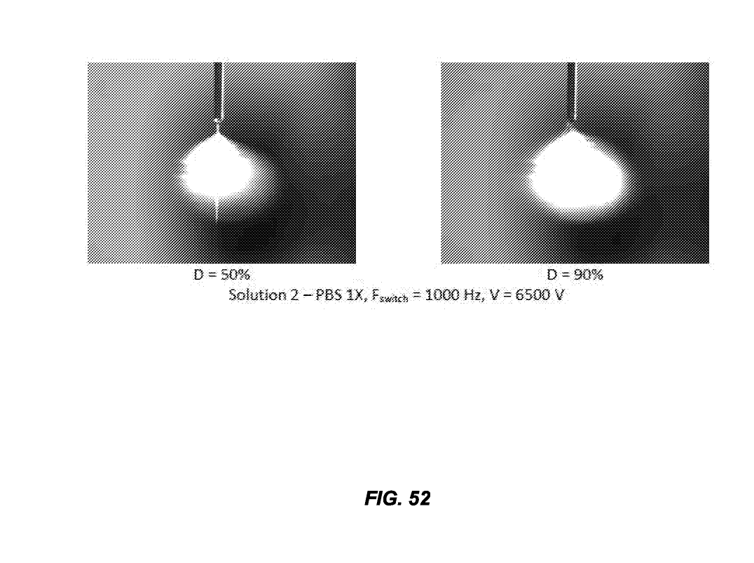

For D=50% and Fswitch=1000 Hz, the applied voltage to solution 2 was increased until a steady electrospray was induced. It was found that an applied voltage of V=6500 V was sufficient to induce a steady electrospray. The duty cycle was then increased and its effect was recorded. FIG. 52 shows a single image capture of the process at D=50% and D=90% for solution 2. As can be seen the process transitions from the cone jet mode to the multi jet mode as the duty cycle is increased from 50%-90%. At D=50%, the system shows a stable Taylor cone, at D=90%, the Taylor cone becomes chaotic and unstable. This is similar to that observed when the emitter applied voltage is increased beyond what is required to induce a stable electrospray with a constantly energized power supply.

It has been observed that a high voltage, high frequency pulsar module which can alternate a HV emitter between an energized and de-energized state can be used in induce a steady electrospray process of common solutions. The duty cycle of the HV signal can effectively control the electrospraying process at a fixed applied voltage. Above switching frequencies of 1 Hz, a duty cycle of 50% may not be sufficient to induce an electrospray of common solutions at the same applied voltage that is sufficient to induce an electrospray when supplied by an “always on” power supply source. Increasing the duty cycle of a pulsed HV signal can have the same effect on the electrospraying process of common solutions as increasing the applied voltage when supplied by a constantly energized power supply module.

Polymer and Cell Encapsulation with an Example Adaptive Electrospray

Electrospraying can include a simple, one-step technique that provides the production of polymer particles within the nano to micron size range with controlled morphology. With the use of electrospraying; cells and drug encapsulation can be achieved with no harm caused to the product of interest. The polymer coating can provide a barrier for the drug or cell against the environment, increasing shelf-life, as well as protecting the desired product from immune attack in-vivo. The polymer capsules can also be controlled to have different degrees of porosity, degradation time and morphology beneficial for cell or drug release as well as efficiency in movement in-vivo.

Alginate is used as a model polymer and electrosprayed in two different modes; plume and dripping. Both of which are useful for different types of encapsulation. An adaptive electrospray device is used to control the frequency and duty cycles of the voltage during electrospray, which is shown to control the size, morphology and electrospraying ability of the polymer solution. T-cells are then added to provide further data on the efficiency of the cell encapsulations within alginate and how the adaptive electrospray can be of use. It was seen within the results the encapsulation efficiency and size and morphology control of capsules can be altered with the adaptive electrospray

The below description provides additional data for the use of the adaptive electrospray for controlled polymer electrospraying and encapsulation of cells; characterizes the effect of frequency and duty cycles for alginate electrospraying via dripping and plume mode; and characterizes the effect of cell encapsulation within alginate electrosprayed particles, with altered frequency and duty cycles.

Sodium alginate, calcium chloride (CaCl₂), ethanol (EtOH) were all purchased from Sigma-Aldrich, Ireland. Deionised water supplied in-lab was used for solutions. T-cells were provided in-house for cell encapsulation experiments at a 10⁶ per ml pellet form.

Two solutions of alginate were prepared to investigate the effect of the adaptive electrospray on both plume and dripping mode. These two electrospray modes are used for drug and cell encapsulation and thus were both studied. For dripping mode, 2% (w/v) alginate in deionised water was left to dissolve with continuous stirring for a minimum of 12 hours. For plume mode, 2% (w/v) alginate in deionised water was also left to dissolve with continuous stirring for a minimum of 12 hours, after which 10% EtOH was added to the solution. The alginate was electrosprayed into a dish collector full of CaCL₂ used for cross-linking the alginate particles, a stock solution of 2 mM CaCl₂ in deionised water was prepared for all experiments.

The parameters for electrospraying alginate in dripping mode were; 2 cm distance, 0.3 mm tubing, 30 Gauge (G) emitter, 0.095 bar and 4 KV. The parameters for electrospraying alginate in plume mode were similar; 2 cm distance, 0.3 mm tubing, 30G emitter, 0.065 bar and 5.4 KV.

The electrospraying set up used a stainless-steel dish collector and pressure to control flow. A point-grey chameleon monochrome camera at 15 frames per second was used for electrospray observations with a laser directed at the spray for increased visualization. A light microscope was used for particle analysis with and without cell encapsulation.

For investigation into the effect of frequency and duty cycles on polymer and cell encapsulation using the adaptive electrospray; 1 Hz, 100 Hz and 1 KHz was used with duty cycles for 1 Hz set at 10%, 50% and 90 and for 100 Hz and 1 KHz duty cycles tested were 50%, 75% and 90%.

The electrospray characteristics were captured via video for roughly 4 seconds. Images described below show a snapshot for every 66 mS for an overall observation of the electrospray characteristics for 2 seconds.

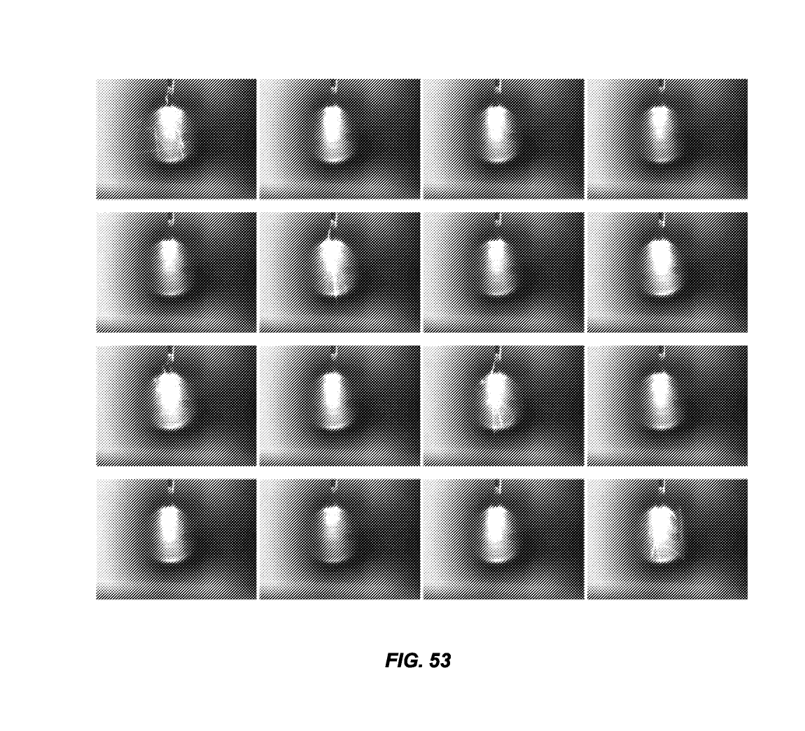

Before the frequency and duty cycles for the voltage was altered during electrospraying, a control electrospray is shown for alginate in plume mode (FIG. 53 illustrating Alginate Electrospray control at 5.4 KV, with monochrome images taken for 66 mS snapshots over 2 seconds). As can be observed the alginate particles are electrosprayed continuously within this setting.

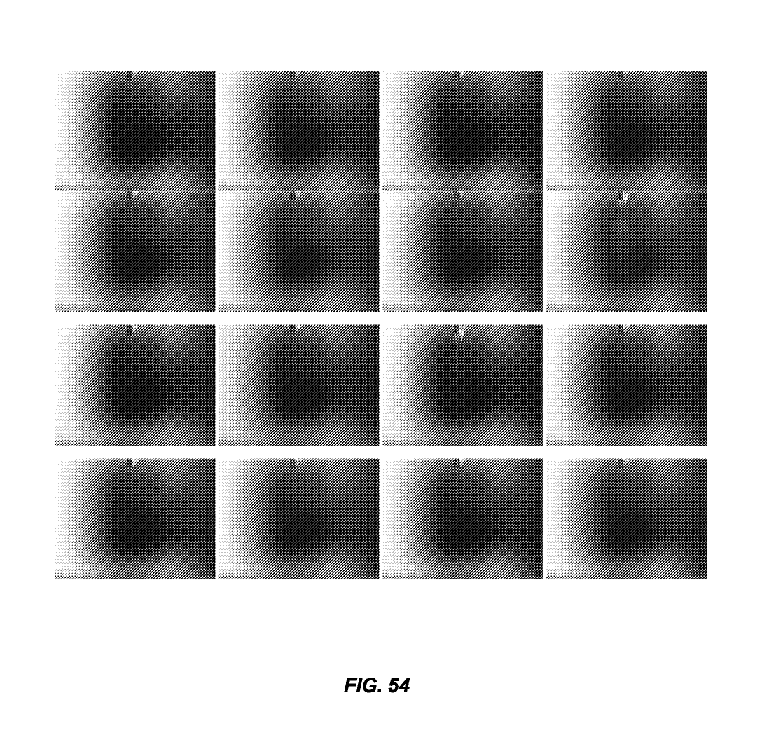

FIG. 54 illustrates alginate electrospray in plume mode using adaptive electrospray at 1 Hz 10% duty cycle. At 1 Hz, 10% duty cycle, polymer is shown to be electrosprayed once within the 2 second snapshot shown in FIG. 54. Within the 4 second video the alginate particles are electrosprayed 5 times.

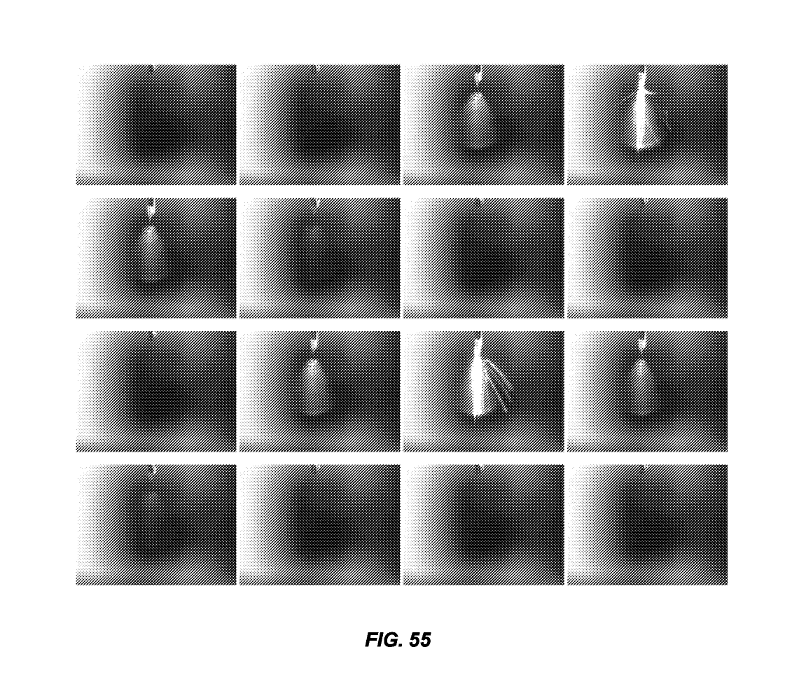

FIG. 55 illustrates alginate electrospray in plume mode using adaptive electrospray at 1 Hz, 50% duty cycle. At 50% duty cycle, the severity of the electrospray is shown to be affected by the cyclic nature of the duty cycle.

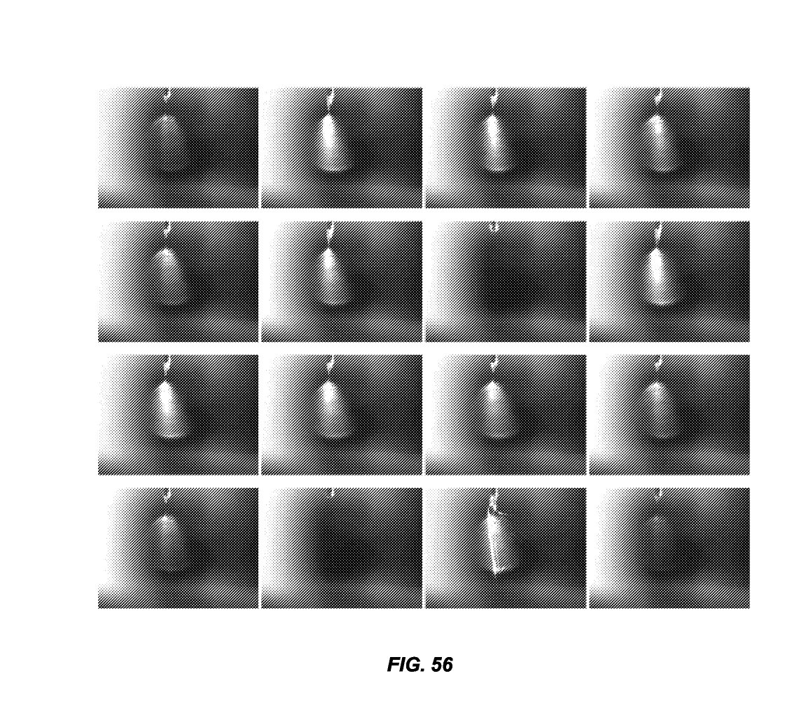

FIG. 56 illustrates 1 Hz, 90% duty cycle. At 90% duty cycle at 1 Hz, the alginate electrospray is typically on with characteristic severity of the electrospray within the 2 second snapshot shown in FIG. 56 seen to be altered through the cyclic nature.

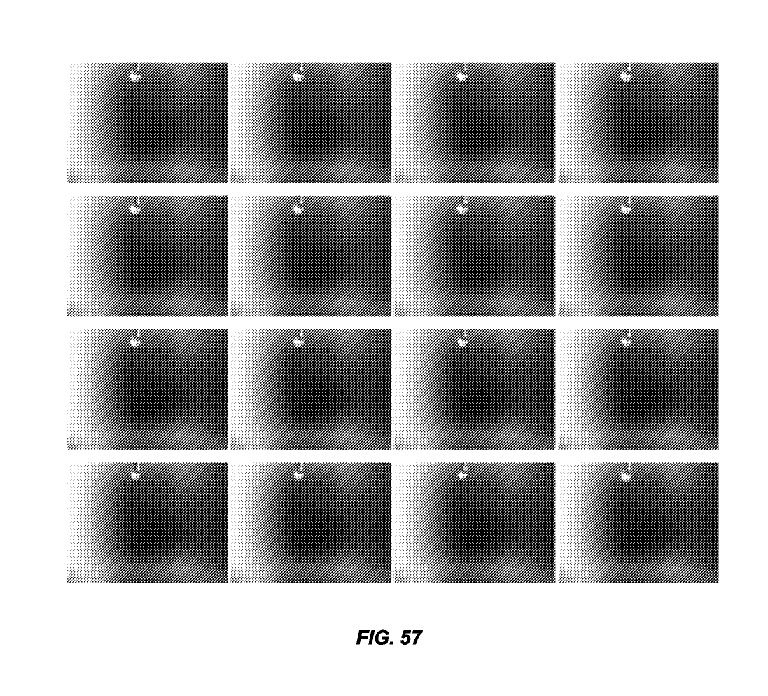

FIG. 57 illustrates 100 Hz, 50% duty cycle. At 100 Hz, 50% duty cycle the alginate electrospray was unable to be activated.

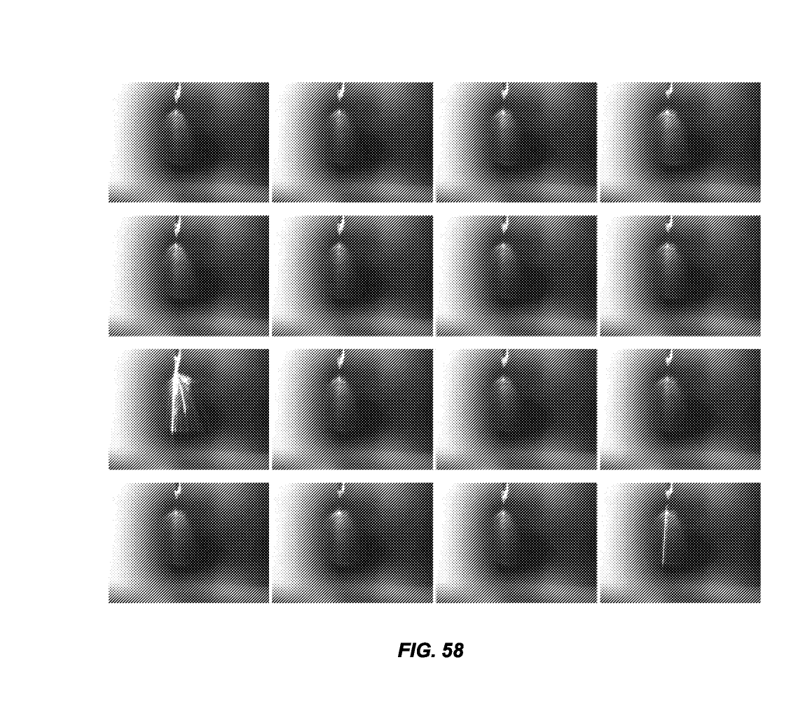

FIG. 58 illustrates 100 Hz, 75% duty cycle. With increased duty cycle at 100 Hz, the alginate could electrosprayed. Compared to 1 Hz, the alginate electrospray does not show the same severity in cyclic nature.

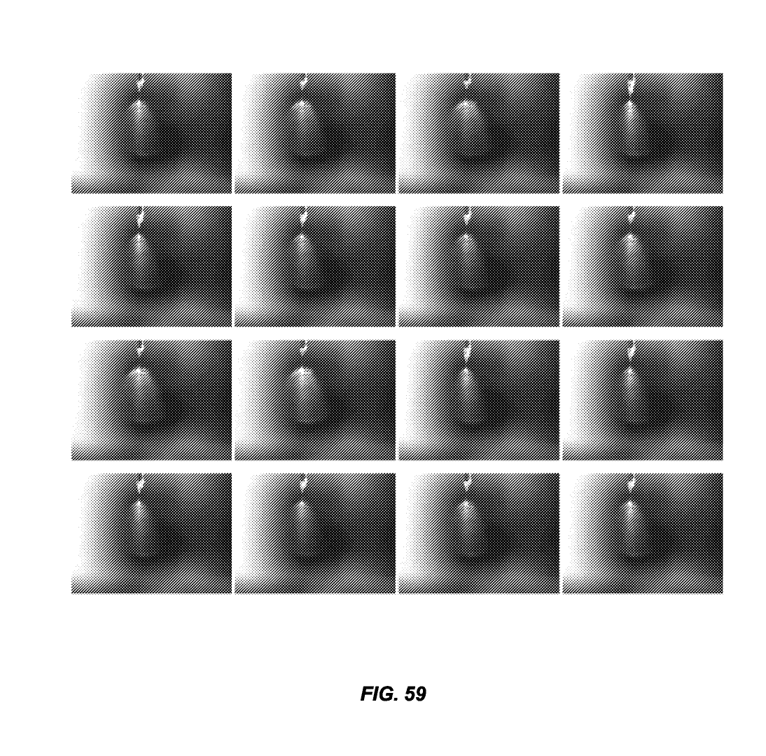

FIG. 59 illustrates 100 Hz, 90% duty cycle. At 90% the alginate electrospray is shown to be continuously activated.

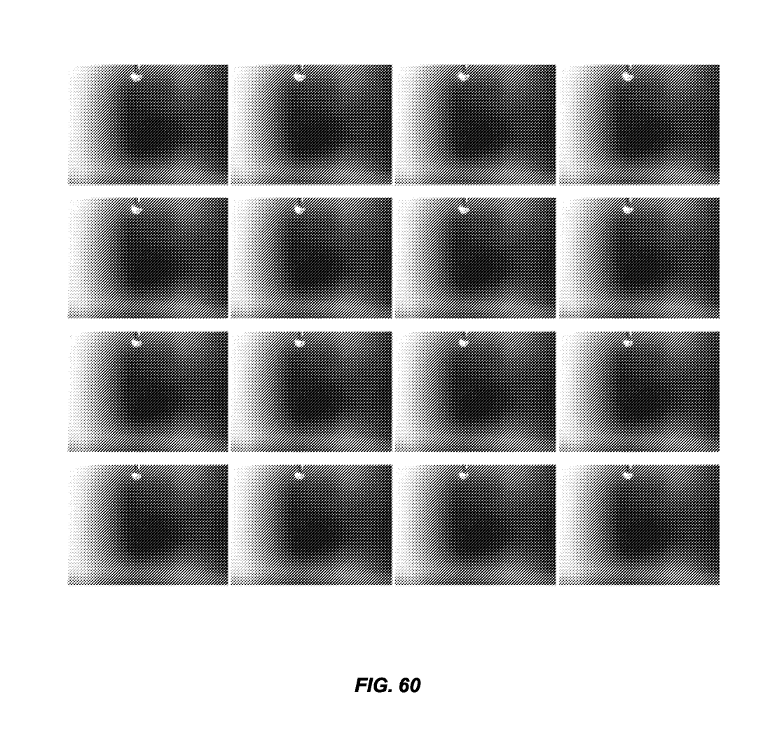

FIG. 60 illustrates 1 KHz, 50% duty cycle. As supported by 100 Hz results shown previously at the 50% duty cycle alginate is unable to electrosprayed.

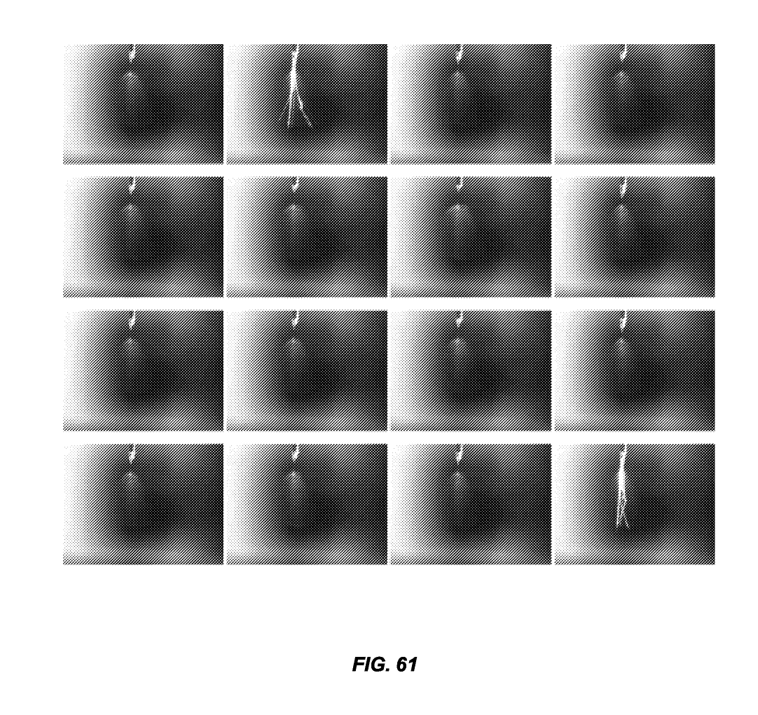

FIG. 61 illustrates 1 KHz, 75% duty cycle. At 75% duty cycle, the severity of the alginate electrospray is shown to have a cyclic nature with the electrospray continuously occurring at 1 KHz unlike at 1 Hz.

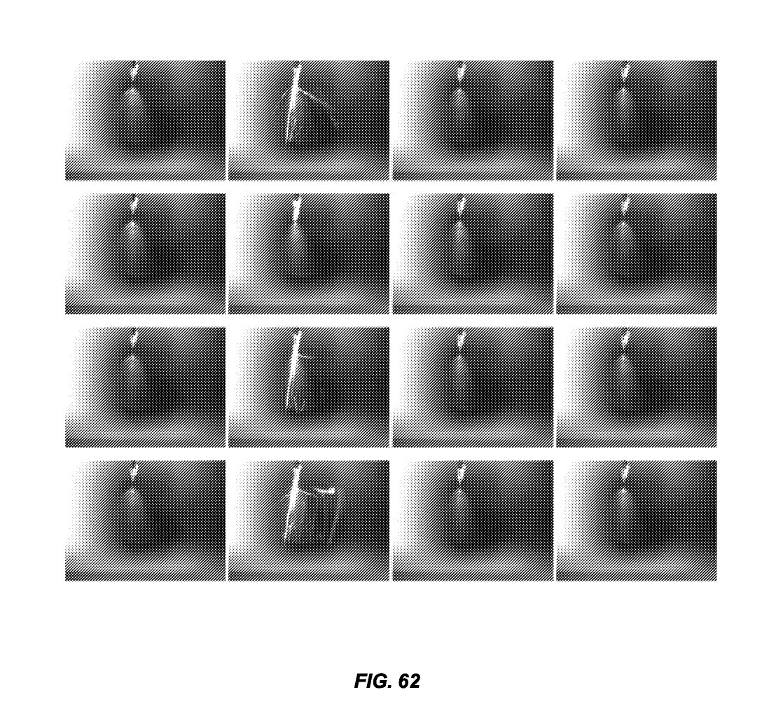

FIG. 62 illustrates 1 KHz, 90% duty cycle. At 90% duty cycle the alginate electrospray shows a stronger plume within a cyclic nature over the time.

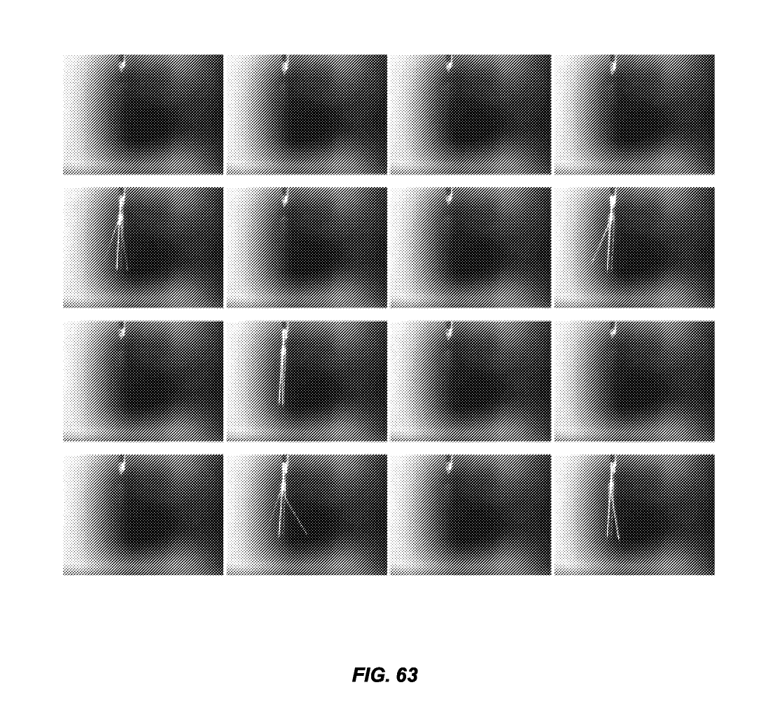

FIG. 63 illustrates Control Alginate Dripping mode. Dripping mode can be used for the encapsulation of cells (compared to plume mode electrospraying which is more typically used for drug encapsulation, although both dripping and plume mode can be used for both cell and drug encapsulation). Thus, as shown in FIG. 63, the electrospray is shown to already follow a cyclic pattern for the preparation of larger alginate particles. At 1 Hz and 10% duty cycle within the 4 second video the alginate particles are electrosprayed 5 times.

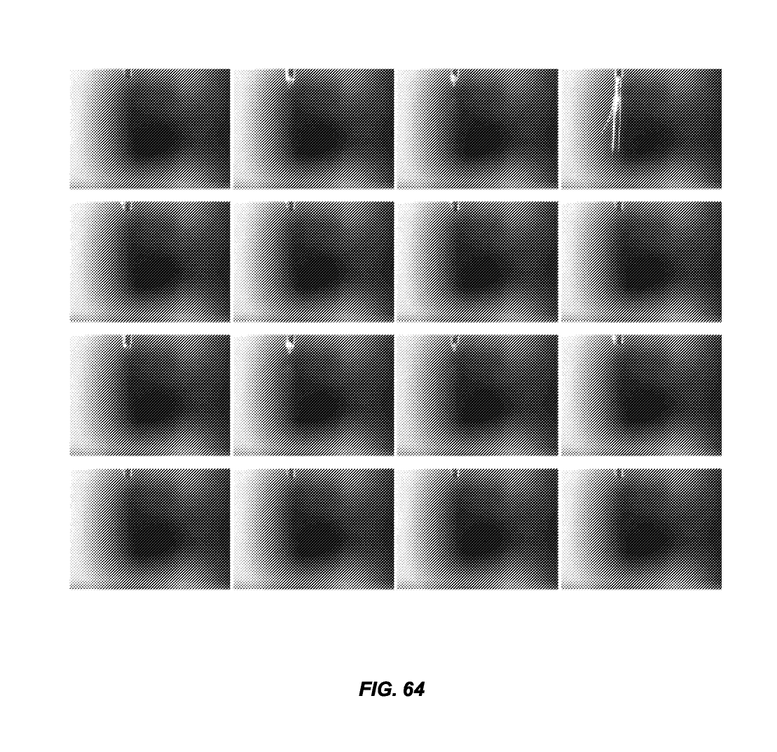

FIG. 64 illustrates 1 hZ, 50% duty cycle. At 50% duty cycle the electrospray of alginate seems to follow two severities as observed with the plume mode.

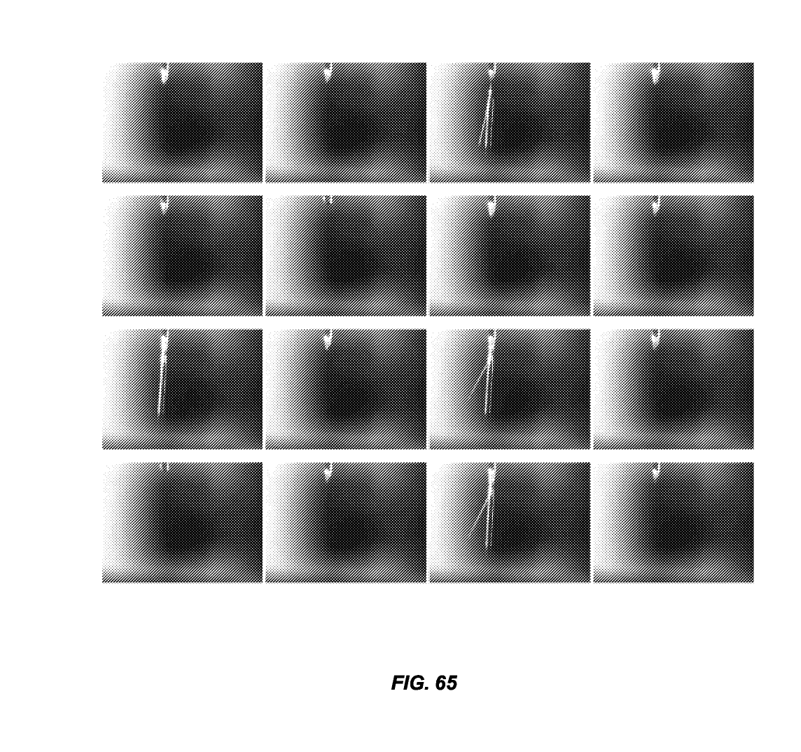

FIG. 65 illustrates 1 Hz, 90% duty cycle. At 90% duty cycle unlike with plume mode the alginate electrospray is not activated continuously.

At 100 Hz, when replaying the video the alginate drops once within the 4 seconds at 50% duty cycle. At 75% duty cycle when replaying the video, the alginate drops 0 times within the 4 seconds. At 90% duty cycle when the video was replayed showed continuous dripping.

At 1 KHz, at 50% and 70% duty cycle the alginate particles did not drop within the 4 second videos.

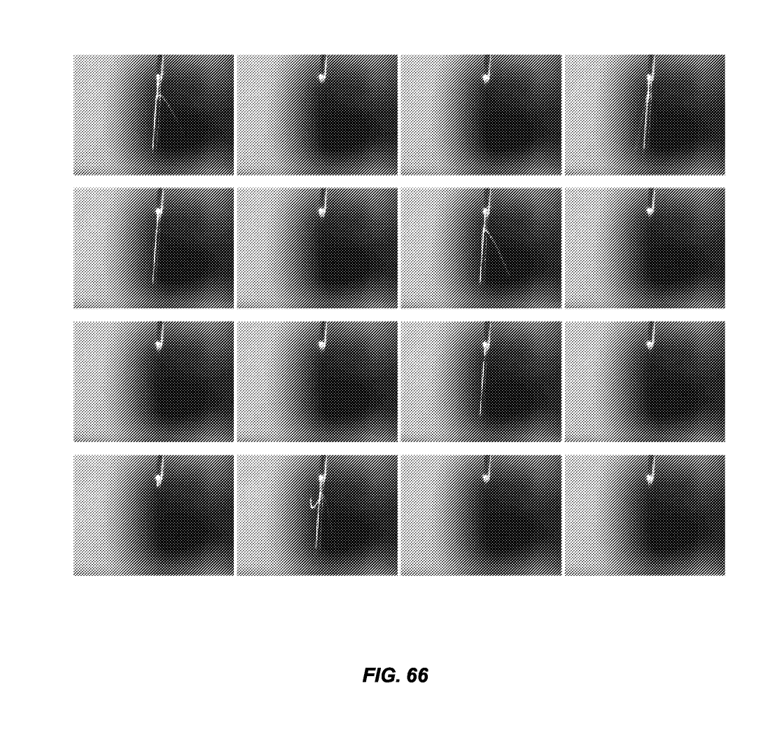

FIG. 66 illustrates 1 KHz, 90% duty cycle. At 90% duty cycle, there was a methodical electrospray of alginate within dripping mode.

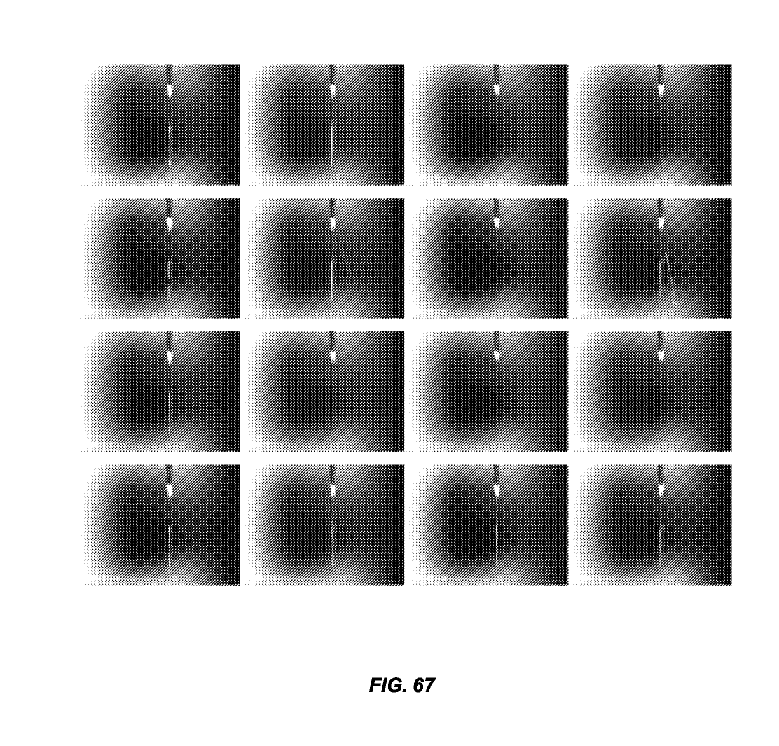

FIG. 67 illustrates T-cells in alginate solution electrospraying in dripping mode, control. The alginate particles with cells show a similar trend to the control alginate, with increased dripping over the time period.

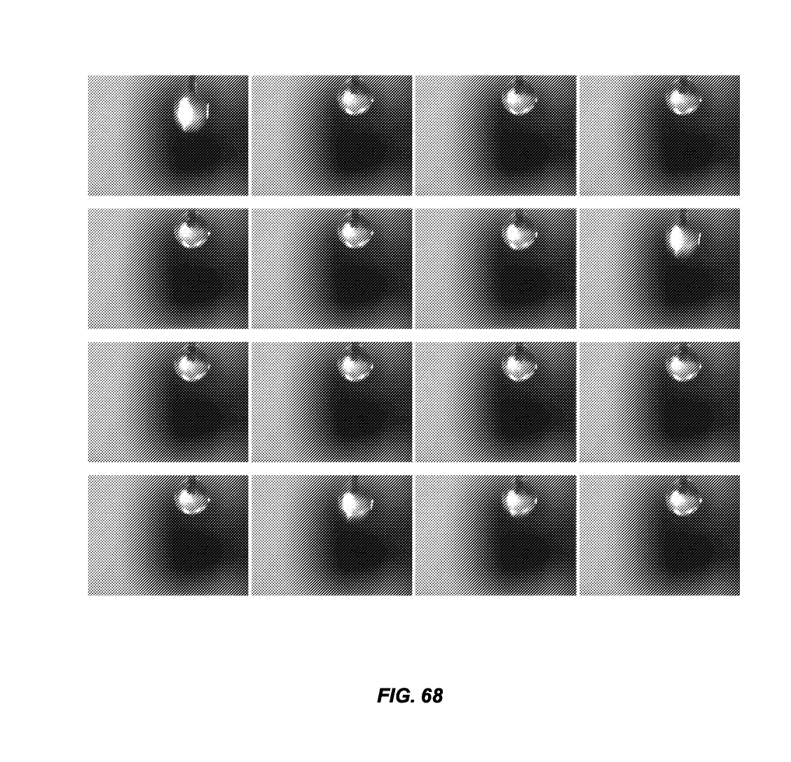

FIG. 68 illustrates T-cells in Alginate electrosprayed in dripping mode, 1 Hz, 10% duty cycle. Within the 4 second video for 10% duty cycle, the alginate particle is shown to move back and forth from the emitter without dropping.

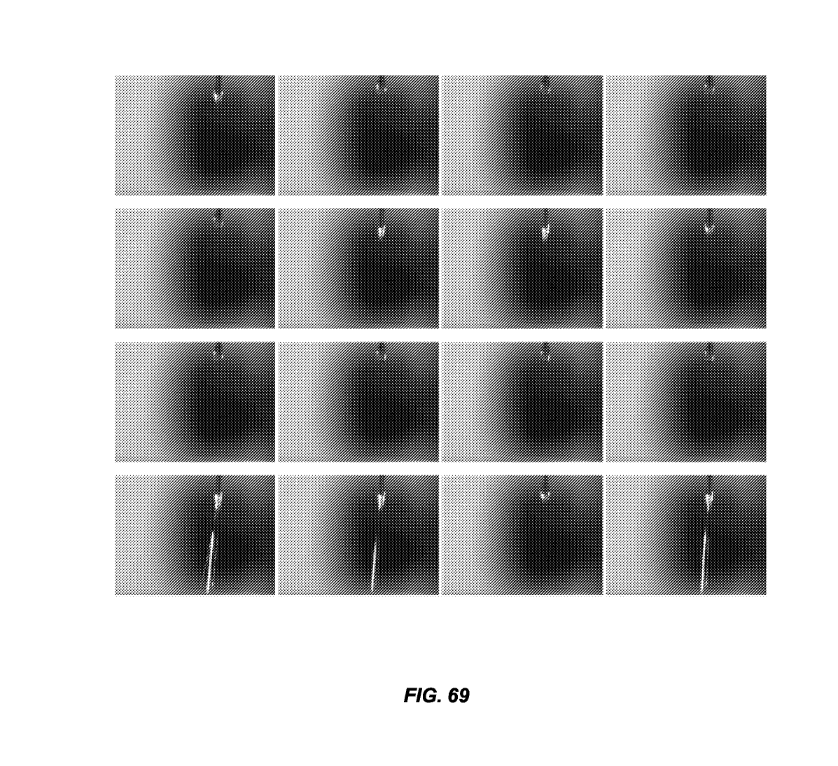

FIG. 69 illustrates T-cells in alginate electrosprayed in dripping mode, 1 Hz, 50% duty cycle. With increased duty cycle, the alginate is able to be electrosprayed with the T-cells.

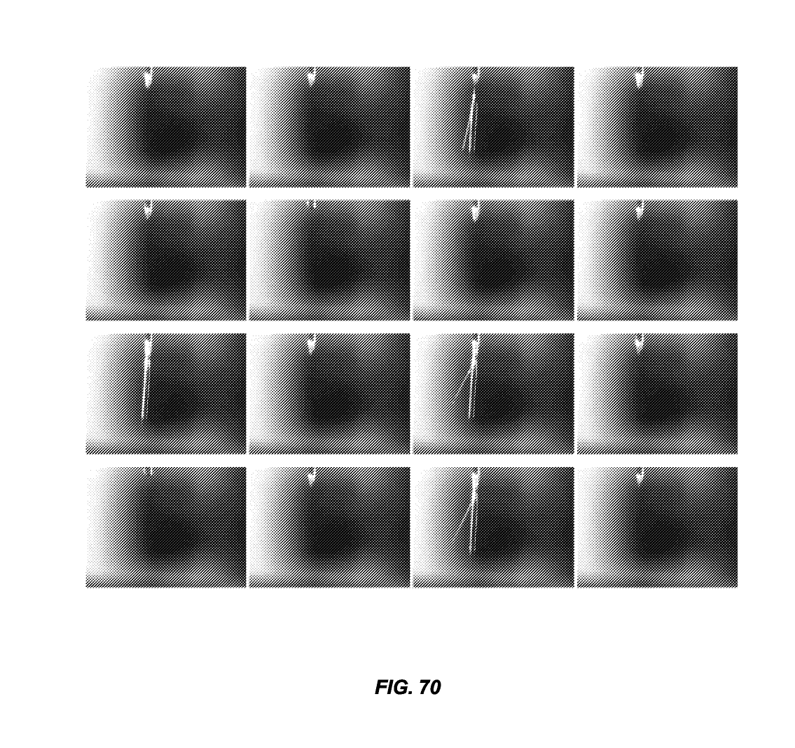

FIG. 70 illustrates T-cells in alginate electrosprayed in dripping mode, 1 Hz 90% duty cycle. Increased duty cycle, increases the amount of alginate electrosprayed within a given time.

At 100 Hz, at 50% and 70% duty cycle the alginate particles do not drop within the recorded time.

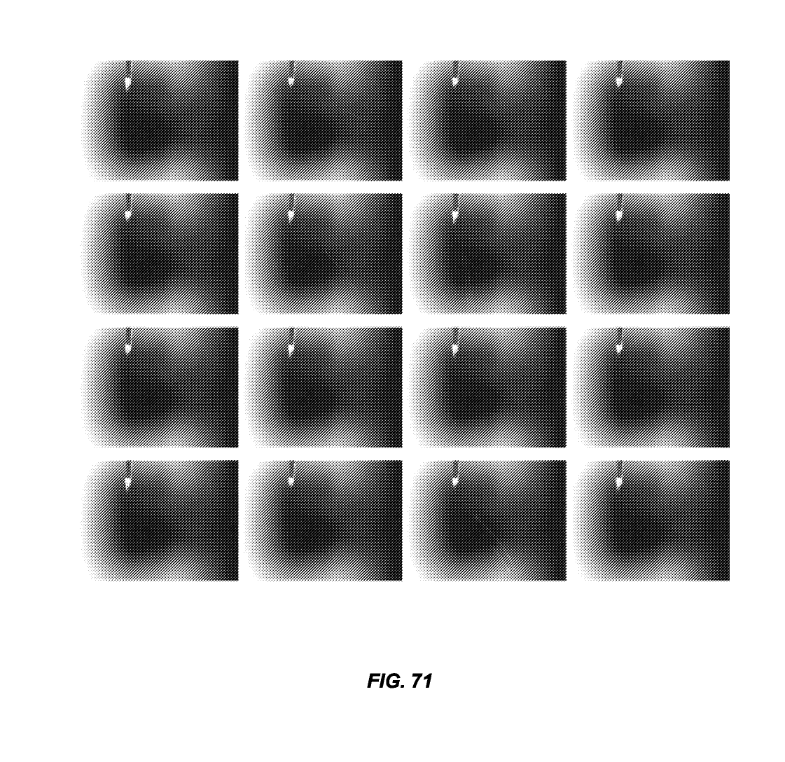

FIG. 71 illustrates T-cells in alginate electrosprayed in dripping mode at 100 Hz 90% duty cycle. At 90% duty cycle the alginate particles with T-cells are shown to be consistently being electrosprayed. At 1 KHz, at 50 and 75% duty cycle the alginate particles with T-cells did not electrospray within the 4 second videos. However, at 90% duty cycle 1 particle was electrosprayed within the 4 seconds, so the consistent electrospray at the higher duty cycles observed in the control did not occur when cells were added.

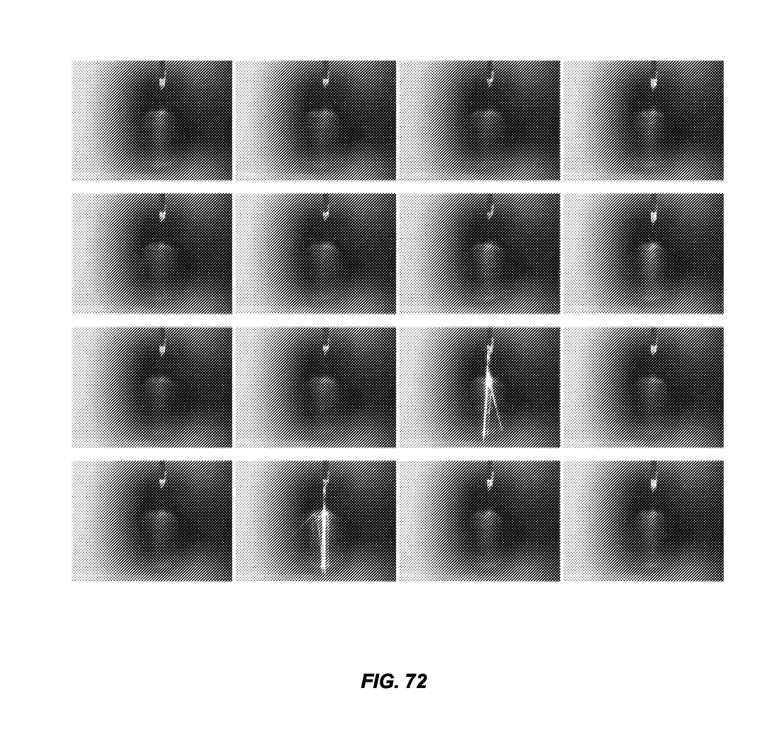

FIG. 72 illustrates T-cells in alginate electrosprayed in plume mode control. Alginate is shown to be electrospraying constantly within the control.

At 1 Hz, at 10% duty cycle the alginate particles electrosprayed 4 times within the 4 second video and showed a more dripping mode state of electrospray.

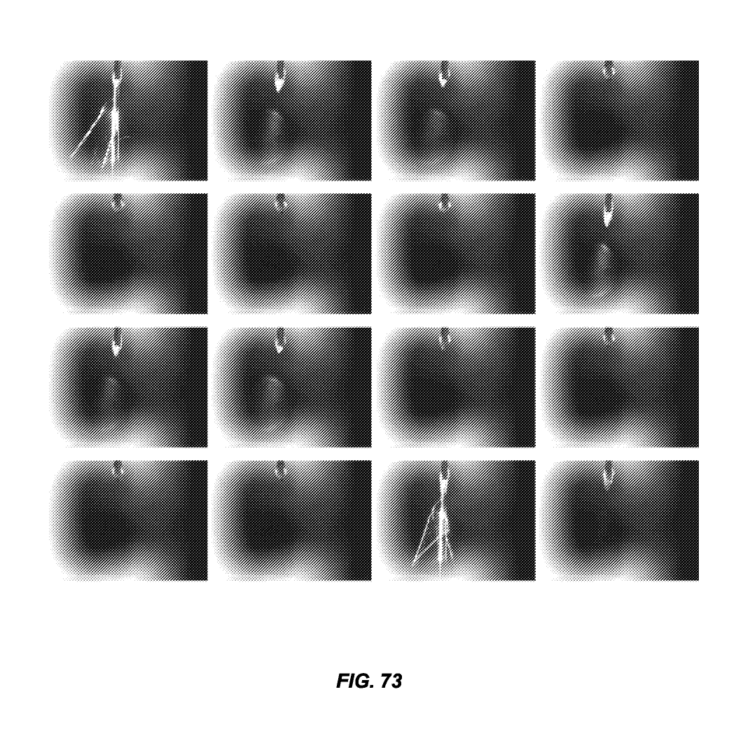

FIG. 73 illustrates T-cells in alginate electrosprayed in plume mode at 1 Hz 50% duty cycle. At 50% duty cycle, it appears to show plume and dripping mode with cells added.

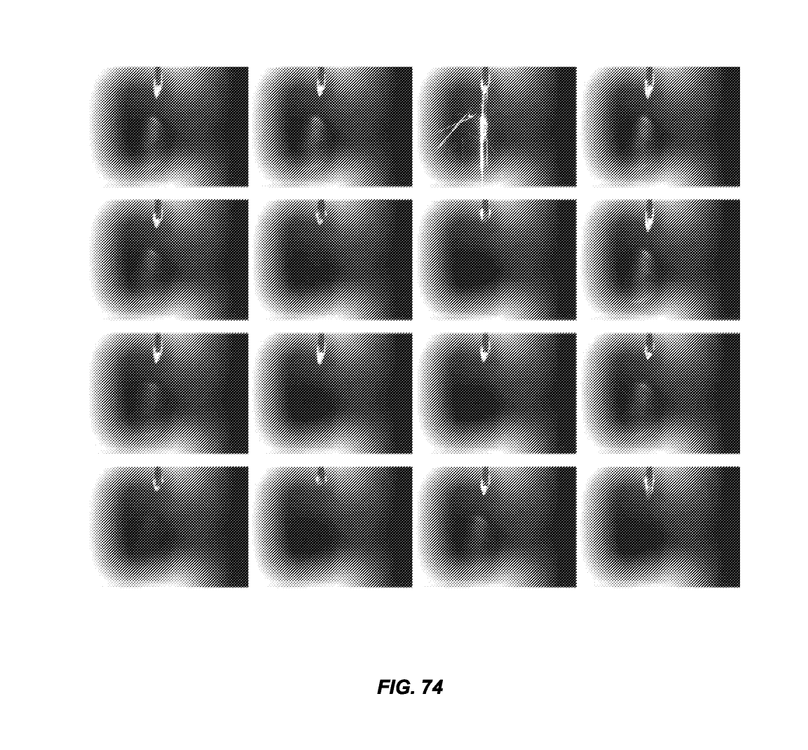

FIG. 74 illustrates T-cells in alginate electrosprayed in plume mode at 1 Hz 90% duty cycle. As supported by the 90% duty cycle which consistently was electrospraying within the video.

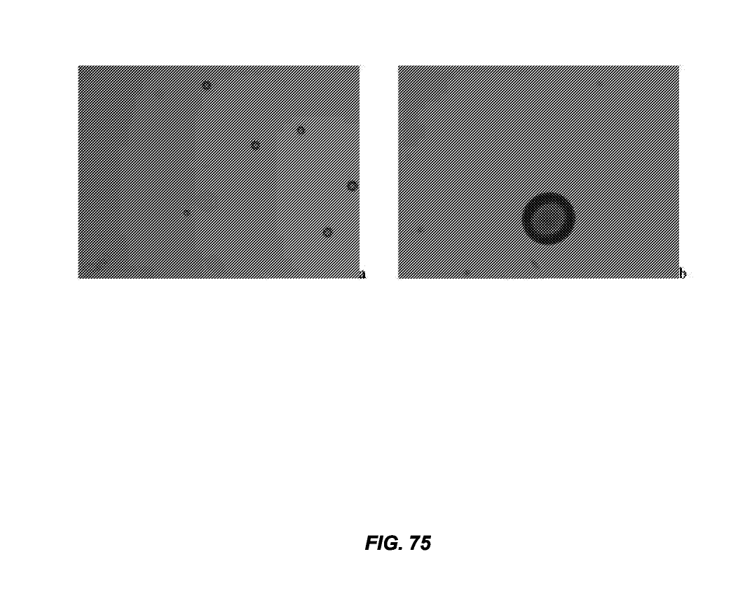

A light microscope was utilized. FIG. 75 illustrates light microscope images of alginate particles electrosprayed in dripping mode in control settings, a) 4× and b) 20× magnification. The particles when analyzed with image J are 58±9.4 μm in average size with standard deviation and spherical in morphology.

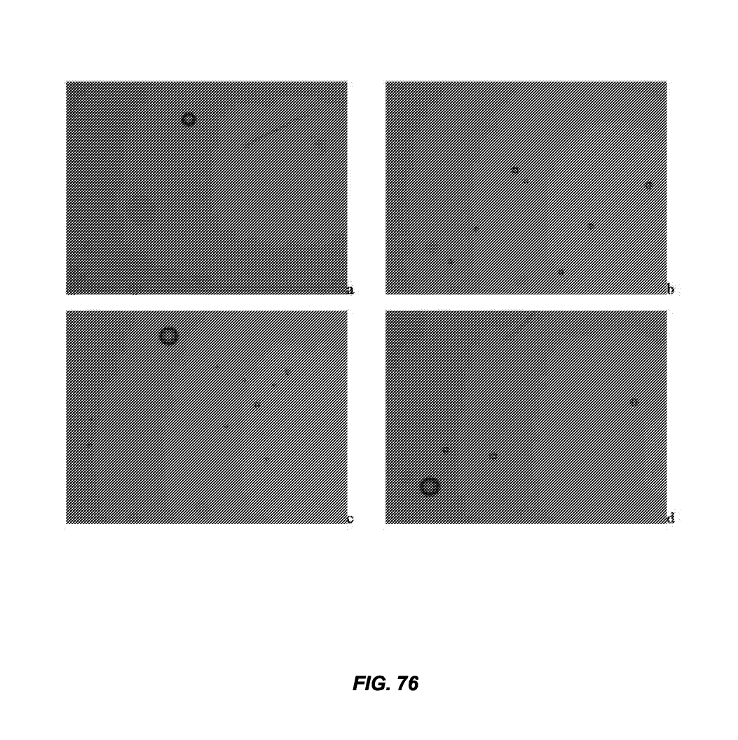

FIG. 76 illustrates light microscope images of alginate particles electrosprayed in dripping mode at 4× magnification for a) 1 Hz 10% duty cycle, b) 1 Hz 90% duty cycle, c) 1 KHz, 50% duty cycle and d) 1 KHz 90% duty cycle.

When using the example implementation of an adaptive electrospray, the average particle size with standard deviation increased to 99.5 μm for 1 Hz, 10% duty cycle and then decreased in size with increased duty cycle at 90% to 38.8±7.6 μm. When the frequency was altered to 1 KHz at 50% duty cycle the average particle size was 39.2 μm with increased duty cycle opposing the previous trend with increased average particle size observed at 72.5 μm. The particles in all conditions maintained a spherical morphology.

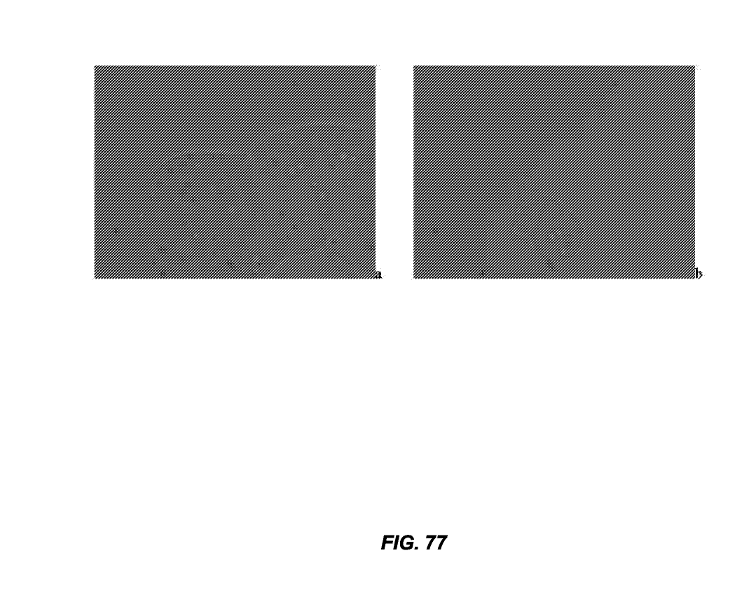

FIG. 77 illustrates light microscope images of T-cells encapsulated in alginate particles under controlled settings within dripping mode electrospray at 20× magnification.

The particles when analyzed with image J show firstly that the morphology of the alginate particles has changed from spherical to irregular shapes, observed within previous experiments and when observed with a high-speed camera it appeared to be caused by the aggregation of a smaller particle to the original primary particle. The particle size observed within these experiments was 116.4 μm.

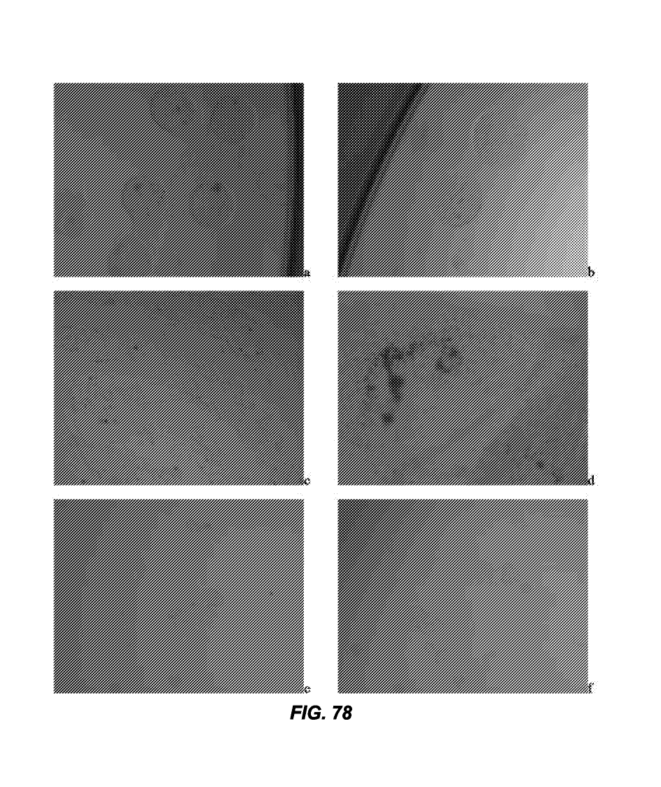

FIG. 78 illustrates light microscope images of T-cells in alginate particles at different adaptive electrospray settings in dripping mode at 4× magnification, a) 1 Hz 10% duty cycle, b) 1 Hz 50% duty cycle, c) 1 Hz 90% duty cycle, d) 1 KHz 50% duty cycle, e) 1 KHz 75% duty cycle and f) 1 KHz 90% duty cycle.

When the adaptive electrospray as used on alginate with T-cells within the solution, the electrosprayed technique showed the encapsulation of cells, with the frequency and duty cycle showing a variation in encapsulation efficiency. At 1 Hz, 10% duty cycle, the average particle size was 370±29.9 μm with cells encapsulated within spherical alginate beads; however at this duty cycle the alginate particles as shown to agglomerate to a certain degree. When the duty cycle increased to 50%, the average particle size was 472.5±24.1 μm with the particles showing no aggregation. Increasing the duty cycle further at 1 Hz, shows lack of particle encapsulation. At 1 KHz encapsulation of cells within alginate only begins to occur at 75% duty cycle the average particle size was 216.7±31 μm with entrapment in more irregular shaped particles. At 90% duty cycle, irregular particles are still observed with average particle size 225.4±54.8 μm.

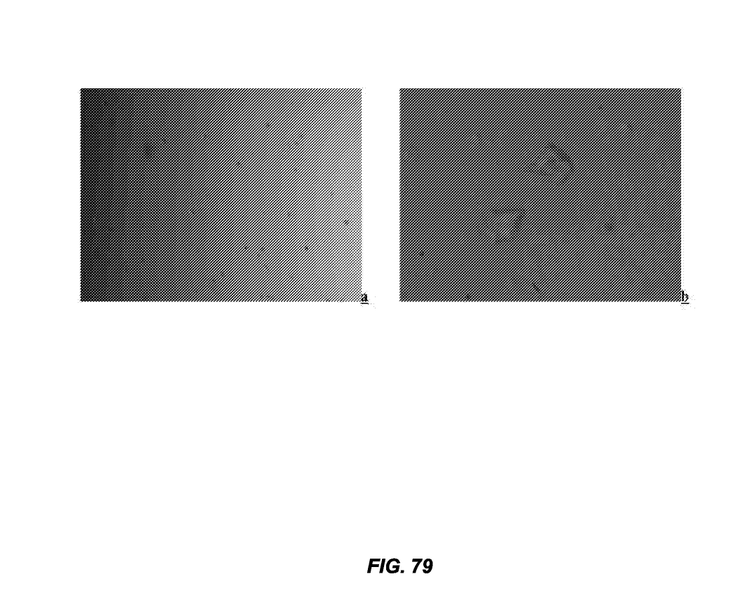

FIG. 79 illustrates light microscope images of T-cells encapsulated in alginate particles in plume mode at 1 Hz 50% duty cycle a) 4× and b) 40× magnification.

As theorized for using electrospray for cell encapsulation, plume mode provides smaller particles compared to dripping mode. It is shown that at 1 HZ, 50% duty cycle that alginate is encapsulating cells within particles in an irregular morphology.

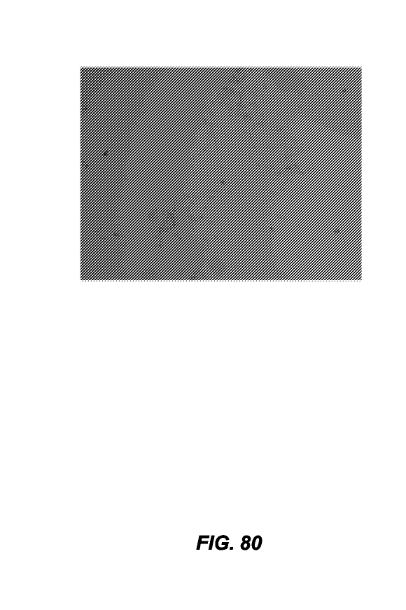

FIG. 80 illustrates light microscope image of T-cells and alginate particles in plume mode at 1 KHz 90% duty cycle 4× magnification. With increased duty cycles, it is difficult to observe the encapsulation of cells within alginate within plume mode.

The adaptive electrospray alters the electrospray characteristics of alginate within the same time periods. Higher frequencies require higher duty cycles to provide enough charge to the alginate particles to electrospray. The degree of electrospraying can be altered within the same time period with altered duty cycles. With the addition of cells, plume mode shows a mix of plume and dripping mode characteristics. The particle morphology of alginate particles with no cells is typically spherical in nature. The average particle size of the alginate particles can be altered with the adaptive electrospray. Such as at 1 Hz 10% average particle size is 99.5 μm whereas at 1 Hz 50% duty cycle average particle size is 38.8 μm.

Encapsulation efficiency can be controlled with the adaptive electrospray, shown with spherical alginate particles with cells encapsulated at 1 Hz 50% duty cycle. Whereas within the control there is lack of controlled morphology with cells encapsulated within an irregular shaped alginate. The increased frequency is shown to alter the alginate encapsulation morphology from spherical at 1 HZ to irregular at 1 KHz.

Production of Charged Spheres and/or Encapsulation of Living Cells

The instrumentation and methods described herein are useful for encapsulating, e.g., surrounding or coating, cells with a biocompatible polymer or other material by spraying cells (living eukaryotic or prokaryotic cells) and/or encapsulation material through an emitter while a charge is applied to the emitted plume of particles or droplets in a high throughput manner to generate charged particles or spheres (with or without cells inside) while eliminating run-to-run artifacts (a significant drawback of other electrospray approaches). The multi-emitter instrument generates charged spheres/particles/droplets and/or encapsulates 1×10⁶ to 10×10⁶ cells/hour or more in a consistent and reliable manner. The flow rate is in the range of 1-250 μl per minute per emitter.

In one example, cells and encapsulating material (e.g., polymer solution) are both electrosprayed through the same single emitter. In another example, the cells and encapsulating material are electrosprayed co-axially, e.g., the cells come through a first emitter and the encapsulating material comes through a second co-axial emitter and the Taylor cones coincide, and a plume develops thereby producing a cell or plurality of cells encapsulated or surrounded by the encapsulating material. In yet another example, the instrument is used to generate charged particles or spheres of the encapsulating material, e.g., the encapsulating material is electrosprayed through an emitter to generate charged particles, e.g., alginate beads. Following that process, the alginate particles are permeabilized and contacted with cells such that the cells gain entry into the alginate beads, resulting in cell encapsulation. In the latter example, large quantities of consistently-produced alginate spheres with relatively uniform size within 20% are formed using the instrumentation are collected and subsequently loaded with cells.

Encapsulation materials include compounds and compositions that are biocompatible, e.g., cytocompatible, such as those that are soluble and/or miscible in pharmaceutically-compatible excipients or solutions such as water or buffers such as phosphate buffered saline. Exemplary polymers for living cell encapsulation or non-cell containing sphere formation include alginates (modified or unmodified), Polycaprolactone (PCL), Polycaprolactone (PLC), Poly(DL-lactide-co-glycolide (PDLG), Thermoplastic Polyurethane (TPU) (selectophore), Thermoplastic Polyurethane (TPU) (Elast-Eon), Gelatin, Polyvinylpyrrolidine (PVP), Polyvinyl acetate (PVA), and/or Polyethylene glycol (PEG), including co-polymers thereof. Solution electrosprayed polymers include BSA (bovine serum albumin), Riboflavin, Mannitol, Chitosan, Poly(lactic-co-glycolic acid) (PLGA), Polyacrylic acid, Poly(glycerol sebacate) (PGS), and/or Alginate, including co-polymers thereof. For example, bioelectrospray of A549 cells encapsulated in alginate beads was accomplished using the instrument and methods described herein. Other useful polymers include Polycaprolactone (PCL) (of multiple molecular weights), Modified Polycaprolactone (PCL), and/or Polydioxonone (PDO). Encapsulating materials may also include hydrogels, ceramics, metals and other plastics.

Triazole containing analogues of alginate spheres/particles has been generated using an electrostatic droplet generator such as Spraybase. The instruments and methods described are useful using the same and similar polymers with a significant improvement in the biofabrication, e.g., increased scale, improved consistency of particle size, and improved consistency of particle charge. For example, Triazole-thiomorpholine dioxide (TMTD) alginate is used to encapsulate living cells for implantation into the body. This polymer resists implant fibrosis in both rodents and nonhuman primates, e.g., TMTD alginate-encapsulated, e.g., stem cell-derived beta cells (SC-β cells) provided long-term glycemic correction and glucose responsiveness without immunosuppressive therapy in immune-competent diabetic C57BL/6J mice.

TMTD alginate synthesis has been described and can be carried out as follows. Briefly, 3.5 g of 4-propagylthiomorpholine 1,1-dioxide (1 equiv., 20 mmol) is added to a solution of 2.5 g Tris[(1-benzyl-1H-1,2,3-triazol-4-yl)methyl]amine (TBTA) (0.2 equiv., 4 mmol), 750 μl triethylamine (0.5 equiv., 10 mmol), 250 mg Copper(I) iodide (0.06 equiv., 1.3 mmol) in 50 ml methanol. The mixture is cooled to 0° C., and 5.25 ml of 11-azido-3,6,9-trioxaundecan-1-amine (1 equiv., 20 mmol) was added. The reaction is agitated overnight at 55° C., and the solvent was removed under reduced pressure. The crude reaction is purified by reverse-phase (water/acetonitrile) flash chromatography on a C18 column, yielding purified TMTD amine. This product is then reacted with ultrapure alginate as follows: 1.5 g of UP-VLVG (1 equiv., >60% G, ˜25 kDa MW, NovaMatrix cat. #4200506) was dissolved in 45 ml of water and 675 mg of 2-chloro-4,6-dimethoxy-1,3,5-triazine (CDMT, 0.5 equiv.), and 840 μl of N-methylmorpholine (NMM, 1 equiv.) was added. Then 7.65 mmol of the TMTD amine is dissolved in 22.5 ml acetonitrile and added to the mixture. The reaction is stirred overnight at 55° C. The solvent is removed under reduced pressure and the solid material is dissolved in water. The solution is filtered through a pad of cyano-functionalized silica (Silicycle), and the water was removed under reduced pressure to concentrate the solution. It is then dialyzed against a 10,000-Da molecular weight cutoff (MWCO) membrane in deionized water overnight. The water is removed under reduced pressure to give the functionalized alginate.

Fabrication of alginate hydrogel spheres and cell encapsulation using the described instrument and method yields superior spheres/particles compared to earlier devices and methods. The pre-spray preparation of reagents need not be substantially modified prior to biofabrication using the emitter array device described herein. For example, the following reagent preparations can be employed. Prior to sphere fabrication, buffers were sterilized by autoclaving, and alginate solutions were sterilized by filtration through a 0.2-μm filter. Aseptic processing was implemented for fabrication by performing capsule formation in a type II class A2 biosafety cabinet to maintain sterility of manufactured microcapsules/spheres for subsequent implantation. Instead of an electrostatic droplet generator connected to a luer lock syringe and needle, e.g., 18 or 25 gauge needle assembly, the mixture, e.g., alginate mixture is processed using a multi-emitter, current-regulated electrospray instrument as described above. The process consistently yields particles (cells encapsulated in polymer of choice) in the size ranges of 5 μm-3 mm, e.g., 5-500 μm, 5-10 μm, 10-100 μm, 100-1,000 μm, and/or 500 μm-3 mm in diameter.

The methods are suitable for encapsulating a wide variety of cells including mesenchymal stem cells, immune cells such as T cells (including CAR T cells) and B cells, as well as cells used for tissue repair or regeneration such as pancreatic β cells. Clinical applications include cell therapy, controlled drug release, tissue regeneration and immune-isolation of therapeutic cells.

For example, earlier methods were used to process pancreatic β cells. After the capsules are formed, they are then collected and then washed with HEPES buffer (NaCl 15.428 g, KCl 0.70 g, MgCl₂.6H₂O 0.488 g, 50 ml of HEPES (1 M) buffer solution (Gibco, Life Technologies, California, USA) in 2 liters of deionized water) four times. The alginate capsules are left overnight at 4° C. The capsules are then washed two times in 0.8% saline and kept at 4° C. until use. To solubilize alginates, SLG20 (NovaMatrix, Sandvika, Norway, cat. #4202006) was dissolved at 1.4% weight to volume in 0.8% saline. TMTD alginate was initially dissolved at 5% weight to volume in 0.8% saline and then blended with 3% weight to volume SLG100 (also dissolved in 0.8% saline) at a volume ratio of 80% TMTD alginate to 20% SLG100. 0.5-mm spheres were generated with a 25G blunt needle, a voltage of 5 kV and a 200 μl/min flow rate. For formation of 1.5-mm spheres, an 18-gauge blunt-tipped needle was used with a voltage of 5-7 kV. Immediately before encapsulation, the cultured SC-β clusters were centrifuged at 1,400 r.p.m. for 1 min and washed with calcium-free Krebs-Henseleit (KH) Buffer (4.7 mM KCl, 25 mM HEPES, 1.2 mM KH₂PO₄, 1.2 mM MgSO₄×7H₂O, 135 mM NaCl, pH=7.4, =290 mOsm). After washing, SC-β cells were centrifuged again and all of the supernatant was aspirated. The SC-β pellet was then resuspended in the SLG20 or TMTD alginate solutions at cluster densities of 1,000, 250, and 100 clusters per 0.5 ml alginate solution. Spheres were crosslinked using a BaCl₂ gelling solution, and their sizes were controlled as described above. Immediately after cross-linking, the encapsulated SC-β clusters were washed four times with 50 ml of CMRLM medium and cultured overnight in a spinner flask at 37° C. before transplantation. This method was characterized by an inevitable loss of SC-β clusters during the encapsulation process. The methods described herein effectively and consistently yield encapsulated cells at high throughput/scale in a reliable one step electrospray emission process.

Characterizing the Effect of Ambient Conditions for Electrospraying

Electrospraying is a highly advantageous technique for the versatile preparation of nano- to micron-sized particles. One area of growing interest is within drug delivery and the preparation of particles within an industrial scale setting. For this application, the repeatability of particle production; with regards to particle size and morphology, can be finely controlled. To electrospray (ES) particles, three parameters can be optimized: solution, process and ambient. There is little investigation into the use of ambient parameters compared to the other two categories, typically due to the additional instrumentation required. Using a commercial climatic control chamber, temperature and humidity within the typical ambient ranges were systematically investigated. The effect on particle size, shape and surface morphology were analyzed for water-soluble, organic and inorganic solvent systems with FDA approved polymers; Polyvinyl pyrrolidone (PVP), Polycaprolactone (PCL) and Poly(D,L-lactide-co-glycolide) (PLGA). The results highlighted the significance of ambient conditions on all three solvent systems. Each polymer-solvent interaction was shown to follow different ES trends under ambient conditions. In inorganic solvent systems, with increased temperature and humidity, the size and shape of the particles were significantly affected, with increased porosity in PCL particles and shape control in PLGA observable. Within PVP water soluble and organic based systems, there is a significant shift from ES to electrospinning with increased temperature and humidity. The use of PVP within the different systems also highlights the importance in the interaction between the polymer itself with the solvent. The methodical testing of each polymer system in different ambient conditions provided support for the necessity in controlling ambient conditions for ES to maintain repeatability of the technique.

Electrospraying (ES) is a branch of the electrohydrodynamic process which atomises a solvated polymer using electrical forces. The technique provides control over the size and shape of fabricated nano- to microparticles. Due to this size range, there is a growing interest and application within healthcare; particularly in nanotechnology based pharmaceuticals. The coating of drugs and proteins of interest with a polymer layer has been shown to provide an additional protection within the body for controlled drug release and efficacy.

As ES provides versatility in the polymer type that can be used with control over size and shape in a one-step process; improved drug delivery systems can be prepared, which would surpass the capability of current techniques such as spray drying, emulsification or polymerization. To establish the technique within an industrial setting; scalability and reproducibility can be optimized. Here, reproducibility of ES particles are investigated, with a specific focus on controlled size and morphology of particles. Researchers typically focus on 2 of the 3 main parameters; solution and processing, with sparse research into the 3^{rd} parameter; ambient conditions. Researchers still note the need for a systematic study of ambient conditions on a wider range of electrosprayed polymer particles.

Within the electrospraying process, control over the size of the particles is typically affiliated with an interplay between the emitter size, flow rate, solvent type with regards to volatility and conductivity and the polymer concentration and molecular.

The primary particle is the main particle desired for the sample deposition. Depending on the concentration of the polymer, the degree of entanglement caused by solubility within the solution and volatility of the solvent; the viscosity and concentration is altered at the tip and during the ES process. If there is a higher amount of chain entanglement coupled with a low rate of solvent evaporation, particles are more likely to form more dense, uniform particles. Whereas if the chain entanglement within the polymer solution is more sporadic with a high rate of solvent evaporation the particles would show a larger polydispersity. Within the latter case, there is a higher likelihood of forming satellite particles, which are the breakdown of the primary droplets by a factor of 0.2 to 0.5, as well as offspring droplets which are typically classified as debris polymer particles caused by the fission of the primary droplet. Although the effect of temperature and humidity is not detailed within a systematic way compared to the other factors for particle size control. The use of higher temperatures and lower humidity's would show a knock-on effect on these process and polymer parameters.

For electrosprayed morphology control; relative humidity and temperature is more commonly taken into account within research, along with the previous polymer and processing parameters highlighted for their effect on particle morphology. Polymer morphology can be divided into two categories; primary and secondary. In the primary morphology, the overall shape of the particles, such as; spherical, doughnut, shells, lanterns, and cylindrical is considered. As well as the transition between the different states of the electrohydrodynamic process; from electrospraying to electrospinning. Whereas the secondary morphology concentrates on the external surface morphology of the electrosprayed particles with regards to porosity and roughness, as well as additional details coming forth on the internal porosity caused through the technique, namely due to solvent type used and polymer-solvent interaction.

Effect of temperature and humidity on ES particles. Temperature and humidity μlay a significant role on the size and morphology of the particles produced. As electrospinning is a more developed electrohydrodynamic process, additional supporting material on the implications of ambient conditions on fiber size and morphology were reviewed. As with electrospraying, the systematic effect of ambient conditions on electrospun fibers is also quite sparse. However, as noted with electrospraying, the parameters that significantly affect the electrospun fiber size and shape are also; polymer molecular weight and concentration and solvent type and volatility. With the influence on primary and secondary surface morphology affecting each polymer system differently.

The significance of the particular polymer and solvent when electrosprayed or electrospun at a range of humidity's and temperatures, highlights the need for a more detailed understanding of the polymer-solvent interaction, to predict and optimize the ambient conditions appropriately for finer control on the electrohydrodynamic process. Vrieze et al, noted that fibers electrospun from cellulose acetate (CA) in acetone and N,N-dimethylacetamide (DMAc) at increased humidity were larger in size; whereas fibers electrospun from PVP in ethanol trended towards a smaller size with increased humidity. They noted that there was an intricate relationship between the polymer and its impact on the solvent evaporation rate, through chemical and molecular interaction between the two. There is also a significant interplay between the three parameters; process, polymer and ambient for tight control of morphology. This has been shown, whereby temperature was maintained at 20° C., solvent type, TFE and presence of polymer, PLGA, through the use of flow rate, showed the different effects on the evaporation rate of TFE during the process. Causing the presence of different primary morphologies from beaded fibers, elongated particles and spherical particles. The ability to control primary and secondary morphology is an additional parameter that can be controlled, especially with regards to drug delivery, as variation in porosity influences the pharmacokinetics of the drug released, as well as the integrity of the ES particles and degradation rates.

The relationship between temperature and humidity is therefore of importance to optimize within the ES polymer system. At present the exact mechanism underlying the alteration in morphology and production of controlled porosity is not fully understood. However, two theories are commonly proposed within the literature with regards to temperature and humidity for their effect on the morphology of the resultant polymer particles or fibers. These are phase separation and breathe figure theory, and typically consider the interaction between the solvent type and volatility, polymer, and temperature account for the alteration in morphology.

In phase separation, there are 4 main mechanisms that are proposed; ‘thermally induced phase separation, immersion precipitation, air casting of the polymer solution and precipitation from the vapor phase. As thermodynamic instability is the driving force of phase separation; reduction in temperature, loss of solvent and increase in non-solvent (water) during electrospraying affect the degree of instability. With higher temperature, solvent within the polymer solution evaporates at a faster rate. This results in a temperature decrease of the polymer solution and causes non-solvent to diffuse into the particle spray. Depending on the polymer miscibility with water vapor in the atmosphere, this can cause the polymer to precipitate at a faster rate, resulting in larger structures or if the polymer is miscible with water. It also causes increased conductivity of the polymer and reduced surface tension, resulting in further stretching or increased atomisation and thus smaller particles or fibers.

In breath figure theory, at a threshold atmospheric humidity, water condenses from the gas phase onto the surface of the atomised particles or fibers within the jet stage of the technique. Depending on the solvent volatility, pores can then be produced through the evaporation rate of the solvent from the polymer particle in relation to the water evaporation rate from the polymer particle or fiber. This results in a patterned porous surface.

The lack of research within this area could be due to the additional instrumentation required to provide a closed chamber with controlled temperature and humidity. Temperature and humidity therefore has not been extensively evaluated, however what has been highlighted within the literature is the intricate complexity and control required even within the ambient temperature and humidity ranges that are present in laboratories as well as when upscaling for industrial use.

Described below is an investigation into the effect of temperature and humidity at controlled points within the ambient ranges with the use of a commercial closed temperature and humidity chamber. To evaluate the relationship between the polymer and solvent type and volatility, three different FDA approved polymers; polyvinyl pyrrolidone (PVP), Polycaprolactone (PCL) and Poly(D,L-lactide-co-glycolide) (PLGA), all of which would be beneficial for different drug delivery systems, were tested.

The polymers used were Poly vinylpyrrolidone (PVP) mw 55,000, Polycaprolactone (PCL) mw 70-90,000, both purchased from Sigma-Aldrich, Ireland and Poly (D,L-lactide-co-glycolide) (PDLG 7520A) purchased from Purasorb, UK. Solvents used to prepare polymer solutions for testing were ethanol (EtOH), 2,2,2-trifluoroethanol (TFE), chloroform, chlorobenzene, purchased from Sigma-Aldrich, Ireland and deionised water, filtered in-lab.

PVP solutions were prepared at room temperature by dissolving PVP pellets at a 50%, 25% and 12.5% (w/v) concentration in either DW, EtOH or a 1:1 ratio (v/v) of DW:EtOH respectively. The solutions required a minimum of 12 hours to dissolve into solution. For PCL solutions, 5% (w/v) concentration of polymer in chloroform:chlorobenzene at a 80:20 (v/v) ratio was prepared at room temperature and left to stir on a magnetic plate for a minimum of 3 hours. For PLGA solutions, 7% (w/v) concentration polymer was prepared in TFE and left to stir at room temperature for a minimum of 3 hours before testing.

For the methodical testing of temperature and humidity within ambient ranges typically present, a commercial climatic control chamber, designed by Spraybase (Avectas, Dublin Ireland) was used. Temperatures tested for each electrosprayed polymer were at 20, 30 and 40° C.; with humidity altered at each temperature time point at 30, 40 and 50%.

The temperature and humidity chamber used a pressure driven system for controlling flow rate and a monochrome camera was present within the set-up to monitor the polymer electrospraying characteristics continuously throughout the experiment for improved observations.

All polymer solutions used 0.3 mm tubing and a 22G emitter. The distance, flow rate and voltage however were altered for the solutions depending on optimized parameters. For PVP solutions a 10 cm distance, 0.1-0.15 bar pressure and 10-11 KV was used. For PCL and PLGA solutions a 15 cm distance was used, with PLGA solutions requiring a 0.06 bar pressure and 12 KV; whereas PCL used a 0.376 bar pressure and 6-7 KV.

The electrosprayed particles were deposited onto aluminum foil on a static collector within the chamber. The foil was then removed and 2 cm×2 cm samples were cut from the electrosprayed foil to be characterized using a Jeol SS-5500 benchtop scanning electron microscope (SEM). A minimum of 6 spots were scanned for each sample. The particle size was measured using Image-J (NIH software) to calculate the particle diameter of the SEM images and a minimum of 50 particles were measured for each parameter.

The intricate relationship between temperature and humidity with the polymer and solvent system has been shown to affect the resulting size and morphology of ES particles to different degrees dependent on the volatility of the solvent and the interaction with the polymer.

PVP was solubilized in three different solvent systems; deionised water (DW), ethanol (EtOH) and a 1:1 ratio of DW and EtOH. Preliminary experiments were then conducted (Appendix. 1) to obtain threshold concentrations that produced electrosprayed particles under the same ambient conditions; 20° C., 30% relative humidity (RH). These concentrations were then used for the tests outlined below. The use of three different solvent systems with the same polymer was to evaluate the significance of the interaction between the polymer with the solvent. After which, further investigation of more volatile solvents with different polymer systems, still of relevance to drug delivery, were evaluated, to confer any identifiable trends of particle morphology with temperature and humidity.

As ES particle size and morphology are significantly influenced by the solvent type used; with increased temperature being tested the effect of solvent volatility, simplistically highlighted via boiling point and vapor pressure, as well as polymer-solvent interaction influenced by the solvent surface tension and dipole moment and dielectric constant will be of interest, for theoretically assessing any specific trends with solvent type and polymer with systematic testing of the ambient conditions. The physical properties of the solvents used are detailed in table 1 with a focus on properties that are influential within the ES technique. One would predict that with the use of chlorobenzene (PhCl) and deionised water, spherical particles with smooth surfaces would be ES due to the higher boiling points. Whereas with solvents such as chloroform and TFE due to high vapor pressure observed in the table, there is a higher likelihood of porous particles with increased temperature. With increased humidity and temperature, ES particles within the TFE system may be more likely to possess a higher level of polydispersity due to the high dipole moment, dielectric constant and lower surface tension, causing increased conductivity to the polymer and therefore reduced chain entanglement within the solution at increased ambient conditions.

| | Boiling | Vapour | Dipole | Dielectric | Surface |
|---|---|---|---|---|---|
| | Point | Pressure | Moment | constant | Tension |
| Solvent | (° C.) | (atm) at 20° C. | (D) | (εᵣ) | (dyne/cm) |
| DW | 100 | 0.023 | 1.85 | 80.1 | 72.8 |
| EtOH | 78 | 0.059 | 1.69 | 24.3 | 22.1 |
| CHCl₃ | 61 | 0.209 | 1.04 | 4.81 | 27.1 |
| PhCl | 131 | 0.012 | 1.54 | 5.69 | 33.28 |
| TFE | 78 | 0.102 | 2.52 | 8.55 | 16.5 |

PVP is a water and polar soluble polyamide, commonly used within the pharmaceutical field. For the ES of PVP, when ES spherical particles 1 μm in size are observed. However, with variation of temperature and humidity, ES PVP was significantly altered in each system.

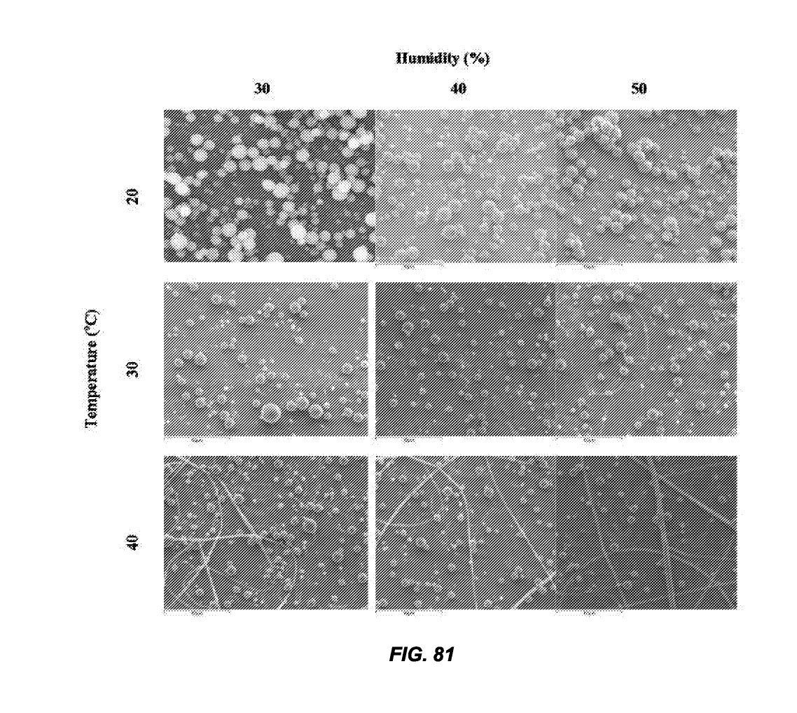

When DW was used as the solvent system to ES PVP the SEM images showed generally spherical particles with a smooth surface morphology (FIG. 81). However, at the extremities of temperature and humidity, with the exception in the 20° C. humidity range, the presence of fibers were present, most prevalent at 40° C. and 30% humidity. The dominant presence of fibers at 40° C. for all humidity's, indicate that the temperature within this set is more significant to the ES process than humidity. ES particles are spherical and smooth for all ambient conditions tested, with particles on average 1 μm in size. The particle size distribution is seen to be similar for all temperatures and humidity's (Table 2) with a minimum particle size of 0.3 μm and maximum particle size of 3.5 μm within the samples. All particles ES are generally 0.6 to1.8 μm in size. With most frequent particle size range increasing with increased humidity at 20° C. and 30° C., at 40° C. most particles are typically the same size; 0.8 to 1.2 μm.

The use of deionised water as the solvent within the polymer system, has provided a stability in average particle size within the system (Table 2) for all the temperatures and humidity's. However, though particle morphology has been significantly impacted through increasing temperature and humidity. As mentioned previously, deionised water has a high boiling point and low vapor pressure of 100° C. and 0.023 atm (Table 1), the polymer particles show a smooth surface morphology for all ambient conditions tested due to these solvent properties. With increased temperature, the polymer viscosity at the emitter tip is increased, due to more water evaporating at 40° C. As PVP in DW was already at a high polymer concentration, 50% (w/v) initially, there is already a high concentration of PVP particles within the solution and thus an increase in viscosity suggests increased polymer chain entanglement. As electrospinning and electrospraying use the same electrohydrodynamic process, one of the main variables that influence the process that occurs is polymer concentration. Thus, when the temperature is set to 40° C., enough water evaporates at the emitter tip to cause an increased polymer concentration and thus the electrospraying process to be altered to electrospinning. The presence of increased humidity on the other hand for polymer morphology, influences the polydispersity of the particles as observed within the 20° C. range and noted in table 2. Increased humidity causes a higher influx of water to diffuse into the polymer solution during the electrospraying process. As PVP is already water soluble, it does not undergo a phase separation as extensively as a hydrophobic polymer would and therefore the is a non-uniformity in the rate of diffusion within the atomised particles during the electrospray.

FIG. 81 illustrates SEM images of ES PVP in deionised water within a climate control chamber at temperature sets 20° C., 30° C. and 40° C. with humidity at each temperature set tested at 30, 40% and 50%.

Table 2 includes ES PVP in DW particle size ranges at 20, 30 and 40° C. for 30,40 and 50% humidity.

| | | Average | | |
|---|---|---|---|---|
| | | Particle | | Highest |
| | | Size ± | | frequency of |
| Temperature | | Standard | Particle size | particle size |
| (° C.) | Humidity (%) | Error (μm) | range (μm) | (μm) |
| 20 | 30 | 1.49 ± 0.04 | 0.5-2.8 | 1.5-1.8 |
| | 40 | 1.34 ± 0.04 | 0.5-2.4 | 1.1-1.5 |
| | 50 | 1.26 ± 0.04 | 0.3-2.7 | 1-1.4 |
| 30 | 30 | 1.13 ± 0.04 | 0.3-2.2 | 0.6-0.9 |
| | | | 2.8-3.1 | |
| | 40 | 0.95 ± 0.04 | 0.3-1.9 | 0.8-1.1 |
| | 50 | 1.2 ± 0.03 | 0.3-2.2 | 1.1-1.4 |
| | | | 3.2-3.5 | |
| 40 | 30 | 1.1 ± 0.05 | 0.5-3 | 0.8-1.2 |
| | 40 | 0.99 ± 0.02 | 0.5-1.7 | 0.8-1 |
| | 50 | 0.98 ± 0.02 | 0.4-1.7 | 0.9-1.1 |

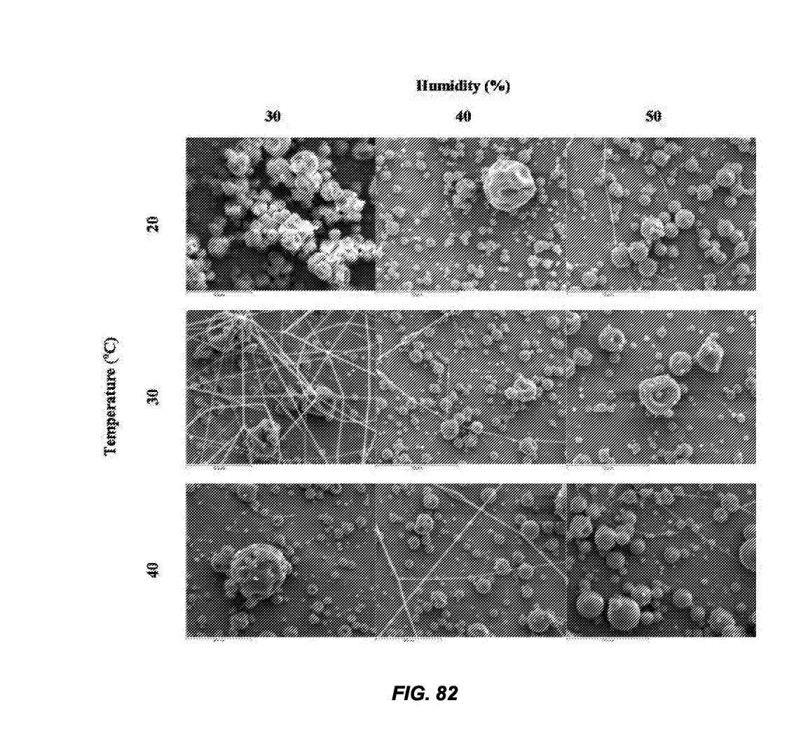

When PVP was ES in an EtOH system at the same temperature and humidity ranges, the results showed a significant impact of the ambient conditions on the ES PVP morphology (FIG. 82). The particles ES showed a spherical appearance, however the surface of the PVP particles is predominantly indented. The surface morphology is shown to be altered from indented to smooth, through the presence of increased humidity at all three temperature sets.

The average particle size is similar at all temperatures and humidity's (table 3) between 1-2 μm, with particle distribution quite large, a minimum size of 0.3 μm and a maximum size of 8.7 μm. At 20° C., the humidity range does not significantly alter the ES process with PVP particle formation maintained at each humidity. At 50% humidity, small fibers, 300 nm in size appear within the samples. It could be assumed that with further increased humidity, there would be increased fiber presence and a conversion to electrospinning rather than electrospraying. The surface morphology of the particles shows smooth surfaces for smaller particles and indented surfaces on the larger particles. At 30° C., the PVP electrohydrodynamic process undergoes an opposing trend to 20° C., whereby with increased humidity there is a reduction in the formation of PVP fibers with particles. At 30% humidity, the most fibers are present, typically 200 to 300 nm in size. At 50% humidity, the particles show two distinct surface morphologies; a smoother surface morphology on the smaller particles and an indented ‘doughnut’ shaped morphology on the larger particles. At 40° C., the trend with humidity is unclear, there is a significant difference in morphology at each humidity. At 30% humidity PVP ES is maintained, with only particles observed within the sample. The surface morphology of the particles shows a mixture of surfaces present; smooth surfaces, ‘doughnut’ shapes as well as indented. At 40% humidity, there is a presence of PVP fibers with particles within the sample. The fibers are 200 to 400 nm in size. The surface morphology of the particles shows a mainly smooth surface on most particles, with a few larger particles with indented surface morphologies. At 50% humidity, sparse tiny fibers of PVP 100 to 200 nm in size with smooth, spherical PVP particles are present.

Within the EtOH system, humidity is shown to have a significant impact on the secondary surface morphology of the ES particles. At lower humidity's, for each temperature due to the solvent properties of EtOH, lower boiling point, higher vapor pressure and lower surface tension (table 2), 78° C., 0.059 atm and 22.1 dyne/cm compared with deionised water. The breath figure process can be seen as occurring. As EtOH is evaporating at a high rate from the polymer solution when it is at the tip, with water vapor present at 20 and 30% humidity predominately a certain amount of water has condensed onto the surface of the particles, causing non-uniform evaporation of EtOH from the ES particle and thus the indented morphology. With increased humidity the predominant process that is influencing the secondary surface morphology of the PVP ES particles is vapor phase separation; whereby the addition water vapor diffuses at a higher gradient into the PVP atomised particles during ES and causes an increase in the surface tension of the PVP particle. Thus causing the surface to have a more rigid structure and larger ability for remaining EtOH to evaporate at a more uniform rate over the whole surface. The particle size for PVP in EtOH as with DW is similar in size, as PVP in the EtOH system was ES at a lower concentration, 25% (w/v) PVP compared to 50% (w/v) for DW, one could highlight that due to the higher conductivity of EtOH compared to water, less polymer is required for the ES process; with efficient amount of charge present to the PVP particles within the solution at the emitter tip. This is supported by the presence of large fibers at 30° C. at the lower humidity. The temperature and humidity provide a threshold for highest rate of solvent evaporation and lack of water vapor diffusion for altering the polymer viscosity to a critical concentration where the ES process is altered to electrospinning. The particles present at 30° C. show an irregularity in solvent evaporation and also thermally induced phase separation (TIPS). Whereby the solvent is evaporating during the ES process, at a faster rate at the outer edges than within the core. Due to this fast solvent evaporation the temperature exhibiting on the particles is significantly altered. Causing the inner core to shrink at a higher rate than the outer part of the particle; to conserve a thermal equilibrium. Thus causing the formation of cup-like particle morphologies.

With increased temperature 40° C., the rate of solvent evaporation reaches an equilibrium for the inner and outer part of the PVP particles during ES, as well as a higher diffusion gradient for water vapor to enter the particles, causing the smoothened, spherical appearance.

FIG. 82 illustrates SEM images of ES PVP in EtOH within a climate control chamber at temperature sets 20° C., 30° C. and 40° C. with humidity at each temperature set tested at 30, 40% and 50%.

Table 3 shows ES PVP in EtOH particle size ranges at 20, 30 and 40° C. for 30,40 and 50% humidity.

| | | Average | | |
|---|---|---|---|---|
| | | Particle | | Highest |
| | | Size ± | | frequency of |
| Temperature | | Standard | Particle size | particle size |
| (° C.) | Humidity (%) | Error (μm) | range (μm) | (μm) |
| 20 | 30 | 2.12 ± 0.11 | 0.4-5 | 1.9-2.7 |
| | 40 | 0.98 ± 0.04 | 0.3-2.1 | 0.9-1.2 |
| | | | 6.1-6.5 | |
| | 50 | 1.27 ± 0.05 | 0.3-3 | 1-1.4 |
| | | | 3.3-3.7 | |
| 30 | 30 | 1.33 ± 0.06 | 0.4-3.4 | 1.4-1.9 |
| | | | 4.9-6 | |
| | 40 | 1.48 ± 0.1 | 0.3-6.3 | 0.3-1 |
| | 50 | 1.35 ± 0.06 | 0.3-5 | 0.7-1.2 |
| 40 | 30 | 1.58 ± 0.06 | 0.3-4.9 | 1-1.8 |
| | | | 7.9-8.7 | |
| | 40 | 1.36 ± 0.08 | 0.2-5.7 | 0.8-1.5 |
| | 50 | 1.46 ± 0.11 | 0.3-4.5 | 0.3-1.2 |
| | | | 5.4-7.9 | |

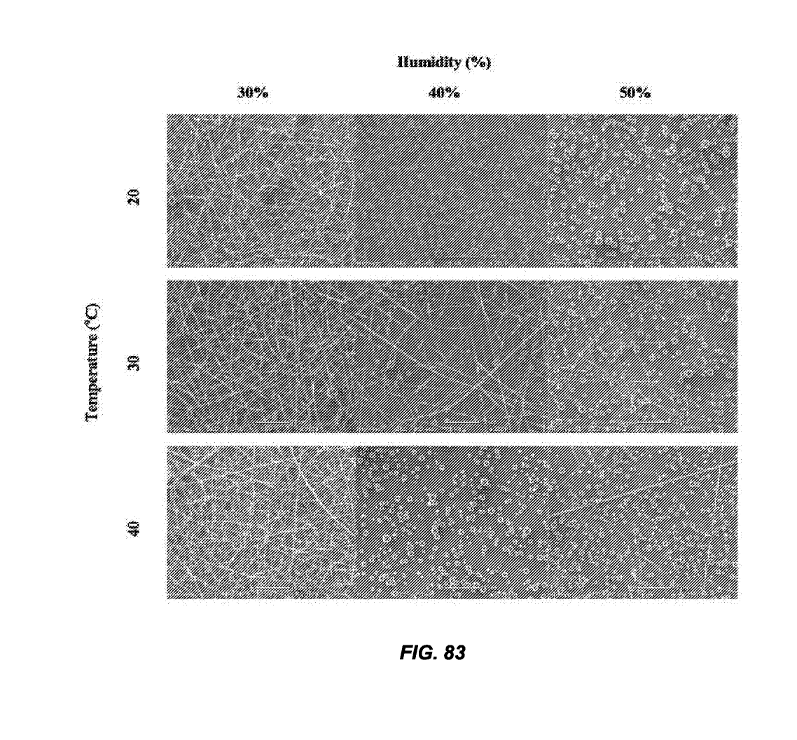

PVP in a 1:1 ratio of DW and EtOH (v/v) within the preliminary experiments showed electrospraying characteristics only at a narrow concentration; 12.5% (w/v) with lower concentrations at 20° C., 30% RH showing excessive dripping during the process and higher temperatures showing prominent electrospinning characteristics. As multiple samples were taken for each test; at 1.5% (w/v) ES characteristics and the presence of particles were observed, but mainly electrospun bead's on a string were seen, thus the representation of beads-on-a-string in FIG. 83. The results show that particle when ES PVP particles were present were still on average 1 μm in size (table. 4), as well as the significance of the ambient conditions on the alteration of the electrohydrodynamic process, as the predominately electrospinning process was altered to electrospraying with increased humidity.

In the 20° C. set experiments, PVP is shown to have a ‘beads on string morphology’ at 30% humidity. The average particle size on the fibers is 1.1 μm with fibers 300 to 400 nm in size. The particle surfaces and fibers show a smooth surface morphology within the system. At 40% humidity, the smooth surface morphology is maintained, with smaller fibers present within the ‘beads on string’ morphology. Particles are on average 1.2 μm in size with fibers typically 200 to 300 nm in size. At 50% humidity, only spherical particles with smooth surfaces are present within the system, average particle size of 1.3 μm measured. The particle size range at these ambient conditions is between 0.2 to 2.5 μm with most particles 0.6 to 1 μm in size. Increasing the temperature range to 30° C., 30% humidity, fibers are predominately present with sparse particles present within the sample. The fiber size is on average 0.3 μm in size with smooth surface morphologies. At 40% humidity, there is an increased presence of smooth particles within a ‘beads on string’ morphology, with particles on average 1.1 μm in size and fibers between 100 to 600 nm. At 50% humidity, the particles have a more spherical, smooth appearance with the sample predominately comprised of particles on average 1.1 μm in size with fibers between 180 to 350 nm.

At 40° C. 30% humidity, there is a mixture of smooth fibers and ‘beads on strings’ within the sample. With the beads on average lgm in size, whereas the fibers are on average 242 nm in size and between 70 to 440 nm. At 40% humidity, the samples showed mainly particles present, with smooth, spherical morphologies, the average particle size was 1.2 μm with a particle range between 0.4 to 2.4 μm with most particles 1 to 1.2 μm in size. At 50% humidity, there is an increasing presence of smooth fibers, however most the sample is comprised of smooth spherical PVP particles. The average particle size is 1.1 μm with to particle size ranges, many the particles were between 0.3 to 1.9 μm with most particles 0.9 to 1.3 μm in size and there were a few larger particles present within the sample 2.5 to 2.9 μm in size. The fibers on the other hand were on average 239 nm with a fiber size range 95 to 855 nm.

The presence of smooth surfaces for both the fibers and particles at all ambient conditions tested, show the prevalence of DW within the system, when compared PVP within the individual solvent systems discussed previously. Whereas the preference of electrospinning to electrospraying within the ambient conditions appears to follow the same pattern observed as with PVP within the EtOH system. However, the mixed solvent system with PVP, causes the presence of fibers and bead on a string electrospinning morphologies at a higher rate than within the individual system. Ambient conditions are shown significantly affect the process however, as with increased humidity electrospinning is altered to electrospraying. The use of higher temperatures, improve the alteration between electrospinning to electrospraying at lower humidity, for example at 20° C., 50% provides ES of spherical, smooth particles whereas at 40° C., 40% humidity, spherical, smooth particles are present.

One could theorize that the PVP in the 1:1 DW:EtOH system has a high conductivity due to the low PVP concentration required, 12.5% (w/v), and increased charge and chain entanglement generated to cause the alignment of the polymer particles for stretching into fibers rather than atomised into particles. Increased water vapor present with higher humidity would in theory be diluting the polymer solution at the tip, to provide a decreased chain entanglement and viscosity for atomisation to occur. With increased temperature, EtOH within the system evaporates at a faster rate, providing a higher diffusion gradient for water vapor to enter the solution at the emitter and cause the solution to gain more ES properties.

FIG. 83 illustrates SEM images of ES PVP in deionised water and EtOH at a 1:1 (v/v) ratio, within a climate control chamber at temperature sets 20° C., 30° C. and 40° C. with humidity at each temperature set tested at 30, 40% and 50%.

Table 4 illustrates ES PVP in EtOH and H₂O showing particle ranges at 20,30 and 40° C. for 30,40 and 50% humidity.

| | | Average | | |
|---|---|---|---|---|
| | | Particle | | Highest |
| | | Size ± | | frequency of |
| Temperature | | Standard | Particle size | particle size |
| (° C.) | Humidity (%) | Error (μm) | range (μm) | (μm) |
| 20 | 30 | 1.13 ± 0.04 | 0.65-1.9 | 0.9-1.2 |
| | 40 | 1.21 ± 0.06 | 0.46-2.8 | 0.85-1.24 |
| | 50 | 1.31 ± 0.05 | 0.24-2.83 | 0.61-0.98 |
| 30 | 30 | — | — | — |
| | 40 | 1.12 ± 0.04 | 0.33-1.89 | 0.85-1.11 |
| | 50 | 1.13 ± 0.04 | 0.36-2.28 | 1-1.32 |
| 40 | 30 | 1.01 ± 0.05 | 0.56-2.11 | 0.87-1.18 |
| | 40 | 1.16 ± 0.04 | 0.4-2.36 | 0.96-1.24 |
| | 50 | 1.11 ± 0.04 | 0.31-2.87 | 0.95-1.27 |

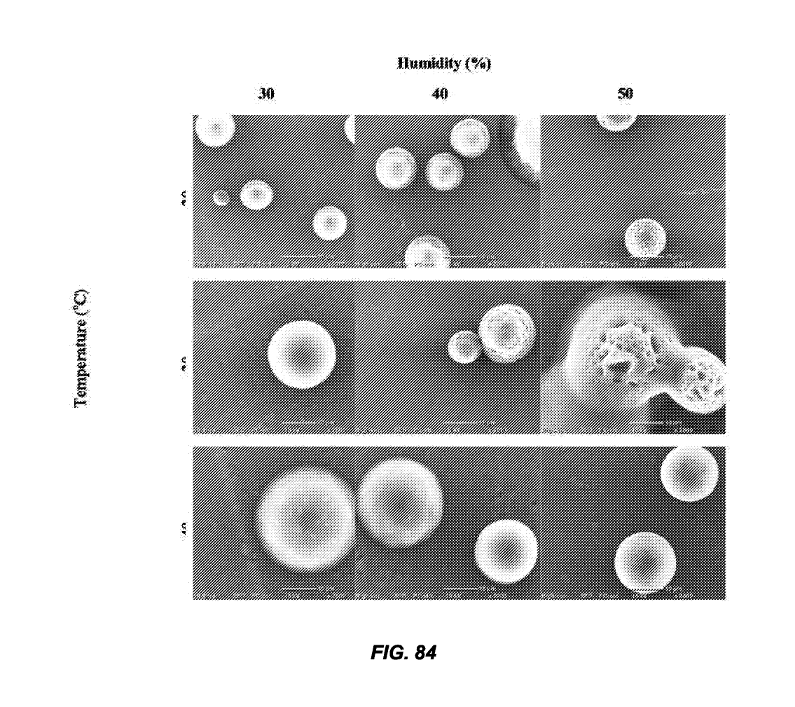

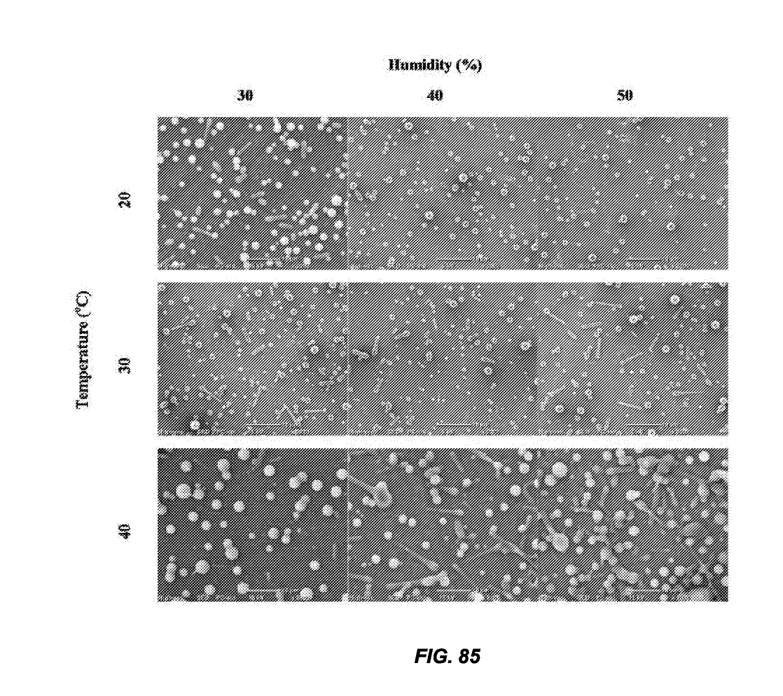

The use of alternate polymers was required for investigations on the behavior of inorganic solvent systems in relation to ambient conditions. PCL was chosen, as it is an FDA approved polymer and is used within drug delivery applications. The PCL solution was electrosprayed within the climatic control chamber and the results showed (FIG. 84) that temperature and humidity did alter the size and surface morphology of the particles. At all temperature ranges and humidity's PCL maintained ES and thus the fabrication of particles. The surface morphology however did alter with increasing humidity. The particles changed from smooth surfaces at low humidity, 30%, to porous surfaces at higher humidity, 50%. However, porosity was not observed for the 40° C. humidity range, instead particle integrity was shown to decrease with increasing temperature, with particle size significantly becoming larger (Table 5) and particles showing the presence of primary and satellite particles (FIG. 85).

In the 20° C. range, the particles showed smooth surface spherical particles at 30%, with an average particle size of 10 μm and particles within two particle size ranges; a small number of particles 4-6 μm and most particles 8 to 13 μm. With increasing humidity to 40%, the particles become larger with an average particle size of 13 μm and a large distribution of particle sizes ranging from 8 to 23 μm, with most particles 10 to 16 μm in size. The surface morphology of the spherical particles was still smooth. This surface morphology was altered at 50% humidity, with a porous and roughened surface morphology observed on the spherical particles. The average particle size was 14 μm and the particle size range between 8 to 25 μm.

At 30° C. a similar trend was observed, the particles at 30% humidity were spherical with smooth surface morphology and average particle size of 19 μm, with a particle size range between 12 to 27 μm, with most particles 15 to 19 μm in size. At 40% humidity, the spherical particles had the presence of pores on the surface, with a decreased average particle size of 12 μm and two particle size ranges, a smaller particle size range between 7 to 19 μm with most particles 10 to 13 μm in size and a larger particle size range between 22 to 25 μm. At 50% humidity, the particles have lost the spherical shape and instead have fused polymer particles with a highly porous structure. The average particle size is 24 μm and particle size range is very large, between 9 to 39 μm.

At 40° C., the integrity of the spherical particles appears to be less solid when ES, the resulting particles still show a smooth surface morphology with the average size the largest seen with the PCL particles at 29 μm, with a particle size range of 15 to 40 μm, with most particles 23 to 32 μm in size. With increasing humidity, 40%, the particles still show a spherical shape and smooth surface morphology. The average particle size has decreased to 24 μm and the particle size range is 14 to 37 μm, with most particles 20 to 25 μm in size. At 50% the particles still show a spherical, smooth surface morphology with average particle size 19 μm with size range between 14 to 32 μm with most particles 14 to 19 μm in size.

The two solvents used within the polymer system were chlorobenzene and chloroform, both have significantly different boiling points and vapor pressure values (table 1). With Chlorobenzene having a higher boiling point of 131° C., the highest of the solvents tested, and a vapor pressure of 0.012 atm. Whereas chloroform has a boiling point of 61° C. and a vapor pressure of 0.209 atm, the solvent system comprised of an 8:2 ratio of chlorobenzene to chloroform (v/v). As for all the ambient conditions the electrohydrodynamic process was maintained as electrospraying, when compared with the previous systems, the significantly lower dielectric constants of this solvent system compared with EtOH and DW, provide less charge to the polymer molecules within the solution for a higher degree of entanglement to occur.

The higher boiling point of chlorobenzene which is the main component of the solvent system, follows a similar trend to DW, whereby at lower humidity the particle surface morphology is smooth due to a slower rate of solvent evaporation from the polymer. The presence of a roughened surface at higher humidity, but not a porous structure at 20° C., 50%, highlight that the influx of water vapor within the particles during ES, are forming a skin layer, due to the hydrophobic nature of the polymer. However due to the low rate of solvent evaporation occurring at this temperature, the polymer is not undergoing breath-figure translations. With the increased temperature however, significant pores form on the surface of the particles.

This can be due to the accelerated solvent evaporation of chloroform and so breath figure translation can occur with a higher diffusion gradient for water vapor to influx onto the polymer surface, causing cracks in the polymer particles. At 40° C., the porous nature of the PCL particles are not present, even with increased humidity. The polymer at lower humidity is seen to have a more conjoined, less solid structure, which could be due to the particles unable to fully solidify before reaching the collector as the solvent has evaporated at such a fast rate, that the polymer integrity has come into question. With increased humidity, the polymer particles have a spherical smooth structure; therefore the influx of water vapor within the surface induces enough surface tension within the particles to maintain the integrity of the particles.

FIG. 84 illustrates SEM images of ES PCL in Chloroform: Chlorobenzene at a 80:20 (v/v) ratio, within a climate control chamber at temperature sets 20° C., 30° C. and 40° C. with humidity at each temperature set tested at 30, 40% and 50%.

Table 5 illustrates ES PCL in CHCL3:CHb showing particle size ranges at 20,30 and 40oC for 30,40 and 50% humidity.

| | | Average | | Highest |
|---|---|---|---|---|
| | | Particle Size ± | | frequency of |
| Temperature | Humidity | Standard | Particle size | particle size |
| (° C.) | (%) | Error (μm) | range (μm) | (μm) |
| 20 | 30 | 9.97 ± 0.27 | 4.63-13.13 | 9.73-11.43 |
| | 40 | 13.35 ± 0.45 | 8.03-22.53 | 10.93-16.73 |
| | 50 | 14.57 ± 0.87 | 8.09-25.29 | 12.39-16.69 |
| 30 | 30 | 19.11 ± 0.59 | 11.55-26.75 | 15.35-19.15 |
| | 40 | 11.93 ± 0.45 | 7.36-25.36 | 10.36-13.36 |
| | 50 | 23.52 ± 2.85 | 9.33-39.23 | 9.33-19.33 |
| 40 | 30 | 28.66 ± 1.53 | 15.41-40.31 | 23.71-32.01 |
| | 40 | 23.53 ± 0.92 | 14.1-36.5 | 19.7-25.3 |
| | 50 | 19.79 ± 0.71 | 14-32 | 14-18.5 |

To investigate the effect of inorganic solvents with ambient temperatures, an additional polymer was tested. PLGA is also an FDA approved polymer that is used within drug delivery. It was dissolved in TFE and showed differing trends to that observed with PCL in chloroform and chlorobenzene. Ambient conditions were shown to affect PLGA ES (FIG. 85), with temperature affecting more significantly the size of the particles, whereas humidity affecting the shape. As with PCL, the inorganic solvent system prevented the production of fibers with altered temperature and humidity.

At 20° C., humidity affects the shape of the particles produced, with increased humidity improving the uniformity of the particle size and shape. At 30% humidity, there was a mixture of spherical smooth particles and elongated cylindrical smooth particles present. The average particle size was 1.9 μm with the particle range between 0.5 to 3.4 μm with most of the particles 1.6 to 1.9 μm. At 40% humidity, most of the ES PLGA shows spherical, smooth particles, with sparse elongated cylindrical PLGA shapes present within the sample. The average particle size was 1.3 μm and the particle range was 0.4 to 2.2 μm with most of the particles 1.1 to 1.4 μm in size. At 50% humidity, the particles are all spherical and smooth, with an average particle size of 1.3 μm with particles between 0.5 to 2.5 μm and most particles 0.9 to 1.3 μm in size.

At 30° C., 30% humidity, the particles within the samples showed mixed morphologies of spherical, cylindrical and spherical particles with long triangular tails attached. All the surface morphologies were smooth and the average particle size as 1.2 μm. The particle sizes calculated showed two different ranges; one where most particles were between 0.3 to 2.2 μm, with most particles 0.9 to 1.2 μm and a few particles 2.5 to 2.8 μm in size. At 40% humidity, the particle morphology still shows a mixed shape morphology, from spherical, cylindrical and spherical particles but with less elongated tails. The average particle size is 1.2 μm with particles within the range of 0.4 to 2.8 μm and most particles typically 0.8 to 1.2 μm in size. At 50% humidity, the morphology of the particles still showed a mix of cylindrical, spherical and spherical particles with the most elongated tails seen within the humidity rage at 30° C. The average particle size was 1.2 μm with most particles as seen with all humidity's within the range between 0.4 to 2.8 μm and most particles 0.8 to 1.2 μm.

At 40° C., 30% humidity, particles are seen to be mostly spherical in shape with smooth surfaces. The average particle size is 2.9 μm with particles within three size ranges; 1.2 to 4.5 μm, with most particles 2.3 to 3.4 μm, and larger particles with sizes between 6.7 to 7.8 μm and 8.9 to 10 μm. The large size distribution, although showing the prevalence of spherical particles, does not provide a uniformity in size. At 40% humidity, the particles show a loss of integrity in particle shape, with spherical particles present as well as molded shapes of spherical and cylindrical. The average particle size was 2.6 μm with particles 1.4 to 4.7 μm in size and most particles deposited 2.5 to 3 μm. At 50% humidity, the loss of integrity in particle shape is still present, with spherical particles present as well as molded spherical and cylindrical shapes. The average particle size is 2.6 μm, with particles between 1.3 to 4 μm deposited and most particles 2.4 t 2.9 μm.

TFE has the same boiling point as EtOH, 78° C., but a higher vapor pressure. This helps to support the theory that the increased rate of solvent evaporation from the particles during the ES process cause alteration in the morphology with a more indented or porous structure, such as for PVP in EtOH. The dipole moment for TFE is the highest of all the solvents at 2.52 D (Table 1) with the lowest surface tension, 16.5 dyne/cm. These two solvent properties support the alteration in surface morphology and increased polydispersity, with spherical particles and cylindrical particles, with the higher temperatures increasing the elongation observed. It has been observed that this alteration in morphology, with the elongated structures attributed to initial coloumb fission and then a fast rate of solvent evaporation that caused the droplets to freeze within an irregular shape, due to alterations in the temperature gradient exhibited onto the PLGA particles during the ES process. As with increased temperature, solvent evaporation would be more prominent, the elongation of the particles can be seen, as shown in the 30° C. set. Due to the lower surface tension, the particles are able to break the compact shape of the atomised primary particle to enhance the alteration in surface morphology. One could assume, as shown in the 20° C. set, that the increased amount of water vapor present with increased humidity, causes an influx of water within the system that reduces the polarity of the polymer particles and reduces the temperature gradient observed within the polymer particle through solvent evaporation. Water has a higher surface tension and vapor pressure and so the adsorption onto the polymer particle surfaces can be seen to cause a contraction in the primary particle shape. This trend however is not observed with the higher temperatures, the TFE rate of solvent evaporation would be presumed as being too fast for the influx of water adsorption to correct the thermodynamic instabilities and thus the polymer's surface tension is not increased, causing increased elongation and in the case at 40° C., the solvent evaporation causes the polymer to not solidify effectively during the ES process and so have a wet morphology.

FIG. 85 illustrates SEM images of ES PLGA in TFE, within a climate control chamber at temperature sets 20° C., 30° C. and 40° C. with humidity at each temperature set tested at 30, 40% and 50%.

Table 6 illustrates ES PLGA in TFE showing particle size ranges at 20, 30 and 40° C. for 30,40 and 50% humidity.

| | | Average | | |
|---|---|---|---|---|
| | | Particle | | Highest |
| | | Size ± | | frequency of |
| Temperature | | Standard | Particle size | particle size |
| (° C.) | Humidity (%) | Error (μm) | range (μm) | (μm) |
| 20 | 30 | 1.92 ± 0.05 | 0.48-3.36 | 1.56-1.92 |
| | 40 | 1.25 ± 0.05 | 0.35-2.51 | 1.07-1.43 |
| | 50 | 1.31 ± 0.06 | 0.46-2.46 | 0.86-1.26 |
| 30 | 30 | 1.23 ± 0.04 | 0.31-2.79 | 0.93-1.24 |
| | 40 | 1.23 ± 0.06 | 0.37-2.83 | 0.78-1.19 |
| | 50 | 1.29 ± 0.06 | 0.42-2.82 | 0.82-1.22 |
| 40 | 30 | 2.94 ± 0.16 | 1.23-10.03 | 2.33-3.42 |
| | 40 | 2.65 ± 0.08 | 1.39-4.69 | 2.49-3.04 |
| | 50 | 2.64 ± 0.08 | 1.26-4.06 | 2.38-2.94 |

### REFERENCES

1. Syandan Chakraborty, I-Chien Liao, Andrew Adler, Kam W. Leong (2009) Electrohydrodynamics: A facile technique to fabricate drug delivery systems. Advanced Drug Delivery Reviews 61, 1043-1054, doi:10.1016/j.addr.2009.07.013
2. Radhakrishnan Sridhar & Seeram Ramakrishna (2013) Electrosprayed nanoparticles for drug delivery and pharmaceutical applications, Biomatter, 3:3, e24281, DOI:10.4161/biom.24281
3. Jyothi Sri, S., Seethadevi, A., Suria Prabha, K., Muthuprasanna, P., & Pavitra, P. (2012). Microencapsulation: A review. International Journal of Pharma and Bio Sciences, 3(1), P509-P531. http://doi.org/10.1007/BF00569928
4. Ana Leticia Braz and Ifty Ahmed (2017) Manufacturing processes for polymeric micro and nanoparticles and their biomedical applications. AIMS Bioengineering, 4(1): 46-72. DOI: 10.3934/bioeng.2017.1.46
5. Oliver Hardick, Bob Stevens, Daniel G. Bracewell (2011) Nanofibre fabrication in a Temperature and humidity controlled environment for improved fibre consistency. J Mater Sci (2011) 46:3890-3898. DOI 10.1007/s10853-011-5310-5
6. Jianfen Zheng, Haiyuan Zhang, Zhiguo Zhao, Charles C. Han (2012) Construction of hierarchical structures by electrospinning or electrospraying Polymer 53, 546-554, doi:10.1016/j.polymer.2011.12.018
7. Jianfen Zheng, Aihua He, Junxing Li, Jian Xu, Charles C. Han (2006) Studies on the controlled morphology and wettability of polystyrene surfaces by electrospinning or electrospraying. Polymer 47, 7095-7102, doi:10.1016/j.polymer.2006.08.019
8. Masashi Ikeuchi, Ryosuke Tane & Koji Ikuta (2012) Electrospray deposition and direct patterning of polylactic acid nanofibrous microcapsules for tissue engineering. Biomed Microdevices, 14:35-43, DOI 10.1007/s10544-011-9583-x
9. Taehoon Kim, Seung Jae Yang, Sae Jin Sung, Yern Seung Kim, Mi Se Chang, Haesol Jung and Chong Rae Park (2015) Highly Reproducible Thermocontrolled Electrospun Fiber Based Organic Photovoltaic Devices. ACS Appl. Mater. Interfaces 2015, 7, 4481-4487, DOI: 10.1021/am508250q
10. S. De Vrieze, T. Van Camp, A. Nelvig, B. Hagström, P. Westbroek, K. De Clerck (2009) The effect of temperature and humidity on electrospinning. J Mater Sci, 44:1357-1362, DOI 10.1007/s10853-008-3010-6
11. Jan Pelipenko, Julijana Kristl, Biljana Jankovi′c, Sa\check{ }sa Baumgartner, Petra Kocbek (2013) The impact of relative humidity during electrospinning on the morphology and mechanical properties of nanofibers. International Journal of Pharmaceutics 456, 125-134
12. Chi Hun Kim, Yoon Ho Jung, Hak Yong Kim, and Douk Rae Lee (2006) Effect of Collector Temperature on the Porous Structure of Electrospun Fibers. Macromolecular Research, Vol. 14, No. 1, pp 59-65
13. Deng-Guang Yu, Xia-Xia Shen, Chris Branford-White, Kenneth White, Li-Min Zhul and S W Annie Bligh (2009) Oral fast-dissolving drug delivery membranes prepared from electrospun polyvinylpyrrolidone ultrafine fibers. Nanotechnology, Volume 20, Number 5.
14. Begoña Almería, Weiwei Deng, Tarek M. Fahmy, Alessandro Gomez (2010) Controlling the morphology of electrospray-generated PLGA microparticles for drug delivery. Journal of Colloid and Interface Science 343, 125-133
15. Fanzheng Meng, Yi Jiang, Zhihui Sun, Yizi Yin, Yanyan Li (2009) Electrohydrodynamic Liquid Atomization of Biodegradable Polymer Microparticles: Effect of Electrohydrodynamic Liquid Atomization Variables on Microparticles. Journal of Applied Polymer Science; DOI 10.1002
16. Cheryl L. Casper, Jean S. Stephens, Nancy G. Tassi, D. Bruce Chase and John F. Rabolt (2004) Controlling Surface Morphology of Electrospun Polystyrene Fibers: Effect of Humidity and Molecular Weight in the Electrospinning Process. Macromolecules, 37, 573-578
17. Silke Megelski, Jean S. Stephens, D. Bruce Chase and John F. Rabolt (2002) Micro- and Nanostructured Surface Morphology on Electrospun Polymer Fibers. Macromolecules, 35, 8456-8466
18. Gökçe Yazgan, Ruslan I. Dmitriev, Vasundhara Tyagi, James Jenkins, Gelu-Marius Rotaru, Markus Rottmar, René M. Rossi, Claudio Toncelli, Dmitri B. Papkovsky, Katharina Maniura-Weber2 & Giuseppino Fortunato (2017) Steering surface topographies of electrospun fibers: understanding the mechanisms. Scientific Reports, 7: 158, DOI:10.1038/s41598-017-00181-0
19. Liwei Huang, Nhu-Ngoc Bui, Seetha S. Manickam, Jeffrey R. McCutcheon (2011) Controlling Electrospun Nanofiber Morphology and Mechanical Properties Using Humidity. journal of polymer science part b: polymer physics, 49, 1734-1744
20. Yong Liu, 1 Ji-Huan He, Jian-yong Yu and Hong-mei Zeng (2008) Controlling numbers and sizes of beads in electrospun nanofibers. Polym Int 57:632-636
21. Ping Lu and Younan Xia (2013) Maneuvering the Internal Porosity and Surface Morphology of Electrospun Polystyrene Yarns by Controlling the Solvent and Relative Humidity. Langmuir 2013, 29, 7070-7078
22. Chia-Ling Pai, Mary C. Boyce and Gregory C. Rutledge (2009) Morphology of Porous and Wrinkled Fibers of Polystyrene Electrospun from Dimethylformamide. Macromolecules, 42, 2102-2114
23. Milad Jafari-Nodoushana, Jalal Barzinb and Hamid Mobedia (2015) Size and morphology controlling of PLGA microparticles produced by electrohydrodynamic atomization. Polym. Adv. Technol., 26 502-513
24. Ivo B. Rietveld, K. Kobayashi, H. Yamada, K. Matsushige (2006) Morphology control of Poly(vinylidene fluoride) thin film made with electrospray. Journal of Colloid and Interface Science 298, 639-651
25. Quanchao Zhang, Jing Liu, Xiaojun Wang, Minxiong Li & Jie Yang (2010) Controlling internal nanostructures of porous microspheres prepared via electrospraying. Colloid Polym Sci, 288:1385-1391
26. Jianfen Zheng, Aihua He, Junxing Li, Jian Xu, Charles C. Han (2006) Studies on the controlled morphology and wettability of polystyrene surfaces by electrospinning or electrospraying. Polymer 47, 7095-7102 Jianfen Zheng, Haiyuan Zhang, Zhiguo Zhao, Charles C. Han (2012) Construction of hierarchical structures by electrospinning or electrospraying. Polymer 53, 546-554

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. An apparatus comprising:
an electrospraying emitter;
a first current measuring unit electrically coupled to the emitter and measuring an emitter current;
a counter-electrode;
a second current measuring unit electrically coupled to the counter-electrode and measuring a counter-electrode current; and
a controller configured to:
receive an emitter current measurement and a counter-electrode current measurement;
calculate, based on the received emitter current measurement and the received counter-electrode current measurement, a current adjustment value to compensate for parasitic current loss between the emitter and the counter-electrode; and
adjust the emitter current based on the calculated current adjustment value.

2. The apparatus of claim 1, further comprising:
a current source electrically coupled to the emitter, the current source providing current at a voltage greater or less than 500 Volts relative to the counter-electrode.

3. The apparatus of claim 1, further comprising:
an array of emitters including a first emitter and a second emitter, wherein the emitter is the first emitter; and
wherein the controller is configured to energize the first emitter for a first period of time and to energize the second emitter for a second period of time, wherein the first period of time and the second period of time are non-overlapping.

4. The apparatus of claim 1, further comprising:
a microfluidic solution source configured to provide solution continuously to the emitter.

5. The apparatus of claim 1, wherein the first current measuring unit is a high voltage nano-ammeter.

6. The apparatus of claim 1, wherein the emitter includes a cannula for dispersing fluid.

7. The apparatus of claim 1, wherein the counter-electrode is arranged to receive dispersed charged solution emitted by the emitter.

8. The apparatus of claim 1, wherein the counter-electrode includes gold, Indium-tin-oxide (ITO), copper, nickel-plated copper, or stainless steel.

9. The apparatus of claim 1, wherein the emitter disperses liquid into an environment having between 0.1 atmosphere and 10 atmosphere.

10. The apparatus of claim 1, further comprising a liquid source including a gravity reservoir.

11. The apparatus of claim 1, further comprising a liquid source including an electro-osmatic (EO) pump that has an electrical potential greater than the emitter.

12. The apparatus claim 1, further comprising:
an extractor arranged between the emitter and the counter-electrode, the extractor having an electric potential difference from the counter-electrode that is less than the electric potential difference between the emitter and the counter-electrode, the extractor including an adjustable annular aperture.

13. The apparatus of claim 1, wherein calculating a current adjustment value comprises:
subtracting the measured counter-electrode current from the measured emitter current.

14. The apparatus of claim 1, wherein the second current measuring unit is a current mirror.

15. The apparatus of claim 1, further comprising:
an emitter switch coupling the emitter to a power source and receiving a control signal;
wherein adjusting the emitter current based on the calculated current adjustment value includes modifying a duty cycle of the control signal, the control signal pulse width modulated.

16. The apparatus of claim 15, wherein the duty cycle is between 1 and 99 percent.

17. The apparatus of claim 16, wherein the duty cycle is about 10, 50, 70, or 90 percent, wherein about is within 10 percent.

18. The apparatus of claim 15, wherein the control signal includes a frequency between 1 Hertz and 10,000 Hertz.

19. The apparatus of claim 18, wherein the frequency is about 1, 100, or 1000 Hertz, wherein about is within 10 percent.

20. The apparatus of claim 1, further comprising a mixing element fluidically connected to the emitter, the mixing element for mixing polymer and cells prior to provision to the emitter for electro spraying.

21. The apparatus of claim 1, further comprising an image acquisition device arranged to view a region between the emitter and the counter electrode, the image acquisition device configured to acquire an image of the region;
wherein the controller is configured to, using the image of the region, detect a characteristic of a particle within the region.

22. The apparatus of claim 21, further comprising a rejection element operatively coupled to the controller, wherein the controller is further configured to determine that the detected characteristic does not satisfy a criterion and, in response to the determination, actuate the rejection element, wherein the rejection element is an electrostatic deflection element, an air jet, a mechanical door, or a shut off valve.

23. An apparatus comprising:
an array of electrospraying emitters including a first emitter and a second emitter; and
a controller configured to energize the first emitter for a first period of time and to energize the second emitter for a second period of time, wherein the first period of time and the second period of time are non-overlapping.

24. The apparatus of claim 23, wherein each emitter in the array of emitters has a corresponding counter-electrode.

25. The apparatus of claim 23, further comprising:
a microfluidic solution source configured to provide solution continuously to the array of emitters.

26. The apparatus of claim 23, further comprising:
a first electronic switch controlling the first emitter; and
a second electronic switch controlling the second emitter.

27. The apparatus of claim 26, wherein the controller energizes the first emitter by providing a first control signal to the first electronic switch, the first control signal pulse width modulated and having a duty cycle.

28. The apparatus of claim 27, wherein the controller is further configured to:
receive an emitter current measurement and a counter-electrode current measurement;
calculate, based on the received emitter current measurement and the received counter-electrode current measurement, a current adjustment value to compensate for parasitic current loss between the emitter and the counter-electrode; and
adjust the emitter current based on the calculated current adjustment value by modifying the duty cycle, a voltage, or a frequency of the first control signal.

29. The apparatus of claim 27, wherein the duty cycle is greater than 50 percent.

30. The apparatus of claim 29, wherein the duty cycle is about 70 or 90 percent, wherein about is within 10 percent.

31. The apparatus of claim 27, wherein the control signal includes a frequency between 1 Hertz and 10,000 Hertz.

32. The apparatus of claim 31, wherein the frequency is about 1, 100, or 1000 Hertz, wherein about is within 10 percent.

33. The apparatus of claim 23, further comprising a mixing element fluidically connected to the first emitter, the mixing element for mixing polymer and cells prior to provision to the first emitter for electrospraying.

34. The apparatus of claim 23, further comprising an image acquisition device arranged to view a region between the first emitter and a first counter electrode, the image acquisition device configured to acquire an image of the region;
wherein the controller is configured to, using the image of the region, detect a characteristic of a particle within the region.

35. The apparatus of claim 34, further comprising a rejection element operatively coupled to the controller, wherein the controller is further configured to determine that the detected characteristic does not satisfy a criterion and, in response to the determination, actuate the rejection element, wherein the rejection element is an electrostatic deflection element, an air jet, a mechanical door, or a shut off valve.

36. A method comprising:
receiving, from a first current measuring unit electrically coupled to an emitter and measuring an emitter current, an emitter current measurement;
receiving, from a second current measuring unit electrically coupled to a counter-electrode and measuring a counter-electrode current, a counter-electrode current measurement;
calculating, based on the received emitter current measurement and the received counter-electrode current measurement, a current adjustment value to compensate for parasitic current loss between the emitter and the counter-electrode; and
adjusting the emitter current based on the calculated current adjustment value.

37. The method of claim 36, wherein the first current measuring unit is a high voltage nano-ammeter.

38. The method of claim 36, wherein the emitter includes a cannula for dispersing fluid.

39. The method of claim 36, wherein the counter-electrode is arranged to receive dispersed charged solution emitted by the emitter.

40. The method of claim 36, further comprising:
spraying, by the emitter, solution into an environment having between 0.1 atmosphere and 10 atmosphere.

41. The method of claim 36, wherein calculating a current adjustment value comprises:
subtracting the measured counter-electrode current from the measured emitter current.

42. The method of claim 36, wherein adjusting the emitter current based on the calculated current adjustment value includes modifying a duty cycle of a control signal, the control signal pulse width modulated and controlling an emitter switch coupling the emitter to a power source.

43. The method of claim 42, wherein the duty cycle is greater than 50 percent.

44. The method of claim 43, wherein the duty cycle is about 70 or 90 percent, wherein about is within 10 percent.

45. The method of claim 42, wherein the control signal includes a frequency between 1 Hertz and 10,000 Hertz.

46. The method of claim 45, wherein the frequency is about 1, 100, or 1000 Hertz, wherein about is within 10 percent.

47. The method of claim 36, further comprising mixing cells and polymer in a mixing element fluidically connected to the first emitter and prior to provision to the first emitter for electrospraying.

48. The method of claim 36, further comprising an image acquisition device arranged to view a region between the first emitter and a first counter electrode, the image acquisition device configured to acquire an image of the region;
wherein the controller is configured to, using the image of the region, detect a characteristic of a particle within the region.

49. The method of claim 48, further comprising a rejection element operatively coupled to the controller, wherein the controller is further configured to determine that the detected characteristic does not satisfy a criterion and, in response to the determination, actuate the rejection element, wherein the rejection element is an electrostatic deflection element, an air jet, a mechanical door, or a shut off valve.

50. The method of claim 36, further comprising:
spraying, by the emitter, solution to form particles having a diameter between 10 nanometer and 3000 micrometers.

51. The method of claim 50, wherein the diameter is between 1 micrometer and 2500 micrometers; between 1 micrometer and 100 micrometers; between 1 micrometer and 10 micrometers; between 10 micrometers and 50 micrometers; or between 20 micrometers and 40 micrometers.

52. A method of fabricating polymer-encapsulated living cells, comprising electrospraying a population of living cells and a polymer solution using an apparatus comprising:
an electrospraying emitter;
a first current measuring unit electrically coupled to the emitter and measuring an emitter current;
a counter-electrode;
a second current measuring unit electrically coupled to the counter-electrode and measuring a counter-electrode current; and
a controller configured to:
receive an emitter current measurement and a counter-electrode current measurement;
calculate, based on the received emitter current measurement and the received counter-electrode current measurement, a current adjustment value to compensate for parasitic current loss between the emitter and the counter-electrode; and
adjust the emitter current based on the calculated current adjustment value.

53. The method of claim 52, wherein the living cells are sprayed through a first emitter, and the polymer solution is sprayed through a second emitter.

54. The method of claim 36, further comprising:
mixing a compound, therapeutic, or diagnostic with a polymer, the mixing occurring in a mixing element fluidically connected to the first emitter and prior to provision to the first emitter for electrospraying.
